(19) United States
(12) Patent Application Publication
(10) Pub. No.: US 2015/0203474 A1
(43) Pub. Date: Jul. 23, 2015

(54) SOLID FORMS COMPRISING INHIBITORS OF HCV NS5A, COMPOSITIONS THEREOF, AND USES THEREWITH

(71) Applicant: PRESIDIO PHARMACEUTICALS ,INC., San Francisco, CA (US)

(72) Inventors: Keith Lorimer, Lisburn (IE); Leping Li, Burlingame, CA (US); Min Zhong, Palo Alto, CA (US); Anna Muchnik, Belmont, CA (US)

(21) Appl. No.: 14/377,846

(22) PCT Filed: Feb. 13, 2013
(86) PCT No.: PCT/US2013/025995

§ 371 Date: Aug. 8, 2014

Related U.S. Application Data
(60) Provisional application No. 61/598,249, filed on Feb. 13, 2012.

(51) Int. Cl. C07D 403/14

(52) U.S. Cl. CPC C07D 403/14; C07B 2200/13

(57) ABSTRACT

This invention relates to: a) compounds and salts thereof that, inter alia, inhibit HCV; (b) intermediates useful for the preparation of such compounds and salts; (c) composition comprising such compounds and salts; (d) methods for preparing such intermediates, compounds, salts, and composition; (e) method of use of such compounds, salts, and compositions; and (f) kits comprising such compounds, salts, and composition.

### FIELD

Provided herein are solid forms comprising the compounds of formulae (I) and (II), compositions comprising the solid forms, methods of making the solid forms, and methods of their use in inhibiting hepatitis C virus (“HCV”) replication, including, for example, functions of the non-structural 5A (“NS5A”) protein of HCV.

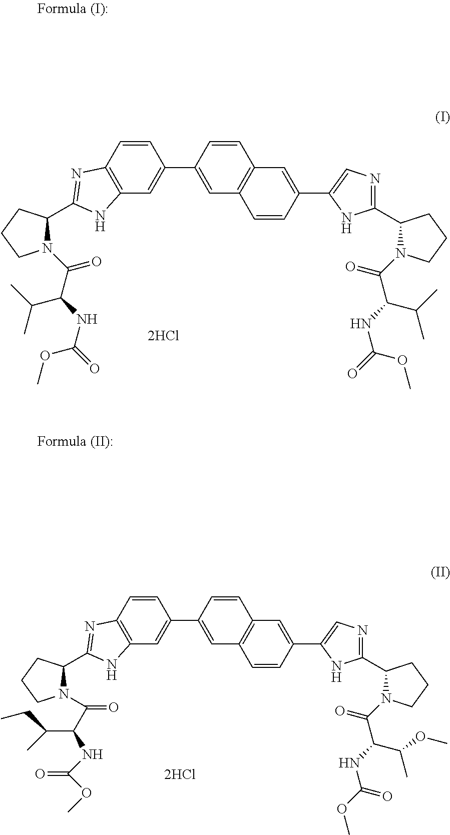 [SMILES: COC(=O)N[C@H](C(=O)N1CCC[C@H]1c1ncc(-c2ccc3cc(-c4ccc5nc([C@@H]6CCCN6C(=O)[C@@H](NC(=O)OC)C(C)C)[nH]c5c4)ccc3c2)[nH]1)C(C)C ; CC[C@H](C)[C@H](NC(=O)OC)C(=O)N1CCC[C@H]1c1nc2ccc(-c3ccc4cc(-c5cnc([C@@H]6CCCN6C(=O)[C@@H](NC(=O)OC)[C@@H](C)OC)[nH]5)ccc4c3)cc2[nH]1]

### BACKGROUND

HCV is a single-stranded RNA virus that is a member of the Flaviviridae family. The virus shows extensive genetic heterogeneity as there are currently seven identified genotypes and more than 50 identified subtypes. In HCV infected cells, viral RNA is translated into a polyprotein that is cleaved into ten individual proteins. At the amino terminus are structural proteins: the core (C) protein and the envelope glycoproteins, E1 and E2. p7, an integral membrane protein, follows E1 and E2. Additionally, there are six non-structural proteins, NS2, NS3, NS4A, NS4B, NS5A and NS5B, which play a functional role in the HCV life cycle. (see, for example, Lindenbach, B. D. and C. M. Rice, Nature, (2005) 436:933-938).

Infection by HCV is a serious health issue. It is estimated that 170 million people worldwide are chronically infected with HCV. HCV infection can lead to chronic hepatitis, cirrhosis, liver failure and hepatocellular carcinoma. Chronic HCV infection is thus a major worldwide cause of liver-related premature mortality.

The present standard of care treatment regimen for HCV infection involves interferon-alpha, alone, or in combination with ribavirin. The treatment is cumbersome and sometimes has debilitating and severe side effects and many patients do not durably respond to treatment. New and effective methods of treating HCV infection are urgently needed.

### SUMMARY

Embodiments herein provide solid forms of the compound of formulae (I) (“Compound (I)”) and (II) (“Compound (II)”).

In a first aspect, a solid form of a compound having Formula (I) is provided:

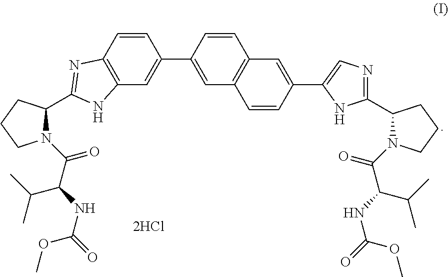 [SMILES: COC(=O)N[C@H](C(=O)N1CCC[C@H]1c1ncc(-c2ccc3cc(-c4ccc5nc([C@@H]6CCCN6C(=O)[C@@H](NC(=O)OC)C(C)C)[nH]c5c4)ccc3c2)[nH]1)C(C)C]

In a first embodiment of the first aspect, the solid form is crystalline.

In second embodiment the crystalline form is the Form A crystal form of the compound of Formula I.

In a third embodiment of the first aspect, the solid form has an XRPD pattern comprising:

a) peaks located at 1, 2, 3, 4, 5, 6, 7, 8, 9, 10, 11, 12, 13, 14, 15, 16, 17, 18, 19, 20, 21, 22, 23, 24, 25, 26, 27, 28 or all of the approximate positions identified in Table 1;

b) peaks located at 1, 2, 3, 4, 5, 6, 7, 8, 9, 10, 11, 12, 13, 14, 15, 16, 17, 18, 19, 20, 21, 22, 23, 24, 25, 26 or all of the approximate positions identified in FIG. 6;

c) peaks located 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or all of the approximate positions identified in FIG. 8; or

d) peaks located at 1, 2, 3, 4, 5, 6, 7, 8, 9, 10, 11, 12, 13, 14, 15, 16, 17, 18 or all of the approximate positions identified in FIG. 19.

In a fourth embodiment of the first aspect, the solid form has an XRPD pattern comprising peaks located at 1, 2, 3, 4 or all of the approximate positions identified in Table 2.

In a fifth embodiment of the first aspect, the solid form has an XRPD pattern comprising peaks located at values of two theta of 14.7±0.2, 17.4±0.2, and one or more of 10.6±0.2, 12.7±0.2 and 13.6±0.1 at ambient temperature, based on a high quality pattern collected with a diffractometer (CuKα) with 2θ calibrated with an NIST or other suitable standard.

In a sixth embodiment of the first aspect, pharmaceutical compositions comprising Form A is provided.

In a seventh aspect of the first aspect, a gel capsule comprising the solid form of any previous claim is provided.

In a second aspect, a solid form of a compound having Formula (II) is provided:

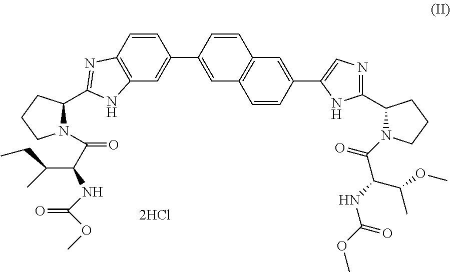 [SMILES: CC[C@H](C)[C@H](NC(=O)OC)C(=O)N1CCC[C@H]1c1nc2ccc(-c3ccc4cc(-c5cnc([C@@H]6CCCN6C(=O)[C@@H](NC(=O)OC)[C@@H](C)OC)[nH]5)ccc4c3)cc2[nH]1]

wherein the solid form is crystalline.

In first embodiment of the second aspect, the solid form is the Form I crystal form of the compound of Formula II.

In a second embodiment of the second aspect, the solid has an XRPD pattern comprising peaks located at 1, 2, 3, 4, 5, 6, 7, 8, 9, 10, 11, 12, 13, 14, 15, 16 or all of the approximate positions identified in Table 8; or peaks located at 1, 2, 3, 4, 5, 6, 7, 8, 9, 10, 11, 12 or all of the approximate positions identified in Table 9.

In a third embodiment of the second aspect, the solid has an XRPD pattern comprising peak numbers 1, 3, 13 and 17 in Table 8 and 1, 2, 3, 4, 5, 6, 7, 8, 9, 10, 11, 12 or 13 of the remaining peaks identified in Table 8.

In a fourth embodiment of the second aspect, a pharmaceutical composition comprising Form I is provided.

Without intending to be limited by any particular theory, the storage stability, compressibility, bulk density or dissolution properties of Form A of Compound I and Form I of Compound II described herein are believed to be beneficial for manufacturing, formulation and bioavailability.

The solid forms provided herein are useful as active pharmaceutical ingredients for the preparation of formulations for use in animals or humans. Thus, embodiments herein encompass the use of these solid forms as a final drug product. Certain embodiments provide solid forms useful in making final dosage forms with improved properties, e.g., powder flow properties, compaction properties, tableting properties, stability properties, and excipient compatibility properties, among others, that are needed for manufacturing, processing, formulation and/or storage of final drug products. Certain embodiments herein provide pharmaceutical compositions comprising a single-component crystal form, a multiple-component crystal form, a single-component amorphous form and/or a multiple-component amorphous form comprising the compound of formula (I) and a pharmaceutically acceptable diluent, excipient or carrier. The solid forms described herein are useful, for example, for inhibiting HCV replication, inhibiting NS5A, and treating, preventing or managing HCV infection.

### BRIEF DESCRIPTION OF THE DRAWINGS

FIG. 1 is a representative ¹H NMR spectrum of Compound I Form A.

FIG. 2 is a representative ¹³C NMR spectrum of Compound I Form A.

FIG. 3 is a representative FT-IR spectrum of Compound I Form A.

FIG. 4 is a representative DSC thermogram of Compound I Form A.

FIG. 5 is a representative X-ray powder diffraction (XRPD) pattern of Compound I Form A.

FIG. 6 is a table of the peaks represented in FIG. 5.

FIG. 7 is a representative XRPD pattern of Compound I Form A.

FIG. 8 is a table of the peaks represented in FIG. 7.

FIG. 9 is a representative XRPD pattern of Compound I Form A.

FIG. 10 is a representative XRPD pattern of Compound I Form A.

FIG. 11 is a representative ¹H NMR spectrum of Compound I Form A.

FIG. 12 is a representative XRPD pattern of Compound I Form A.

FIG. 13 is a representative ¹H NMR spectrum of Compound I Form A.

FIG. 14 is a representative DSC curve and thermogram of Compound I Form A.

FIG. 15 illustrates graphed weight % vs. relative humidity for Compound I Form A.

FIG. 16 is a representative XRPD pattern of Compound I Form A.

FIG. 17 is a representative thermogram of Compound I Form A.

FIG. 18 is a representative XRPD pattern of Compound I Form A.

FIG. 19 is a table of the peaks represented in FIG. 18.

FIG. 20 is a representative XRPD pattern of Compound I Form A.

FIG. 21 is a representative DSC curve and thermogram of Compound I Form.

FIG. 22 is a representative XRPD pattern of Compound I Form A before and after the material is stressed.

FIG. 23 is a representative DSC curve and thermogram of Compound I Form after the material is stressed.

FIG. 24 illustrates representative XRPD patterns of Compound II Form I.

FIG. 25 is a representative XRPD pattern of Compound II Form I.

FIG. 26 illustrates crystals of Compound II Form I.

FIG. 27 is a representative thermogram of Compound II Form I.

FIG. 28 is a representative DSC curve of Compound II Form I.

FIG. 29 is a DVS isotherm plot of Compound II Form I.

FIG. 30 is a DVS isotherm plot of amorphous Compound II.

FIG. 31 is a representative XRPD pattern of Compound II Form I.

FIG. 32 are polarized light microscope images of various salts of Compound I FB.

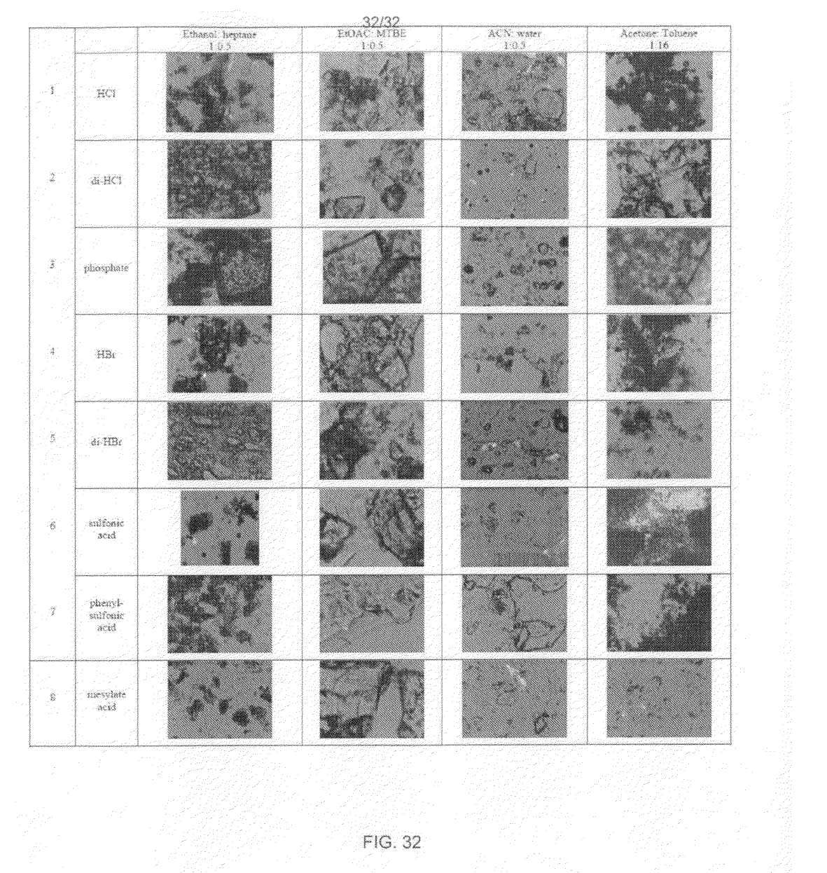

### DETAILED DESCRIPTION

### (a) Definitions

As used herein and unless otherwise specified, the terms “solid form” and related terms refer to a physical form which is not predominantly in a liquid or a gaseous state. As used herein and unless otherwise specified, the term “solid form” and related terms, when used herein to refer to Compound (I), refer to a physical form comprising Compound (I) which is not predominantly in a liquid or a gaseous state. Solid forms may be crystalline, amorphous or mixtures thereof. In particular embodiments, solid forms may be liquid crystals. A “single-component” solid form comprising Compound (I) consists essentially of Compound (I). A “multiple-component” solid form comprising Compound (I) comprises a significant quantity of one or more additional species, such as ions and/or molecules, within the solid form. For example, in particular embodiments, a crystalline multiple-component solid form comprising Compound (I) further comprises one or more species non-covalently bonded at regular positions in the crystal lattice.

As used herein and unless otherwise specified, the term “crystalline” and related terms used herein, when used to describe a substance, modification, material, component or product, unless otherwise specified, mean that the substance, modification, material, component or product is substantially crystalline as determined by X-ray diffraction. See, e.g., Remington: The Science and Practice of Pharmacy, 21^{st} edition, Lippincott, Williams and Wilkins, Baltimore, Md. (2005); The United States Pharmacopeia, 23^{rd} edition, 1843-1844 (1995).

As used herein and unless otherwise specified, the term “crystal forms” and related terms herein refer to solid forms that are crystalline. Crystal forms include single-component crystal forms and multiple-component crystal forms, and include, but are not limited to, polymorphs, solvates, hydrates, and other molecular complexes, as well as salts, solvates of salts, hydrates of salts, other molecular complexes of salts, and polymorphs thereof. In certain embodiments, a crystal form of a substance may be substantially free of amorphous forms and/or other crystal forms. In certain embodiments, a crystal form of a substance may contain less than about 1%, 2%, 3%, 4%, 5%, 6%, 7%, 8%, 9%, 10%, 15%, 20%, 25%, 30%; 35%, 40%, 45% or 50% of one or more amorphous forms and/or other crystal forms on a weight basis. In certain embodiments, a crystal form of a substance may be physically and/or chemically pure. In certain embodiments, a crystal form of a substance may be about 99%, 98%, 97%, 96%, 95%, 94%, 93%, 92%, 91% or 90% physically and/or chemically pure.

As used herein and unless otherwise specified, the terms “polymorphs,” “polymorphic forms” and related terms herein, refer to two or more crystal forms that consist essentially of the same molecule, molecules or ions. Like different crystal forms, different polymorphs may have different physical properties such as, for example, melting temperatures, heats of fusion, solubilities, dissolution rates and/or vibrational spectra, as a result of the arrangement or conformation of the molecules and/or ions in the crystal lattice. The differences in physical properties may affect pharmaceutical parameters such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to a thermodynamically more stable polymorph) or both (e.g., tablets of one polymorph are more susceptible to breakdown at high humidity). As a result of solubility/dissolution differences, in the extreme case, some solid-state transitions may result in lack of potency or, at the other extreme, toxicity. In addition, the physical properties may be important in processing (for example, one polymorph might be more likely to form solvates or might be difficult to filter and wash free of impurities, and particle shape and size distribution might be different between polymorphs).

As used herein and unless otherwise specified, the term “solvate” and “solvated,” refer to a crystal form of a substance which contains solvent. The term “hydrate” and “hydrated” refer to a solvate wherein the solvent comprises water. “Polymorphs of solvates” refers to the existence of more than one crystal form for a particular solvate composition. Similarly, “polymorphs of hydrates” refers to the existence of more than one crystal form for a particular hydrate composition. The term “desolvated solvate,” as used herein, refers to a crystal form of a substance which may be prepared by removing the solvent from a solvate.

As used herein and unless otherwise specified, the term “amorphous,” “amorphous form,” and related terms used herein, mean that the substance, component or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In other embodiments, an amorphous form of a substance may contain less than about 1%, 2%, 3%, 4%, 5%, 10%, 15%, 20%, 25%, 30%, 35%, 40%, 45% or 50% of one or more other amorphous forms and/or crystal forms on a weight basis. In certain embodiments, an amorphous form of a substance may be physically and/or chemically pure. In certain embodiments, an amorphous form of a substance may be about 99%, 98%, 97%, 96%, 95%, 94%, 93%, 92%, 91% or 90% physically and/or chemically pure.

Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder.

As used herein and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. As used herein, a tilde (i.e., “˜”) preceding a numerical value or range of values indicates “about” or “approximately.”

As used herein and unless otherwise specified, a sample comprising a particular crystal form or amorphous form that is “substantially pure,” e.g., substantially free of other solid forms and/or of other chemical compounds, or is noted to be “substantially” a crystal form or amorphous form, contains, in particular embodiments, less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or of other chemical compounds. As used herein and unless otherwise specified, a sample or composition that is “substantially free” of one or more other solid forms and/or other chemical compounds means that the composition contains, in particular embodiments, less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or other chemical compounds.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” As used herein, and unless otherwise specified, the terms “manage,” “managing” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

The preparation and selection of a solid form of a pharmaceutical compound is complex, given that a change in solid form may affect a variety of physical and chemical properties, which may provide benefits or drawbacks in processing, formulation, stability and bioavailability, among other important pharmaceutical characteristics. Potential pharmaceutical solids include crystalline solids and amorphous solids. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Whether crystalline or amorphous, potential solid forms of a pharmaceutical compound may include single-component and multiple-component solids. Single-component solids consist essentially of the pharmaceutical compound in the absence of other compounds. Variety among single-component crystalline materials may potentially arise from the phenomenon of polymorphism, wherein multiple three-dimensional arrangements exist for a particular pharmaceutical compound (see, e.g., S. R. Byrn et al., Solid State Chemistry of Drugs, (1999) SSCI, West Lafayette).

Additional diversity among the potential solid forms of a pharmaceutical compound may arise from the possibility of multiple-component solids. Crystalline solids comprising two or more ionic species are termed salts (see, e.g., Handbook of Pharmaceutical Salts: Properties Selection and Use, P. H. Stahl and C. G. Wermuth, Eds., (2002), Wiley, Weinheim). Additional types of multiple-component solids that may potentially offer other property improvements for a pharmaceutical compound or salt thereof include, e.g., hydrates, solvates, co-crystals and clathrates, among others (see, e.g., S. R. Byrn et al., Solid State Chemistry of Drugs, (1999) SSCI, West Lafayette). Moreover, multiple-component crystal forms may potentially be susceptible to polymorphism, wherein a given multiple-component composition may exist in more than one three-dimensional crystalline arrangement. The discovery of solid forms is of great importance in the development of a safe, effective, stable and marketable pharmaceutical compound.

Solid forms may exhibit distinct physical characterization data that are unique to a particular solid form, such as the crystal forms described herein. These characterization data may be obtained by various techniques known to those skilled in the art, including for example X-ray powder diffraction, differential scanning calorimetry, thermal gravimetric analysis, and nuclear magnetic resonance spectroscopy. The data provided by these techniques may be used to identify a particular solid form. One skilled in the art can determine whether a solid form is one of the forms described herein by performing one of these characterization techniques and determining whether the resulting data “matches” the reference data provided herein, which is identified as being characteristic of a particular solid form. Characterization data that “matches” those of a reference solid form is understood by those skilled in the art to correspond to the same solid form as the reference solid form. In analyzing whether data “match,” a person of ordinary skill in the art understands that particular characterization data points may vary to a reasonable extent while still describing a given solid form, due to, for example, experimental error and expected variability in routine sample-to-sample analysis. In addition to solid forms comprising Compound (I) or Compound (II), provided herein are solid forms comprising prodrugs of Compound (I) or Compound (II), also provided herein are the methods of making Compound (I) or Compound (II) and the key intermediates leading to Compound (I) or Compound (II).

A need exists for compounds having desired anti HCV therapeutic attributes, including high potency and broad genotypic coverage of most common HCV genotypes, selectivity over other targets or low toxicity and oral bioavailability. The compounds need to have safety profile suitable for chronic administration for up to a year.

To effectively use these compounds as therapeutic agents, it is desirable to have solid forms that can be readily manufactured and that have acceptable chemical and physical stability. The amorphous solid forms have as disadvantages that they absorb water and in an unpredictable fashion. Amorphous forms do not provide sufficient purity, stability or predictability in manufacturing to be useful as a pharmaceutical.

The provided solid forms (Form A of Compound I and Form I of Compound II) are sufficiently soluble in aqueous solution to allow for adequate exposure in the blood when dosed in humans. Further Form A of Compound I and Form I of Compound II were found to be sufficiently stable for reproducible manufacturing. Pharmacokinetic properties of Form A of Compound I and Form I of Compound II were found to be useful for these forms to be used as pharmaceuticals.

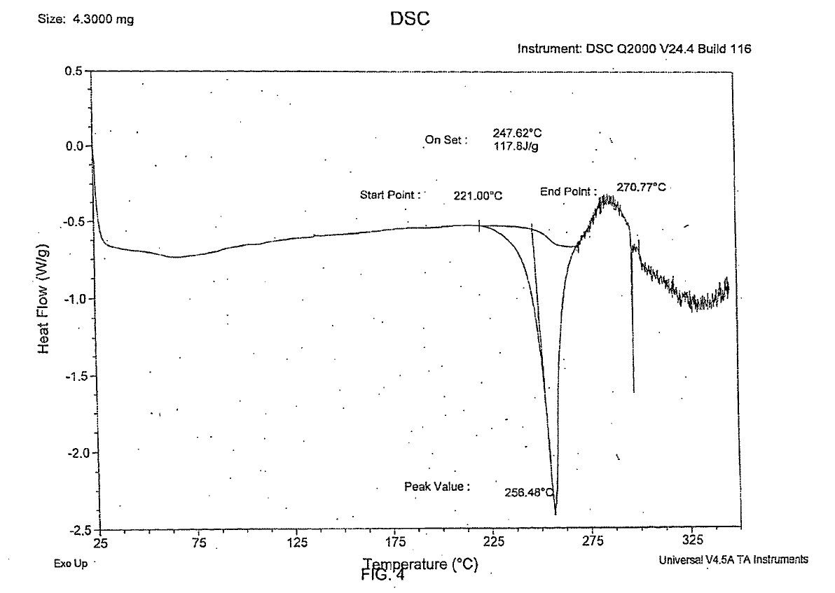

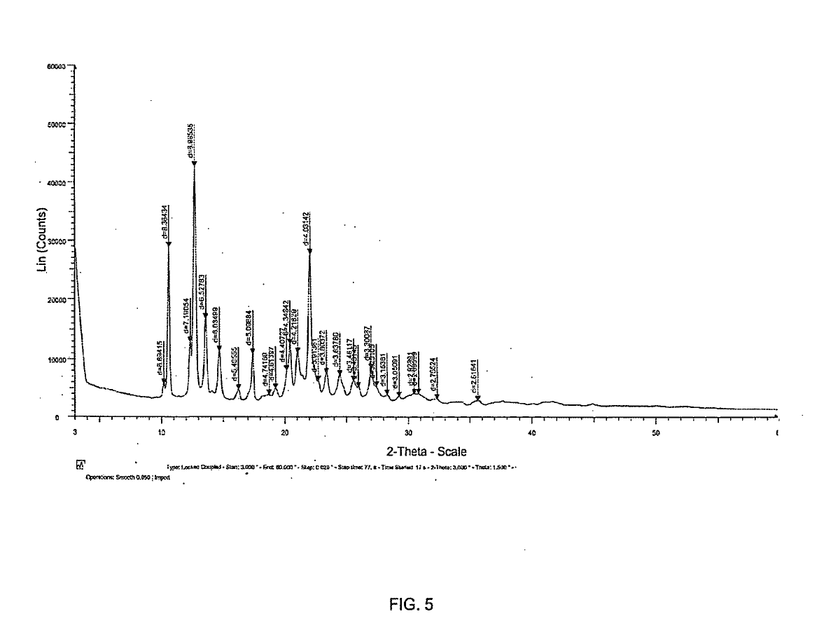

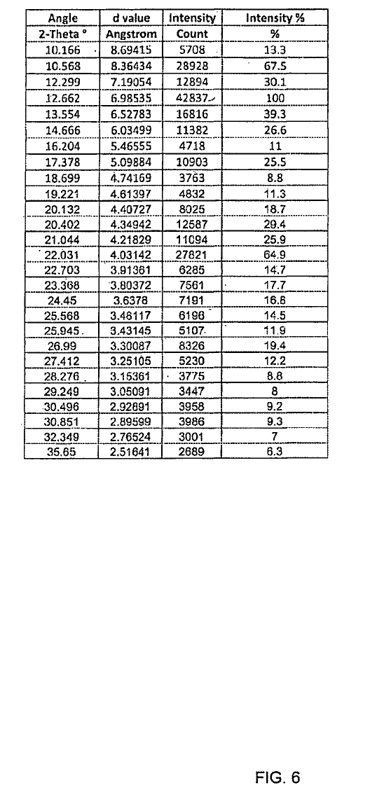

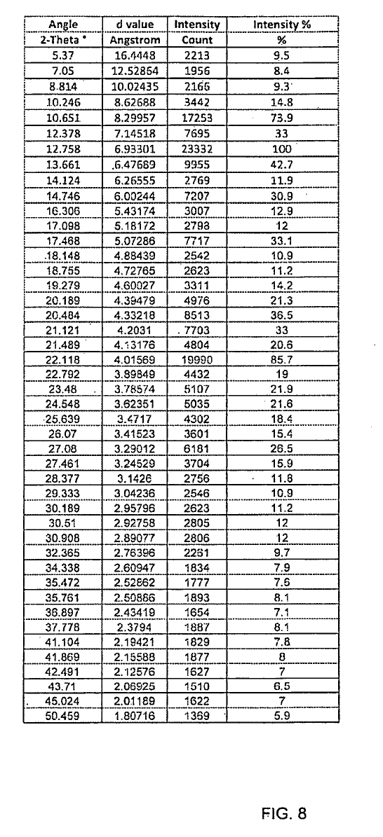

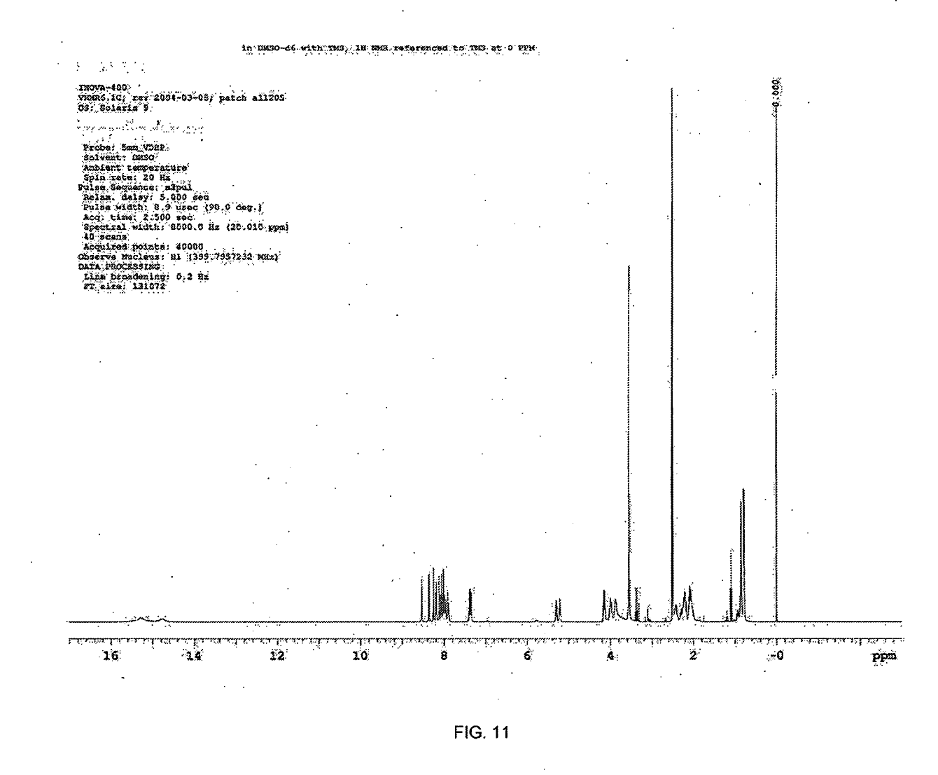

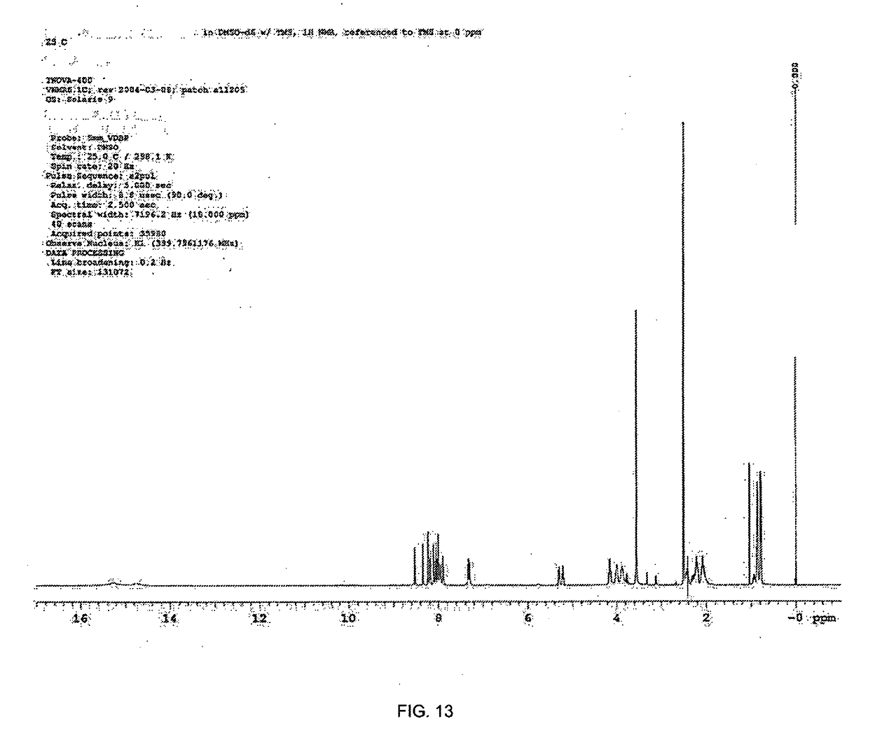

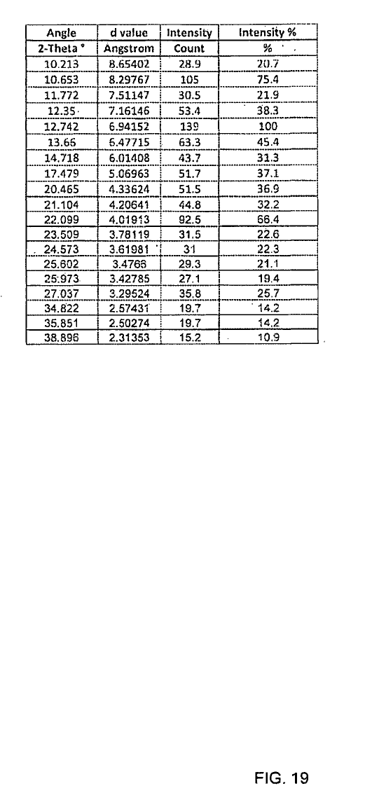

Provided herein is Form A of Compound I. Representative XRPD patterns for Form A are provided in FIGS. 5, 7, 9, 10, 12, 16 18, 20 and 22. In certain embodiments, Form A of Compound (I) is characterized by: a) peaks located at 1, 2, 3, 4, 5, 6, 7, 8, 9, 10, 11, 12, 13, 14, 15, 16, 17, 18, 19, 20, 21, 22, 23, 24, 25, 26, 27, 28 or all of the approximate positions identified in Table 1; b) peaks located at 1, 2, 3, 4, 5, 6, 7, 8, 9, 10, 11, 12, 13, 14, 15, 16, 17, 18, 19, 20, 21, 22, 23, 24, 25, 26 or all of the approximate positions identified in FIG. 6; c) peaks located 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or all of the approximate positions identified in FIG. 8; or d) peaks located at 1, 2, 3, 4, 5, 6, 7, 8, 9, 10, 11, 12, 13, 14, 15, 16, 17, 18 or all of the approximate positions identified in FIG. 19. In certain embodiments, Form A of Compound (I) is characterized by a 1, 2, 3, 4 or all of the approximate positions identified in Table 2. Representative ¹H NMR spectra for Compound Form A are provided at FIGS. 11 and 13. Representative DSC data and thermograms for Compound I Form A are provided at FIGS. 4, 14, 21 and 23.

In certain embodiments, provided herein are crystal forms of Compound (II), Form I, which are described in more detail below.

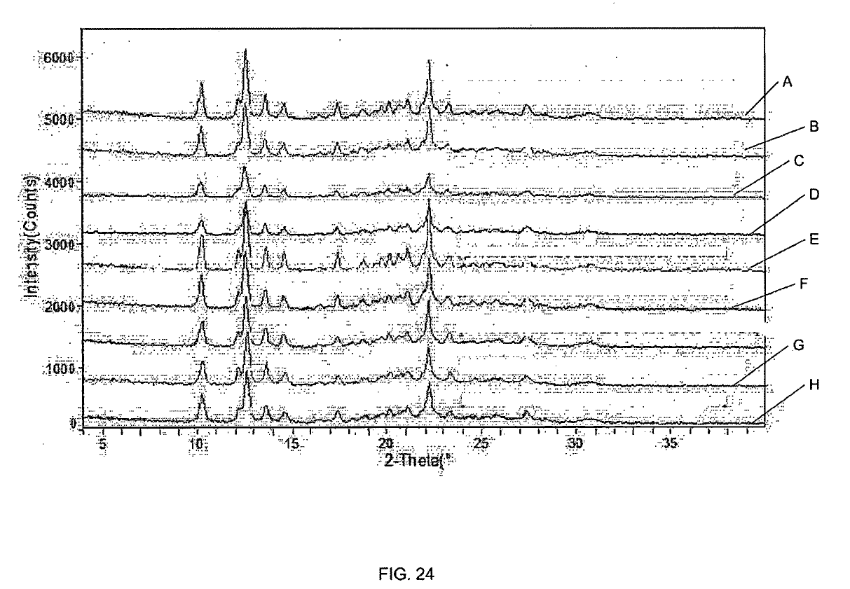

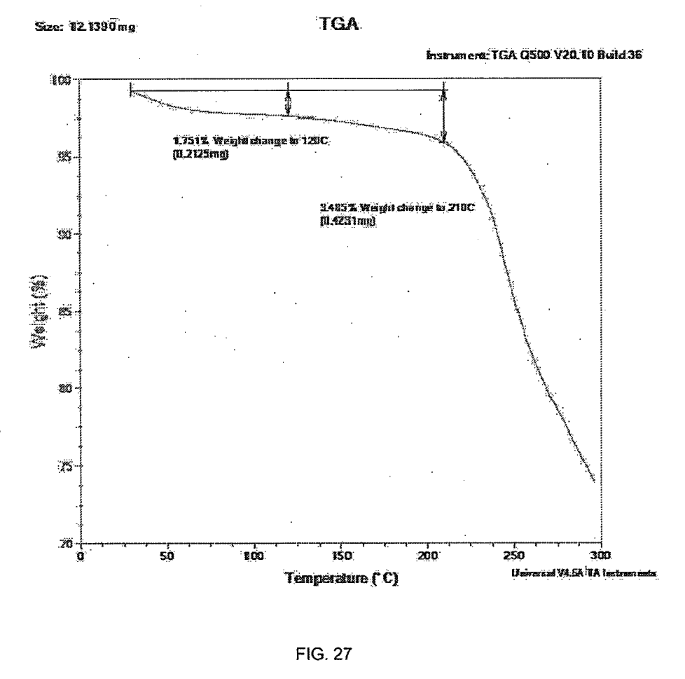

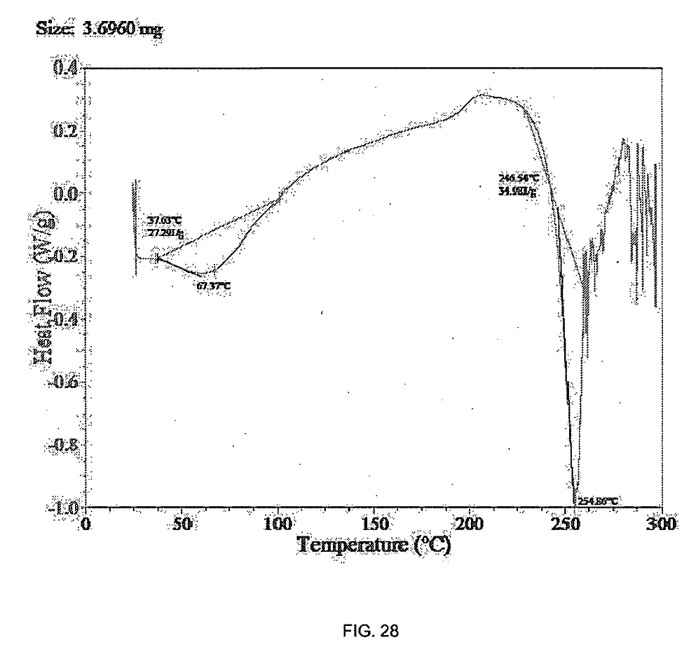

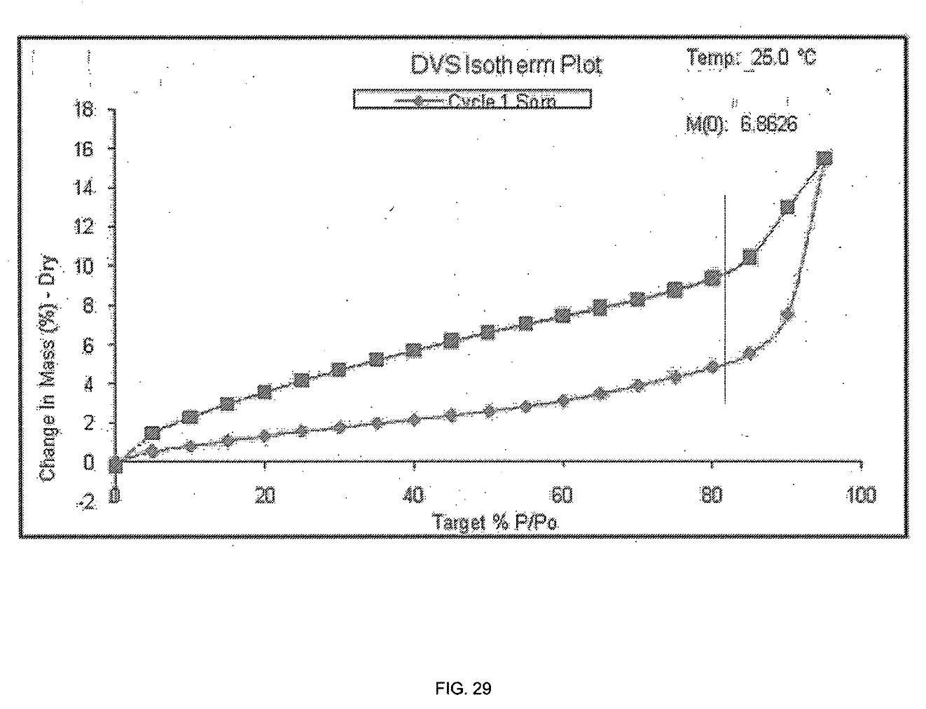

Representative XRPD patterns for Compound II Form I are provided in FIGS. 24, 25 and 31. In certain embodiments, Form I of Compound (II) is characterized by XRPD peaks located at 1, 2, 3, 4, 5, 6, 7, 8, 9, 10, 11, 12, 13, 14, 15, 16 or all of the approximate positions identified in Table 8. Representative DSC curve of Compound II Form I is provided at FIG. 28. A representative thermogram of Compound II Form I is provided at FIG. 27. A representative DVS isotherm plot of Compound II Form I is provided at FIG. 29.

Solid forms provided herein may also comprise unnatural proportions of atomic isotopes at one or more of the atoms in Compound (I) or Compound (II). For example, the compound may be radiolabeled with radioactive isotopes, such as for example deuterium (²H), tritium (³H), iodine-125 (¹²⁵I), sulfur-35 (³⁵S), or carbon-14 (¹⁴C). Radiolabeled compounds are useful as therapeutic agents, e.g., cancer therapeutic agents, research reagents, e.g., binding assay reagents, and diagnostic agents, e.g., in vivo imaging agents. All isotopic variations of Compound (I) or Compound (II), whether radioactive or not, are intended to be encompassed within the scope of the embodiments provided herein.

### (b) Synthesis and Characterization of Compounds (I) and (II)

The following abbreviations are used throughout this application:

### ACN Acetonitrile

AcOH Acetic acid

### aq Aqueous

Boc t-Butoxycarbonyl

### DCE Dichloroethane

### DCM Dichloromethane

### DIEA (DIPEA) Diisopropylethylamine

### DMA N,N-Dimethylacetamide

### DME 1,2-Dimethoxyethane

### DMF N,N-Dimethylformamide

### DMSO Dimethylsulfoxide

dppf 1,1′-Bis(diphenylphosphino)ferrocene
EDCI 1-Ethyl-3-[3-(dimethylamino) propyl]carbodiimide hydrochloride
EDTA Ethylene diamine tetraacetic acid
EC₅₀ Effective concentration to produce 50% of the maximal effect

### ESI Electrospray Ionization

Et₂O Diethyl ether

### Et₃N, TEA Triethylamine

EtOAc, EtAc Ethyl acetate

### EtOH Ethanol

### g Gram(s)

### h or hr Hour(s)

HATU 2-(7-Aza-1H-benzotriazole-1-yl)-1,1,3,3-tetramethyluronium hexafluorophosphate
HBTU O-Benzotriazol-1-yl-N,N,N′,N′-tetramethyluronium hexafluorophosphate

### Hex Hexanes

### HOBt 1-Hydroxybenzotriazole

IC₅₀ The concentration of an inhibitor that causes a 50% reduction in a measured activity

### IPA 2-Propanol

IPOAc Isopropyl acetate

### LC-MS Liquid Chromatography Mass Spectrometry

MEK Methyl ethyl ketone

### MeOH Methanol

### min Minute(s)

mmol Millimole(s)

### Moc Methoxylcarbonyl

MTBE Methyl tert-butyl ether

### N. A. Numerical Aperture

### PG Protective Group

### 1-PrOH 1-Propanol

rt Room temperature
TFA Trifluoroacetic acid

### THF Tetrahydrofuran

### TLC Thin Layer Chromatography

Solid forms of compounds I and compound II are characterized using various techniques and instruments, the operation of which and the analysis of the raw data are well known to those of ordinary skill in the art. Examples of characterization methods include, but not limited to, X-Ray Powder Diffreaction, Differential Scanning Calorimetry, Thermal Gravimetric Analysis and Hot Stage techniques.

One of ordinary skill in the art will appreciate that any of these measurements, such as the X-Ray diffraction pattern, may be obtained with a measurement error that is dependent upon the conditions that measurement is taken, the change of instrument model. The ability of ascertain substantial identity of a solid form based on data collected from multiple analytical means is within the purview of one of ordinary skill in the art.

### Instrumental Techniques

### Differential Scanning Calorimetry (DSC)

DSC analysis was performed using a TA Instruments 2920 (or other models such as Q2000) differential scanning calorimeter equipped with a refrigerated cooling system (RCS). Temperature calibration was performed using NIST traceable indium metal. The sample was placed into an aluminum DSC pan, and the weight was accurately recorded. The pan was covered with a lid, and the lid was crimped. A weighed, crimped aluminum pan was placed on the reference side of the cell. The sample cell was equilibrated at −30° C. and heated under a nitrogen purge at a rate of 2-10° C./minute, up to a final temperature of 250° C. Reported temperatures are at the transition maxima.

Modulated DSC (“MDSC”) data were obtained using a modulation amplitude of ±0.8° C. and a 60 second period with an underlying heating rate of 2° C./minute from −50 to 200° C.

For cyclic DSC analysis, the sample cell was equilibrated at ambient temperature, then cooled under nitrogen at a rate of 20° C./min to −60° C. The sample cell was held at this and then allowed to heat and equilibrate at 125° C. It was cooled again at a rate of 20° C./min to −60° C. The sample cell was held at this temperature, and it was again heated at a rate of 20° C./min to a final temperature of 250° C.

### Dynamic Vapor Sorption/Desorption (DVS)

Dynamic vapor sorption/desorption (DVS) data were collected on a VTI SGA-100 Vapor Sorption Analyzer. NaCl and PVP were used as calibration standards. Samples were not dried prior to analysis. Adsorption and desorption data were collected over a range from 5 to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Hot Stage Microscopy

Hot stage microscopy was performed using a Linkam hot stage (model FTIR 600) mounted on a Leica DM LP microscope equipped with a SPOT Insight™ color digital camera. Temperature calibrations were performed using USP melting point standards. Samples were placed on a cover glass, and a second cover glass was placed on top of the sample. As the stage was heated, each sample was visually observed using a 20×0.40 N. A. long working distance objective with crossed polarizers and a first order red compensator. Images were captured using SPOT software (v. 4.5.9).

### Thermogravimetry (TGA)

TGA analyses were performed using a TA Instruments 2950 thermogravimetric analyzer. Temperature calibration was performed using nickel and Alumel™. Each sample was placed in an aluminum pan and inserted into the TGA furnace. The furnace was heated under nitrogen at a rate of 10° C./minute to a final temperature of 350° C.

### X-Ray Powder Diffraction (XRPD)

### Inel XRG-3000 Diffractometer

XRPD patterns were collected using an Inel XRG-3000 diffractometer equipped with a curved position sensitive detector with a 2θ range of 120°. An incident beam of Cu Kα radiation (40 kV, 30 mA) was used to collect data in real time at a resolution of 0.03° 2θ. Prior to the analysis, a silicon standard (NIST SRM 640c) was analyzed to verify the Si 111 peak position. Samples were prepared for analysis by packing them into thin-walled glass capillaries. Each capillary was mounted onto a goniometer head and rotated during data acquisition. In general, the monochromator slit was set at 5 mm by 160 μM, and the samples were analyzed for 5 minutes.

### Bruker D-8 Discover Diffractometer

XRPD patterns were also collected using a Bruker D-8 Discover diffractometer and Bruker's General Detector System (GADDS, v. 4.1.20). An incident microbeam of Cu Kα radiation was produced using a fine-focus tube (40 kV, 40 mA), a Gael mirror, and a 0.5 mm double-pinhole collimator. Prior to the analysis, a silicon standard (NIST SRM 640c) was analyzed to verify the Si 111 peak position. The sample was packed between 3 μm thick films to form a portable, disc-shaped specimen. The prepared specimen was loaded in a holder secured to a translation stage. A video camera and laser were used to position the area of interest to intersect the incident beam in transmission geometry. The incident beam was scanned and rastered to optimize orientation statistics. A beam-stop was used to minimize air scatter from the incident beam. Diffraction patterns were collected using a Hi-Star area detector located 15 cm from the sample and processed using GADDS. The intensity in the GADDS image of the diffraction pattern was integrated using a step size of 0.04° 2θ. The integrated patterns display diffraction intensity as a function of 2θ.

### PANalytical EXPERT Pro MPD Diffractometer

The XRPD patterns were collected using a PANalytical X'Pert Pro diffractometer. An incident beam of Cu Kα radiation was produced using an Optix long, fine-focus source. An elliptically graded multilayer mirror was used to focus the Cu Kα X-rays of the source through the specimen and onto the detector. Data were collected and analyzed using X'Pert Pro Data Collector software (v. 2.2b). Prior to the analysis, a silicon specimen (NIST SRM 640c) was analyzed to verify the Si 111 peak position. The specimen was sandwiched between 3 μm thick films, analyzed in transmission geometry, and rotated to optimize orientation statistics. A beam-stop, short anti scatter extension, and anti scatter knife edge were used to minimize the background generated by air scattering. Soller slits for the incident and diffracted beams were used for the incident and diffracted beams to minimize axial divergence. Diffraction patterns were collected using a scanning position-sensitive detector (X'Celerator) located 240 mm from the specimen and Data Collector software v. 2.2b.

### Shimadzu XRPD-6000 Diffractometer

XRPD patterns were collected using a Shimadzu XRPD-6000 X-ray powder diffractometer. An incident beam of Cu Kα radiation was produced using a long, fine-focus X-ray tube (40 kV, 40 mA) and a curved graphite monochromator. The divergence and scattering slits were set at 1°, and the receiving slit was set at 0.15 mm. Diffracted radiation was detected by a NaI scintillation detector. Data were collected and analyzed using XRPD-6100/7000 software (v. 5.0). Prior to the analysis, a silicon standard (NIST SRM 640c) was analyzed to verify the Si 111 peak position. Samples were prepared for analysis by placing them in an aluminum holder with a silicon zero-background insert. Patterns were typically collected using a θ-2θ continuous scan at 3°/min. (0.4 sec/0.02° step) from 2.5 to 40° 2θ.

### Proton Nuclear Magnetic Resonance (NMR)

The solution ¹H NMR spectrum was primarily acquired at ambient temperature with a Varian^{UNITY}INOVA-400 spectrometer at a ¹H Larmor frequency of approximately 400 MHz. The sample was typically dissolved in d⁶-DMSO or CD₃OD containing tetramethylsilane (TMS) as reference.

### Example

### Comparison of Compound I with Other Salt Forms of Compound I Free Base (“Compound I FB”)

Several salts of the Compound I FB:

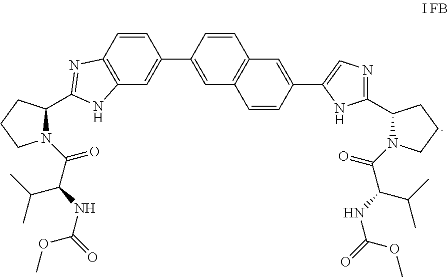 [SMILES: COC(=O)N[C@H](C(=O)N1CCC[C@H]1c1ncc(-c2ccc3cc(-c4ccc5nc([C@@H]6CCCN6C(=O)[C@@H](NC(=O)OC)C(C)C)[nH]c5c4)ccc3c2)[nH]1)C(C)C]

were made in order to arrive at Compound I. Based on solubility screening of Compound I FB, four mixed solvents were selected as the solvents to prepare stock solutions and used for salt screening: ethanol/heptanes (1/0.5 (v/v)), EtOAc/MTBE (1/0.5 (v/v)), ACN/water (1/0.5 (v/v)) and acetone/toluene (1/16 (v/v)). Approximately 25 mg of Compound I FB was weighed into each of 32 vials, and then each of the mixed solvents was used to dissolve the samples in 8 of the vials. Counter ion in equivalent molar ratios of the test counter ions (HCl, di-HCl, phosphate, HBr, di-HBr, sulfonic acid, phenylsulfonic acid and mesylate acid) were added. The ratio was set to two to one for di-HCl and di-HBr. The physical observations of each sample are shown below in Table 16:

TABLE 16
Physical observation of different salts after counterions were added

| | | Ethanol/ | EtOAC/ | | |
|---|---|---|---|---|---|
| | | heptane | MTBE | ACN/water | Acetone/Toluene |
| Sample | Counter ion | 1/0.5 (v/v) | 1/0.5 (v/v) | 1/0.5 (v/v) | 1/16 (v/v) |
| 1 | HCl | Clear | Turbid | Clear | Turbid |
| 2 | 2HCl | Clear | Turbid | Clear | Turbid |
| 3 | Phosphate | Clear | Turbid | Clear | Turbid |
| 4 | HBr | Clear | Turbid | Clear | Turbid |
| 5 | 2HBr | Clear | Delamination + | Clear | Turbid + many |
| | | | oil + turbid | | particles |
| 6 | sulfonic acid | Turbid + | Turbid + | Clear | Turbid + many |
| | | many particles | many particles | | particles |
| 7 | phenylsulfonic acid | Delamination | Delamination + | Clear | Turbid + many |
| | | | oil + turbid | | particles |
| 8 | Mesylate acid | Delamination | Turbid + oil | Clear | Turbid + many |
| | | | | | particles |

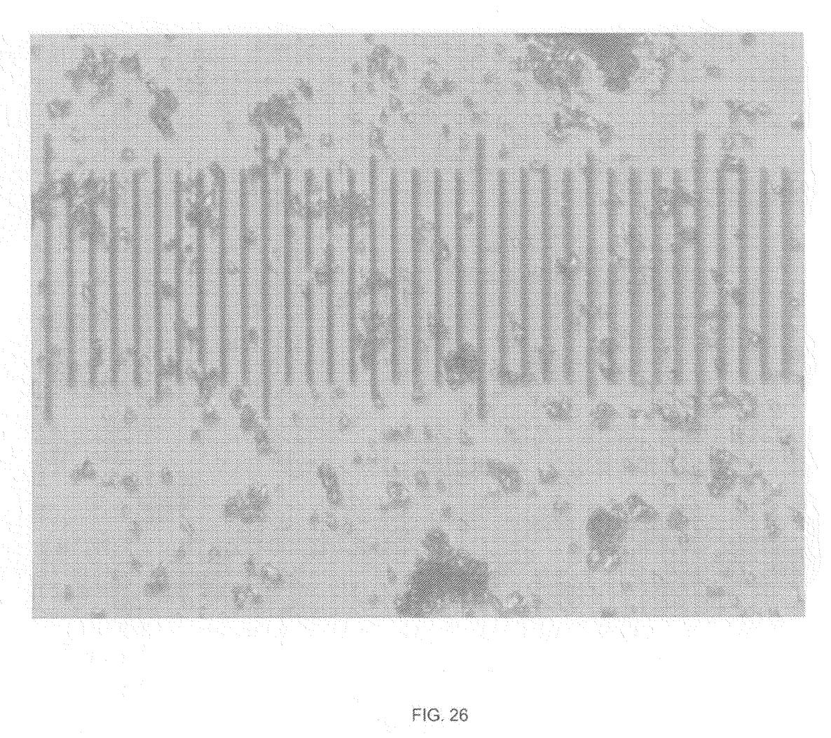

Ethanol/heptane=1/0.5 (v/v) and EtOAc/MTBE=1/0.5 (v/v) could produce solids for sulfate. Acetone/toluene=1/16 (v/v) could produce solids for di-HBr salt, sulfate, phenylsulfonic salt and mesylate. The resulting solids after slow evaporation were further characterized by microscopic observation. Microscopy was performed using a Leica DMLP polarized light microscope equipped with 2.5×, 10× and 20× objectives and a digital camera to capture images showing particle shape, size, and crystallinity. Crossed polars were used to show birefringence and crystal habit for the samples dispersed in immersion oil. As can be seen in FIG. 26 only non-birefrigent solids could be observed. The di-HCl salt of Compound I FB (thus Compound I) was selected for further evaluations on crsytaline formation or polymorph screening.

### Example

### Synthesis of Compound I (Aka Di-HCl Salt of Compound 3-3)

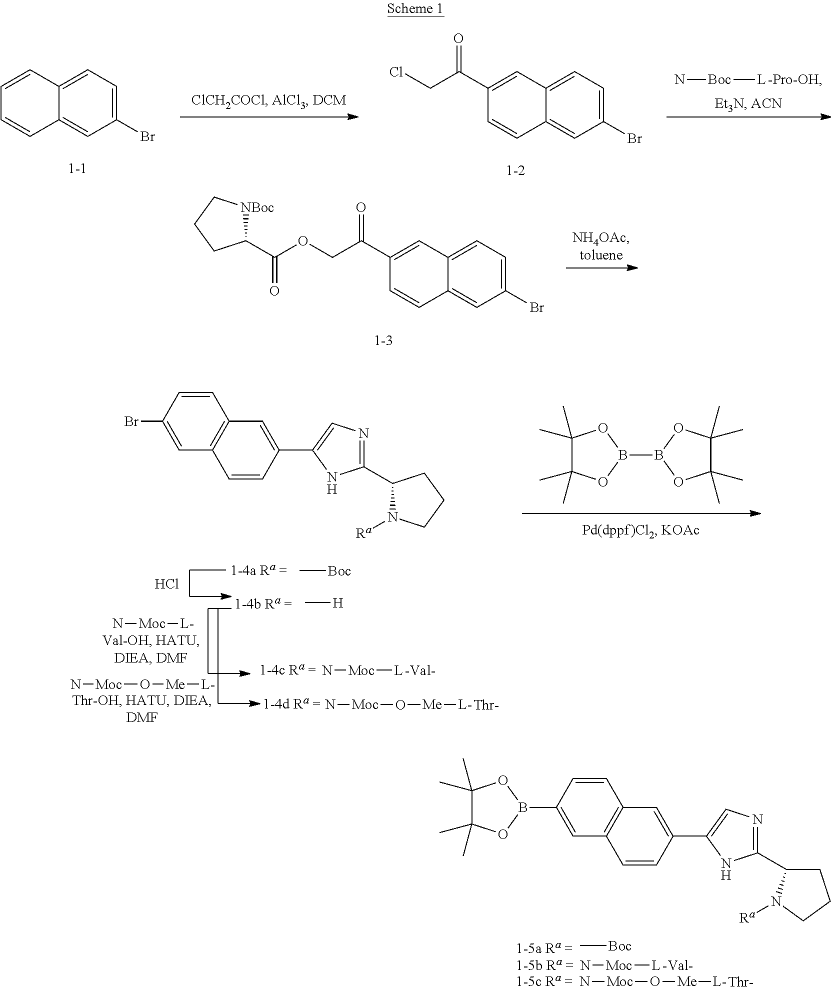

Step 1. Referring to Scheme 1. A 100 L QVF reactor under nitrogen atmosphere was charged with DCM (35.0 L, 10.0 volume). After the reaction mass was cooled to 10-15° C., anhydrous AlCl₃ (2.65 kg, 1.1 eq.) was added portion wise over a period of 90-120 min. Subsequently, the reaction mixture was cooled to 0° C. and ClCH₂COCl (1.51 L, 1.05 eq.) was slowly added over a period of 90-120 min with stirring for complete dissolution. Separately, DCM (35.0 L, 10.0 volume) and 2-bromonaphthalene (3.50 kg, 1.0 eq.) were charged into a 200 L Glass Lined Reactor (GLR) under nitrogen atmosphere and the resulting mass was cooled to 0-5° C. Next, the first prepared solution in a 100 L QVF was added slowly through a dropping funnel to the 200 L GLR over a period of 2-3 hrs while maintaining the internal temperature between 0-5° C. The reaction mass was stirred at this temperature for >60 min and monitored by HPLC analysis. After >95% of 2-bromonaphthalene was consumed as determined by HPLC analysis, cold water (70.0 L, 2.0 volume) was carefully added into the 200 L GLR reactor with stirring to quench the reaction. The CH₂Cl₂ layer was separated, washed thrice with purified water (50 L×3, 14.0 volume) and once with saturated brine (50 L×1, 14.0 volume), and dried over anhydrous Na₂SO₄. The solvent was removed under a reduced pressure (600 mmHg) and the residue was dissolved in EtOAc (17.5 L) at 60-65° C. To the clear solution was then added hexanes (35.0 L, 10.0 volume) at 65-70° C. The mixture was stirred for 1 hr and cooled to 25-30° C. gradually. The resulting mixture was filtered; the solid was washed with hexanes (1.75 L×2) and dried in a vacuum tray drier at 40-45° C. for 12 hrs to give compound 1-2 (1.88 kg, 40% yield) as off-white solid with a purity of >95% determined by HPLC. LC-MS (ESI): m/z 283.9 [M+H]⁺. ¹H NMR (500 MHz, CDCl₃): δ 8.44 (s, 1H), 8.07 (s, 1H), 8.04 (d, J=11.0 Hz, 1H), 7.84 (d, J=8.5 Hz, 2H), 7.66 (d, J=8.5 Hz, 1H), 4.81 (s, 2H) ppm.

Step 2. Compound 1-2 (3.7 kg, 1.0 eq.) and CH₃CN (74.0 L, 20.0 volume) were charged into a 200 L Stainless Steel Reactor (SSR) under nitrogen atmosphere. To the solution was slowly added Et₃N (9.10 L, 5.0 eq.) at 25-30° C. over a period of 30-45 min, followed by adding N-Boc-L-Proline (3.23 kg, 1.15 eq.) portion wise over a period of 90 min. The resulting reaction mass was stirred at 25-30° C. and monitored by HPLC. After stirring for 12 hrs, HPLC analysis indicated that >97% of compound 1-2 was consumed. Next, the reaction mass was concentrated at 40-45° C. under vacuum (600 mmHg) to remove CH₃CN; the resulting syrup was added with purified water (50.0 L) and extracted twice with EtOAc (25 L×2). The organic extracts were washed twice with purified water (25 L×2) and once with saturated brine (25.0 L). Subsequently, the organic layer was dried over anhydrous Na₂SO₄ and concentrated initially under house vacuum (600 mmHg) and finally under high vacuum to give compound 1-3 (5.50 kg, 91% yield) as brown colored semi solid with a purity of >92.0% determined by HPLC analysis. LC-MS (ESI): m/z 463.1 [M+H]⁺. ¹H NMR (400 MHz, d⁶-DMSO): δ 8.74 (s, 1H), 8.30 (s, 1H), 7.91-8.07 (m, 3H), 7.75 (d, J=8.4 Hz, 1H), 5.54-5.73 (m, 2H), 4.34 (m, 1H), 3.30-3.37 (m, 3H), 2.23-2.29 (m, 1H), 2.12-2.15 (m, 1H), 1.81-1.95 (m, 2H), 1.30 (m, 9H) ppm.

Step 3. Compound 1-3 (5.50 kg, 1.0 eq.) and toluene (55 L, 10.0 volume) were charged into a 200 L SSR under an atmosphere of nitrogen. To the resulting reaction mass was added NH₄OAc (9.20 kg, 10.0 eq.) at 25-30° C. under an atmosphere of nitrogen. Next, the reaction mass was heated at 110-115° C. and water generated in the reaction was azeotropically removed. After >97% of compound 1-3 was consumed as determined by HPLC analysis, the reaction mass was concentrated under vacuum (600 mmHg) to completely remove toluene and was cooled to ˜25-30° C. The residue was diluted with EtOAc (55.0 L, 10.0 volume) and purified water (55.0 L, 10.0 volume) with stirring. The organic layer was separated, washed twice with purified water (25 L×2) and once with saturated brine (25 L×1), and dried over anhydrous Na₂SO₄. On removal of the drying agent, the solvent was removed under vacuum (600 mmHg) at 40-45° C. to give a crude product, which was stirred with MTBE (2.0 volume) for 1 hr and filtered. The solid was washed with cold MTBE (2.75 L, 0.5 volume) and dried in a vacuum tray drier at 40-45° C. for 12 hrs to give compound 1-4a (3.85 kg, 73% yield) as pale yellow solid with a purity of >99.0% determined by HPLC analysis and an enantiomeric purity of >99.7% determined by chiral HPLC analysis (Chiralpak AD-H (250×4.6 mm), Eluent: hexanes/EtOH=80/20 (v/v), Flow rate: 0.7 mL/min). LC-MS (ESI): m/z 443.1 [M+H]⁺. ¹H NMR (400 MHz, d⁶-DMSO): δ 8.23 (s, 1H), 8.10 (s, 1H), 7.93 (m, 1H), 7.84 (m, 2H), 7.54-7.56 (m, 2H), 4.77-4.85 (, 1H), 3.53 (m, 1H), 3.36 (m, 1H), 2.16-2.24 (m, 1H), 1.84-1.99 (m, 3H), 1.39 and 1.10 (s, s, 9H) ppm.

Step 4. Compound 1-4a (3.85 kg, 1.0 eq.) and 1,4-dioxane (58.0 L, 15.0 volume) were charged into a 200 L SSR under an atmosphere of nitrogen. Next, bis(pinacalato)diboron (2.43 kg, 1.1 eq.), KOAc (2.56 kg, 3.0 eq.) and Pd(dppf)Cl₂ (285.0 g, 0.04 eq.) were charged into the SSR at 25-30° C. under an atmosphere of nitrogen. The resulting reaction mass was degassed with nitrogen at 25-30° C. for 30-45 min. Subsequently, the reaction mass was stirred at 75-80° C. for 4-5 hrs and monitored by HPLC analysis. After >97% of compound 1-4a was consumed, the reaction mass was concentrated to remove dioxane initially under vacuum (600 mmHg) and finally under high vacuum at 45-50° C. Water (35.0 L) and EtOAc were added with stirring. Layers were separated, and the organic layer was washed with saturated brine solution (25.0 L), treated with active charcoal and filtered through a Celite™545 pad. The filtrate was concentrated; the residue was then purified by precipitation from MTBE (5.0 L, 10.0 volume) to give compound 1-5a (3.10 kg, 73% yield) as pale yellow solid with a purity of >96.0% determined by HPLC analysis. LC-MS (ESI): m/z 490.3 [M+H]⁺.

Synthesis of compound 1-4b. To a solution of compound 1-4a (2.0 g, 4.5 mmol) in dioxane (25 mL) was added 4.0 N HCl in dioxane (25 mL). After stirring at rt for 4 hrs, the reaction mixture was concentrated and the residue was dried in vacuo to give compound 1-4b (2.1 g) as yellow solid, which was used without further purification. LC-MS (ESI): m/z 342.1 [M+H]⁺.

Synthesis of compound 1-4c. A mixture of compound 1-4b (2HCl salt; 1.87 g, 4.5 mmol) in DMF (25 mL) was added HATU (2.1 g, 5.4 mmol), DIPEA (3.7 mL, 22.5 mmol) and N-Moc-L-Valine (945 mg, 5.4 mmol). After stirring at rt for 15 min, the reaction mixture was slowly added to cold water (400 mL). The resulting suspension was filtered; the solid was washed with cold water and dried in vacuo to give compound 1-4c (2.2 g, 98% yield) as white solid. LC-MS (ESI): m/z 500.1 [M+H]⁺.

Synthesis of compound 1-4d. Following the procedure as described for the synthesis of compound 1-4c and replacing N-Moc-L-Valine with N-Moc-O-Me-L-Threonine, compound 1-4d was obtained. LC-MS (ESI): m/z 516.1 [M+H]⁺.

Synthesis of compound 1-5b. Following the procedure as described for the synthesis of compound 1-5a and replacing compound 1-4a with 1-4c, compound 1-5b was obtained. LC-MS (ESI): m/z 547.3 [M+H]⁺.

Synthesis of compound 1-5c. Following the procedure as described for the synthesis of compound 1-5a and replacing compound 1-4a with 1-4d, compound 1-5c was obtained. LC-MS (ESI): m/z 563.3 [M+H]⁺.

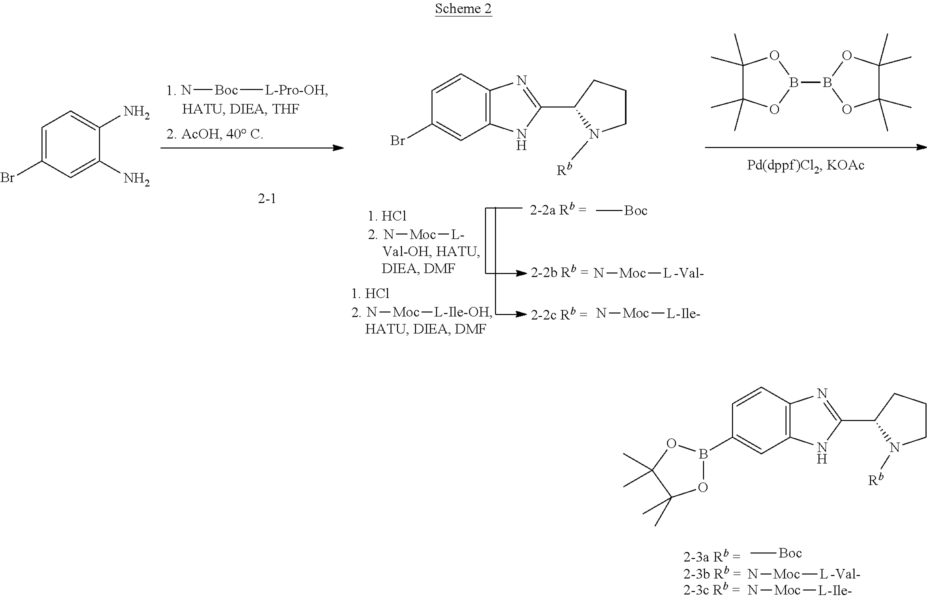

Step 1. Referring to Scheme 2, N-Boc-L-Proline (4.02 kg, 1.0 eq.) and THF (52.5 L, 15.0 volume) were charged into a 200 L reactor under nitrogen atmosphere. The mixture was cooled to 20-25° C. and N, N-diisopropylethylamine (4.8 L, 1.5 eq.) was added over a period of 60 min. Next, HATU (7.11 kg, 1.0 eq.) was slowly added by portion wise over a period of 90-120 min at 20-25° C. under an atmosphere of nitrogen. After stirring at the same temperature for 15 min, 4-bromo-1,2-diaminobenzene (3.50 kg, 1.0 eq.) was added into the reactor portion-wise over a period of 90-120 min. The resulting reaction mass was stirred at the same temperature. After stirring for 4-5 hrs, HPLC analysis indicated that >97% of 4-bromo-1,2-diaminobenzene was consumed. The reaction mass was concentrated under vacuum (600 mmHg) to remove THF at <40° C. and the residue was diluted with ethyl acetate (40.0 L, 10.0 volume) and purified water (25.0 L, 7.0 volume). The resulting mixture was well stirred and the organic layer was separated. Subsequently, the organic layer was washed with purified water (25 L×3, 7.0 volume) and with saturated brine solution (25 L×1, 7.0 volume) and dried over anhydrous Na₂SO₄. The solvent was removed under high vacuum at <40° C. to give an intermediate, which was dissolved in glacial AcOH (24.5 L, 7.0 volume). The resulting mixture was stirred at 40-42° C. and monitored by HPLC. After stirring for 10-12 hrs, HPLC analysis indicated >0.97% of the intermediate was consumed. AcOH was completely distilled off under high vacuum at 40-45° C. The resulting syrup mass was diluted with EtOAc (50.0 L, 14.0 volume) and was purified by washing with water (25.0 L, 7.0 volume) with stirring. The organic layer was separated, washed twice with 5.0% (w/w) aqueous NaHCO₃ solution (25.0 L×2, 7.0 volume), twice with purified water (25.0 L×2) and once with saturated brine (25 L×1, 7.0 volume), and dried over anhydrous Na₂SO₄. The solution was treated with active carbon before it was filtered and concentrated under vacuum (600 mmHg) at 40-45° C. to give crude product as a foamy solid (5.20 kg). The residue was suspended with stirring in MTBE (5.2 L, 1.5 volume), the solid was collected by filtration, washed with MTBE (1.75 L, 0.5 volume) and dried in a vacuum tray drier at 40-45° C. for 12 hrs to give compound 2-2a (4.20 kg, 63% yield) as pale brown solid with a purity of >98.0% determined by HPLC analysis. LC-MS (ESI): m/z 366.1 [M+H]⁺. ¹H NMR (400 MHz, d⁶-DMSO): δ 12.40 (m, 1H), 7.58-7.70 (m, 1H), 7.37-7.46 (m, 1H), 7.24 (m, 1H), 4.85-4.94 (m, 1H), 3.54 (, 1H), 3.35-3.53 (m, 1H), 2.20-2.32 (m, 1H), 1.88-1.96 (m, 3H), 1.38 and 0.98 (s, s, 9H) ppm.

Step 2. To a mixture of compound 2-2a (5.05 g, 13.8 mmol), bis(pinacolato)diboron (7.1 g, 27.9 mmol), and KOAc (3.2 g, 32.5 mmol) in 1,4-dioxane (100 mL) was added Pd(dppf)Cl₂ (400 mg, 0.5 mmol) under an atmosphere of nitrogen. After stirring at 80° C. for 3 hrs under an atmosphere of nitrogen, the reaction mixture was concentrated. The residue was purified by silica gel column chromatography (Petroleum ether/EtOAc=2/1(v/v)) to give compound 2-3a (3.0 g, 53% yield) as gray solid. LC-MS (ESI): m/z 414.2 [M+H]⁺.

Synthesis of compound 2-2b. To a solution of compound 2-2a (4.0 g, 10.9 mmol) in dioxane (40 mL) was added 4 N HCl in dioxane (40 mL). After stirring at rt overnight, the reaction mixture was concentrated. The residue was washed with DCM, filtered, and dried in vacuo to afford a hydrochloride salt in quantitative yield. Subsequently, the salt (10.9 mmol) was dissolved in DMF (30 mL), the resulting solution was added DIPEA (5.8 mL, 33.0 mmol), followed by adding N-Moc-L-Valine (2.1 g, 12.1 mmol) and HATU (4.6 g, 12.1 mmol). After stirring at rt for 1 hr, the reaction mixture was partitioned between H₂O and DCM. The organic phase was consequently washed with H₂O and brine, dried over anhydrous Na₂SO₄, filtered, and concentrated. The residue was purified by silica gel column chromatography (DCM/Petroleum ether=4/1 (v/v)) to give compound 2-2b (3.0 g, 65% yield). LC-MS (ESI): m/z 424.1 [M+H]⁺.

Synthesis of compound 2-c. Following the same procedure as that for preparing compound 2-2b and replacing N-Moc-L-Valine with N-Moc-L-Isoleucine, compound 2-2c was obtained. LC-MS (ESI): m/z 438.1 [M+H]⁺.

Synthesis of compound 2-3b. Following the procedure as described for the synthesis of compound 2-3a and replacing compound 2-2a with 2-2b, compound 2-3b was obtained. LC-MS (ESI): m/z 471.3 [M+H]⁺.

Synthesis of compound 2-3c. Following the procedure as described for the synthesis of compound 2-3a and replacing compound 2-2a with 2-2c, compound 2-3c was obtained. LC-MS (ESI): m/z 485.3 [M+H]⁺.

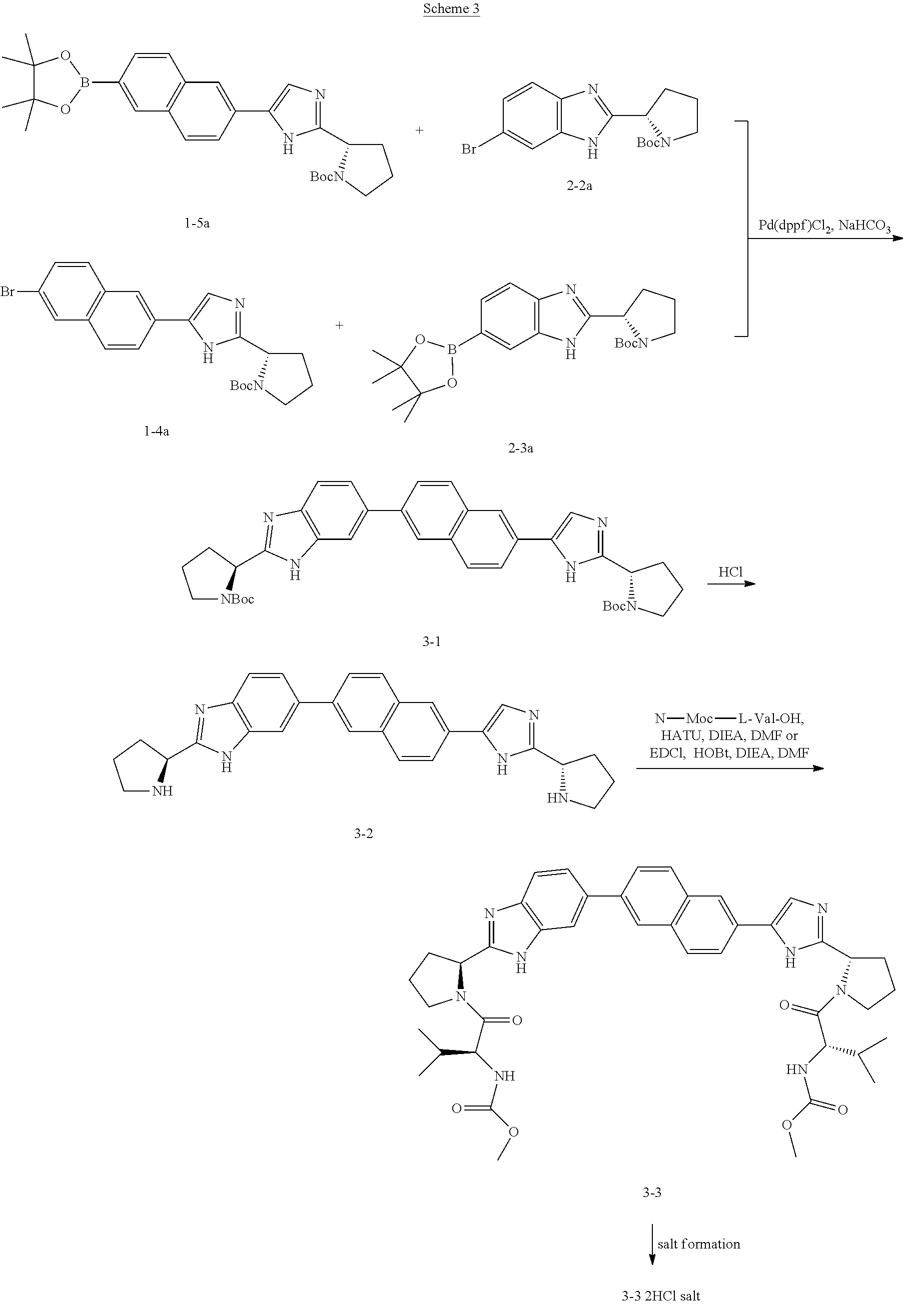

Step 1. Referring to Scheme 3, compounds 1-5a (1.3 kg, 1.0 eq.), 2-2a (975.0 g, 1.0 eq.), NaHCO₃ (860.0 g, 3.80 eq.), Pd(dppf)Cl₂ (121.7 g, 0.05 eq.), purified water (5.2 L, 4.0 volume) and 1,2-dimethoxy ethane (DME) (24.7 L, 19.0 volume) were charged into a 50.0 L 4-necked round bottom flask under argon atmosphere. After being degassed using argon for a period of 30 min, the reaction mass was slowly heated to ˜80° C. and stirred at this temperature for 12-14 hrs. HPLC analysis indicated that >97% of compound 2-2a was consumed. Next, the reaction mass was concentrated to completely remove DME under vacuum (600 mmHg) at 40-45° C. and the residue was diluted with 20% (v/v) MeOH in DCM (13.0 L, 10 volume) and purified water (13.0 L, 10.0 volume) with stirring. The organic layer was separated and the aqueous layer was extracted with 20% (v/v) MeOH in DCM (6.5 L×2, 10.0 volume). The combined organic extracts were washed twice with water (6.5 L×2, 10.0 volume) and once with saturated brine (6.5 L, 5.0 volume) and dried over anhydrous Na₂SO₄. The solvent was removed under vacuum (600 mmHg) and the residue was purified by flash column chromatography using silica gel with hexanes/EtOAc as eluent to give compound 3-1 (1.0 kg, 63% yield) as off white solid with a purity of >98.0% determined by HPLC analysis. LC-MS (ESI): m/z 649.3 [M+H]⁺. ¹H NMR (400 MHz, d⁶-DMSO): δ 12.26-12.36 (m, 1H), 11.88-11.95 (m, 1H), 8.23 (s, 1H), 8.11 (s, 1H), 7.91 (m, 3H), 7.85-7.87 (m, 2H), 7.51-7.81 (m, 3H), 4.78-4.99 (m, 2H), 3.55-3.59 (m, 2H), 3.35-3.44 (m, 2H), 2.30-2.47 (m, 2H), 1.85-2.01 (m, 6H), 1.39, 1.14, 1.04 (s, s, s, 18H) ppm. Alternatively, compound 3-1 can be obtained following the same procedure and using compounds 1-4a and 2-3a instead of compounds 1-5a and 2-2a as the Suzuki coupling components.

Step 2. Compound 3-1 (1.0 kg, 1.0 eq.) and IPA (7.0 L, 7.0 volume) were charged into a 20.0 L four-necked RB flask under nitrogen atm. The reaction mass was cooled to 18-20° C. and 3.0 N HCl in isopropyl alcohol (7.0 L, 7.0 volume) was added over a period of 90-120 min under nitrogen atmosphere. After stirring at 25-30° C. for 10-12 hrs under nitrogen atmosphere, HPLC analysis indicated that >98% compound 3-1 was consumed. Next, the reaction mass was concentrated to remove IPA under vacuum at 40-45° C. The semi solid obtained was added to acetone (2.0 L, 2.0 volume) with stirring and the resulting suspension was filtered under nitrogen atmosphere. The solid was washed with acetone (2.0 L, 2.0 volume) and dried in a vacuum tray drier at 40-45° C. for 10 hrs to give compound 3-2 (860 g, 94% yield) as pale yellow solid with a purity of >98.0% determined by HPLC analysis. LC-MS (ESI): m/z 449.2 [M+H]⁺. ¹H NMR (400 MHz, d⁶-DMSO): δ 10.49-10.59 (m, 2H), 10.10 and 9.75 (m, m, 2H), 8.60 (s, 1H), 8.31 (s, 2H), 8.15 (m, 1H), 8.13-8.15 (m, 2H), 7.96-8.09 (m, 2H), 7.82 (s, 2H), 5.08 (m, 2H), 3.39-3.53 (m, 4H), 2.47-2.54 (m, 3H), 2.37 (m, 1H), 2.14-2.21 (m, 2H), 2.08 (m, 2H) ppm.

Step 3. Compound 3-2 (2.2 kg, 1.0 eq.) was added to a four necked round bottom flask charged with DMF (4.4 L, 20.0 volume) under a nitrogen atmosphere. After stirring for 15 min, the mixture was added N-Moc-L-Valine (226.2 g, 3.52 eq.) in one lot at 25-30° C. Next, the mixture was cooled to −20 to −15° C., followed by adding HATU (372.9 g, 2.0 eq.) portion wise over 30 min. After stirring for 10 min, a solution of DIPEA (238.9 g, 5.0 eq.) in DMF (1.1 L, 5.0 volume) was added over 45 min. Subsequently, the reaction mass was warmed to 25-30° C. with stirring. After stirring for 1 hr, HPLC analysis indicated that >99% of compound 3-2 was consumed. The reaction mixture was poured into water (38.0 L) and the mixture was extracted with DCM (10.0 L×3, 45.0 volume). The combined organic extracts were washed with water (10.0 L×3, 45.0 volume) and saturated brine (10 L, 45.0 volume) and dried over anhydrous Na₂SO₄. The solvent was removed at 40-45° C. under vacuum (600 mmHg) and the residue was purified by column chromatography on silica gel using DCM and MeOH as the eluent to give compound 3-3 (1.52 kg, 47% yield) as off white solid with a purity of >97.0% determined by HPLC analysis. LC-MS (ESI): m/z 763.4 [M+H]⁺. ¹H NMR (400 MHz, d⁶-DMSO): δ 8.60 (s, 1H), 8.29 (s, 1H), 8.20 (s, 1H), 8.09-8.14 (m, 2H), 7.99-8.05 (m, 2H), 7.86-7.95 (m, 3H), 7.20-7.21 (m, 2H), 5.24-5.33 (m, 2H), 4.06-4.18 (m, 4H), 3.83 (m, 2H), 3.53 (m, 6H), 2.26-2.55 (m, 10H), 0.85 (m, 6H), 0.78 (m, 6H) ppm. The transformation of 3-2 to 3-3 (Compound I) can be achieved via a range of conditions. One of these conditions is described below.

A reactor was charged with N-Moc-Valine (37.15 g, 0.211 mol), acetonitrile (750 mL) and DIPEA (22.5 g). The reaction mixture was agitated for 10 min and HOBT (35.3 g 0.361 mole) and EDCI (42.4 g, 0.221 mole) were added while keeping temperature <2° C. The reaction mixture was agitated for 30 min and DIPEA (22.5 g) and compound 3-2 (48.0 g, 0.092 mole) was added slowly to reactor over 30 min to keep temperature <3° C. The reaction mixture was agitated 4 hrs at 20-25° C., and sample was submitted for reaction completion analysis by HPLC (IPC specification: <1.0% area 3-2 remaining). At the completion of reaction as indicated by HPLC analysis, isopropyl acetate (750 mL) was added to the reactor and stirred for 10 min. The organic layer (product layer) was washed with brine (300 mL×2) and 2% NaOH (200 mL). The organic solution was filtered through a silica gel pad to remove insoluble material. The silica gel pad was washed with isopropyl acetate and concentrated under vacuum (400 mm/Hg) to a minimum volume. The crude product was purified by column chromatography on silica gel using ethyl acetate and methanol as eluent to give compound 3-3 (38.0 g, 65% yield) with purity of >95%. LC-MS (ESI): m/z 763.4 [M+H]⁺.

Step 4. Compound 3-3 (132.0 g, 1.0 eq.) and ethanol (324.0 mL, 2.0 volume) were charged into a 10 L four-necked round bottom flask under nitrogen atmosphere. After stirring for 15 min, the suspension was cooled to 5-10° C., to it was added 2.0 N HCl in ethanol (190 mL, 1.5 volume) over 30 min. The resulting solution was allowed to warm to 25-30° C. Acetone (3.96 L, 30.0 volume) was added over 90 min in to cause the slow precipitation. Next, the suspension was warmed to 60° C. and another batch of acetone (3.96 L, 30.0 volume) was added over 90 min. The temperature was maintained at 55-60° C. for 1 hr, and then allowed to cool to 25-30° C. After stirring at 25-30° C. for 8-10 hrs, the mixture was filtered. The solid was washed with acetone (660.0 mL, 5.0 volume) and dried in a vacuum tray drier at 50-55° C. for 16 hrs to give the di-HCl salt of compound 3-3 (compound I) (101 g, 71% yield) as pale yellow solid with a purity of >96.6% determined by HPLC analysis.

### Preparation of N-Moc-L-Valine

N-Moc-L-Valine is available for purchase but can also be made. Moc-L-Valine was prepared by dissolving 1.0 eq of L-valine hydrochloride in 2-methyltetrahydrofuran (2-MeTHF)/water containing sodium hydroxide and sodium carbonate, and then treating with 1.0 eq of methyl chloroformate at 0-5° C. for 6 hr. The reaction mixture was diluted with 2-MeTHF, acidified with HCl, and the organic layer was washed with water. The 2-MeTHF solution is concentrated and the compound is precipitated with n-heptane. The solid was rinsed with 2-MeTHF/n-heptane and dried in vacuo to give N-Moc-L-Valine in 68% yield.

### Crystallization of Compound I to Yield Form A

### Compound I Salt Formation and Crystallization

### Example 1

Ethanol (3.19 L, 1.0 volume, 200 proof) was charged to the 230-L glass lined reactor under nitrogen atmosphere. Free base form of compound 3-3 (3.19 kg, 4.18 mol) was added to the flask with stirring, stir continued for an additional 20 to 30 min. To the thick solution of 3-3 in ethanol was added slowly 2.6 N HCl in ethanol (3:19 L, 1.0 volume) to the above mass at 20-25° C. under nitrogen atmosphere. The entire mass was stirred for 20 min at rt, and then heated to 45-50° C. Acetone (128.0 L, 40.0 volume) was added to the above reaction mass at 45-50° C. over a period of 3-4 hrs before it was cooled to ˜25° C. and stirred for ˜15 hrs. The precipitated solid was collected by filtration and washed with acetone (6.4 L×2, 4.0 volume), suck dried for 1 hr and further dried in vacuum tray drier at 40-45° C. for 12 hrs. Yield: 2.5 kg (71.0% yield), purity by HPLC: 97.70%, XRPD: amorphous.

Isopropyl alcohol (7.5 L, 3.0 volume) was charged to a 50.0 L glass reactor protected under a nitrogen atmosphere. The amorphous di-HCl salt of 3-3 (2.5 kg) was added to the above reactor with stirring. The entire mass was heated to 60-65° C. to give a clear solution. Stir continued at 65±2° C. for ˜15 hrs, solid formation started during this time. The heating temperature was lowered to ˜50° C. over a period of 3 hrs, methyl tertiary butyl ether (12.5 L, 5.0 volume) was added to the above mass slowly over a period of ˜3 hrs with gentle agitation. The above reaction mass was further cooled to 25-30° C. over 2-3 hrs. The solid was collected by filtration, washed with 10.0% isopropyl alcohol in methyl tertiary butyl ether (6.25 L, 2.5 volume), suck dried for 1 hr and further dried in a tray drier at 45-50° C. under vacuum (600 mm/Hg) for 70-80 hrs. Yield: 2.13 kg (85.0% recovery, 61.0% yield based on the input of compound free base 3-3), purity by HPLC: 97.9%.

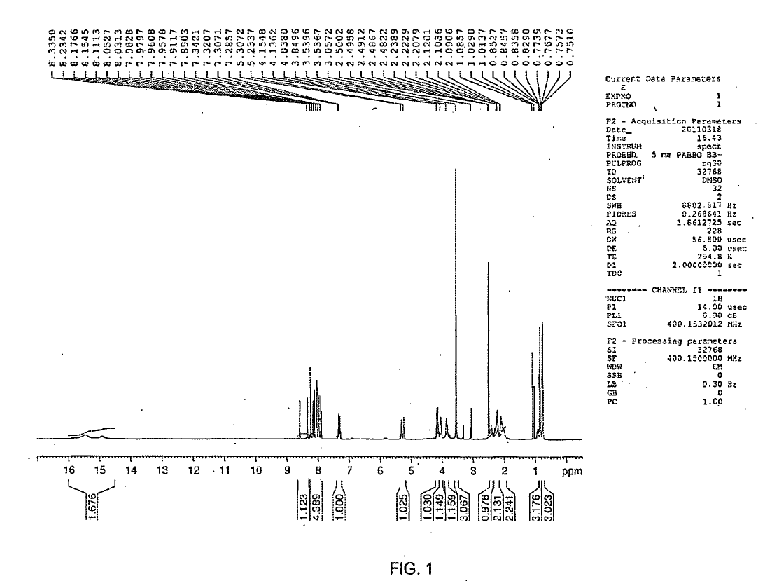

FIG. 1: ¹H NMR (500 MHz, d⁶-DMSO): δ 15.6 (bs, 2H), 14.7 (bs, 2H), 8.58 (s, 1H), 8.35 (s, 1H), 8.25 (s, 1H), 8.18 (d, J=8.7 Hz, 1H), 8.13 (s, 1H), 8.06 (d, J=8.6 Hz, 1H), 8.04 (s, 1H), 8.00 (s, 1H), 7.98 (d, J=8.7 Hz, 1H), 7.91 (d, J=8.6 Hz, 1H), 7.36 (d, J=8.6 Hz, 1H), 7.33 (d, J=8.6 Hz, 2H), 5.31 (m, 1H), 5.26 (m, 1H), 4.16 (d, J=7.7 Hz, 1H), 4.04 (m, 2H), 3.87 (m, 2H), 3.55 (s, 6H), 2.42 (m, 2H), 2.22-2.26 (m, 4H), 2.07-2.14 (m, 4H), 0.86 (d, J=2.6 Hz, 3H), 0.84 (d, J=2.6 Hz, 3H), 0.78 (d, J=2.2 Hz, 3H), 0.77 (d, J=2.2 Hz, 3H), 3.06 (s, OMe of MTBE), 1.09 (s, t-Bu of MTBE), 1.03 (d, 2Me of IPA) ppm.

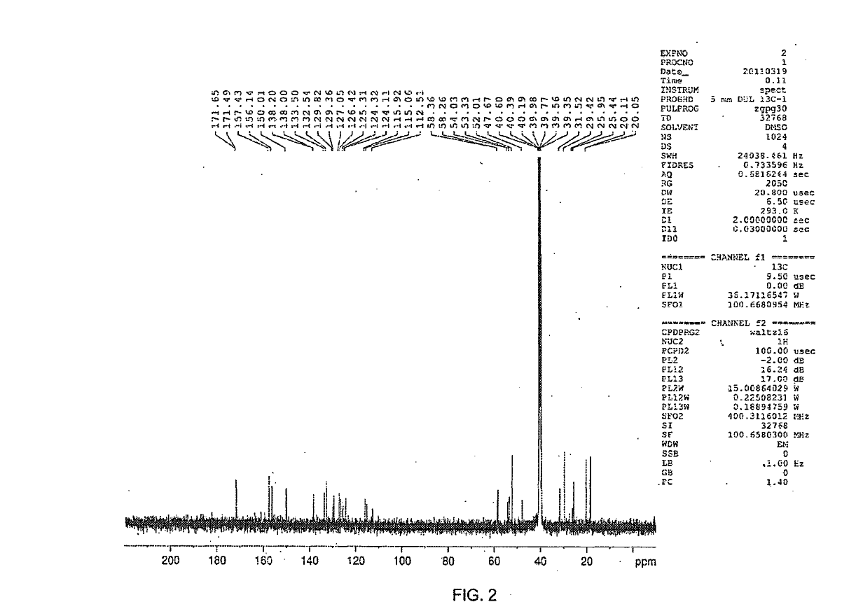

FIG. 2: ¹³C NMR (500 MHz, d⁶-DMSO): δ 171.6, 171.5, 157.4, 156.1, 150.0, 138.2, 138.0, 133.5, 132.5, 131.3, 129.8, 129.4, 128.0, 127.0, 126.4, 125.6, 125.3, 124.4, 124.2, 115.8, 115.0, 112.5, 58.37, 58.26, 54.03, 53.34, 52.00 (2 carbons), 47.71 (2 carbons), 31.52, 31.47, 29.42 (2 carbons), 25.94, 25.44, 20.13, 20.07, 18.37, 18.36 ppm.

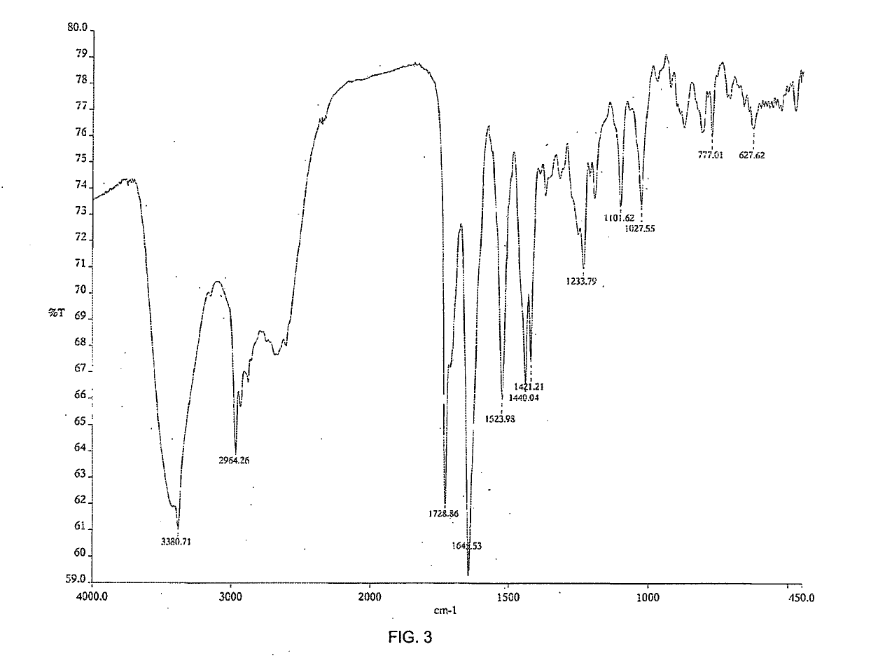

FIG. 3: FT-IR (KBr pellet): 3379.0, 2963.4, 2602.1, 1728.4, 1600.0, 1523.4, 1439.7, 1420.6, 1233.2, 1193.4, 1100.9, 1027.3 cm⁻¹.

Elemental Analysis: Anal. Calcd for C₄₂H₅₂Cl₂N₈O₆: C, 60.35; H, 6.27; N, 13.41; Cl, 8.48. Found C, 58.63; H, 6.42; N, 12.65; Cl, 8.2.

FIG. 4: DSC: peak value, 256.48° C. Water content by Karl Fischer=1.0%.

FIG. 5: XRPD: crystalline. The peaks of FIG. 5 are listed in FIG. 6. The procedure for the XRPD is provided in Compound I, Example 2.

### Compound I Crystallization Condition

### Example 2

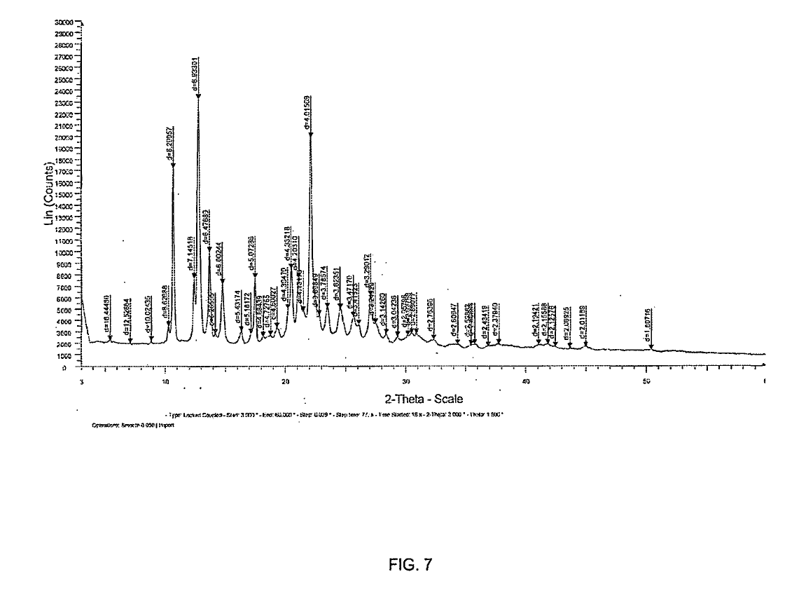

A sample of the amorphous di-HCl salt of compound 3-3 (2.0 g) was dissolved in 6.0 mL of isopropyl alcohol (3.0 volume) with stirring and heating at 65° C. The solution was stirred at this temperature for 20 hrs, crystallization initiated during this time. The mass was cooled to ˜50° C. and maintained at this temperature for 3 hrs before 6.0 mL of IPA (3.0 volume) was added over a period of 1 hr. The temperature was kept at 50° C. for another hour before it was filtered, and the solid was washed with chilled IPA 6.0 mL (3.0 volume), and was dried in vacuum tray drier at 40-45° C. for 10 hrs. Yield: 1.0 g in 50.0%. The crystallinity of the sample was analyzed by XRPD with a Broker D-8 Discover diffractometer and Bruker's General Detector System (GADDS, v. 4.1.20) using an incident microbeam of Cu Kα radiation was produced using a fine-focus tube (40 kV, 40 mA), a Göbel mirror, and a 0.5 mm double-pinhole collimator. Diffraction patterns were collected using a Hi-Star area detector located 15 cm from the sample and processed using GADDS. The intensity in the GADDS image of the diffraction pattern was integrated using a step size of 0.04° 2θ. The integrated patterns display diffraction intensity as a function of 2θ. The data acquisition parameters are displayed in the resulting spectrum at FIG. 7 and the peaks of FIG. 7 are provided in FIG. 8.

### Compound I Crystallization Condition

### Example 3

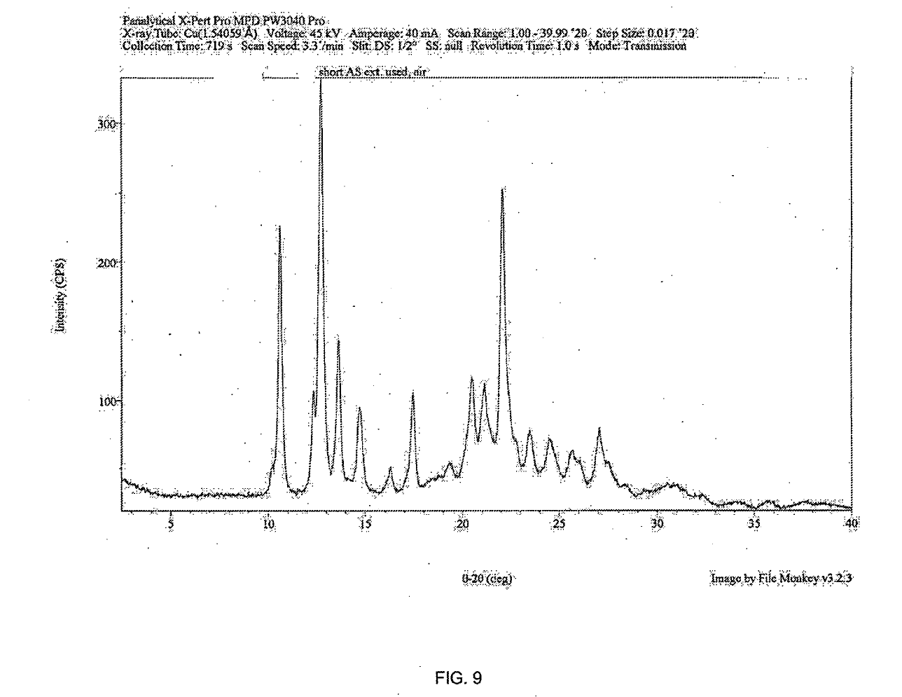

Approximately 2 g of amorphous Compound I was dried overnight under vacuum and then added to 6 mL of IPA in a 50 mL round bottom flask (˜344 mg/mL). The flask was attached to a cold water condenser and the solution was heated at ˜60° C. in an oil bath while stirred under nitrogen for 20 hrs. Off-white solids precipitated overnight. The solution was cooled from ˜60° C. to ambient temperature at a rate of ˜6° C./hr to 45° C.; ˜12° C./hr from 45° C. to 32.5° C. and ˜24° C./hr from 32.5° C. to rt. At ambient temperature the cold water condenser and nitrogen stream were removed and MTBE was added dropwise for ˜30 minutes for a total of 10 mL (IPA/MTBE=3/5 (v/v)). The solution was stirred overnight, solids were collected by vacuum filtration and the 50 mL flask was washed with ˜5 mL of IPA. Solids were dried in vacuo at ambient temperature for ˜2.5 hrs and analyzed by XRPD (see Procedure for PANalvtical X'PERT Pro MPD Diffractometer). Yield of Form A was ˜88%. The data acquisition parameters are displayed in the resulting spectrum at FIG. 9 including the divergence slit (DS) before the mirror and the incident-beam anti scatter slit (SS). Form A.

### Compound I Crystallization

### Example 4

Form A was also obtained by slurring a sample of amorphous di-HCl salt of compound 3-3 in a mixture of methanol and diethyl ether (in 1:4 ratio) at elevated temperature (˜60° C.) over 2 days.

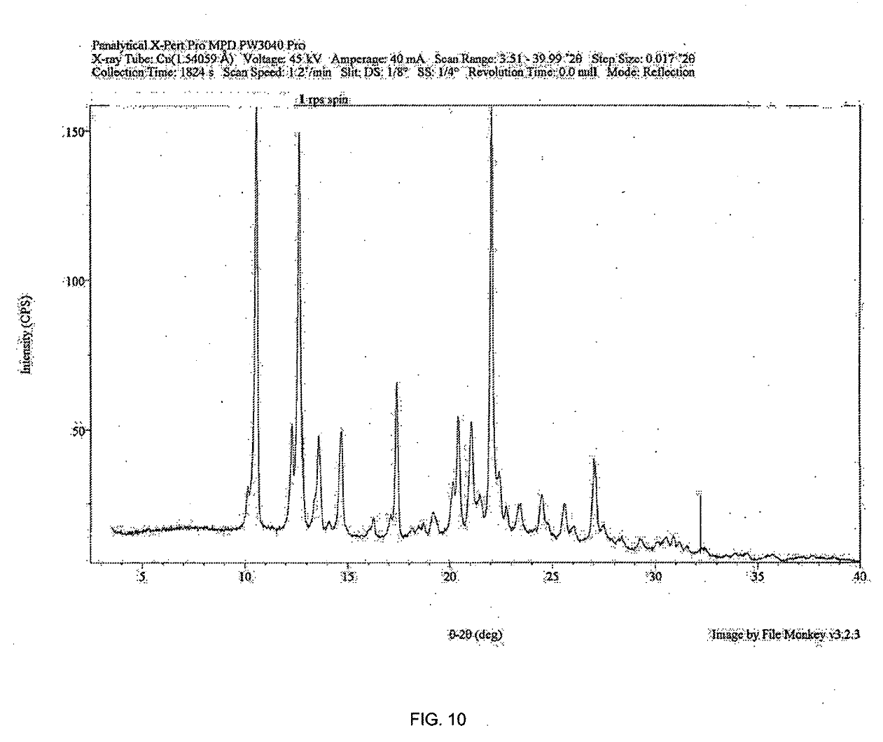

XRPD was acquired with PANalytical X'PERT Pro MPD Diffractometer (see procedure above). The data acquisition parameters for each pattern are displayed in the resulting spectrum at FIG. 10 including the divergence slit (DS) and the incident-beam antiscatter slit (SS).

Observed peaks for FIG. 10 are provided in Table 1 in Appendix A and Prominent Peaks for FIG. 10 are provided in Table 2 in Appendix A. The location of the peaks along the x-axis (° 2θ) in both the figures and the tables were automatically determined using PATTERNMATCH™ software v. 3.0.4 and rounded to one or two significant figures after the decimal point based upon the above criteria. Peak position variabilities are given to within ±0.2° 2θ based upon recommendations outlined in the United States Pharmacopeia, USP 33 reissue, NF 28, <941>, R-93, Oct. 1, 2010 discussion of variability in x-ray powder diffraction.

The sample was also analyzed by proton NMR which identified the API and trace amounts of Et₂O. The solution ¹H NMR spectrum was acquired at ambient temperature with a Varian^{UNITY}INOVA-400 spectrometer at a ¹H Larmor frequency of approximately 400 MHz. The sample was dissolved in d⁶-DMSO containing TMS. The results and sample acquisition parameters are shown at FIG. 11.

### Compound I Crystallization

### Example 5

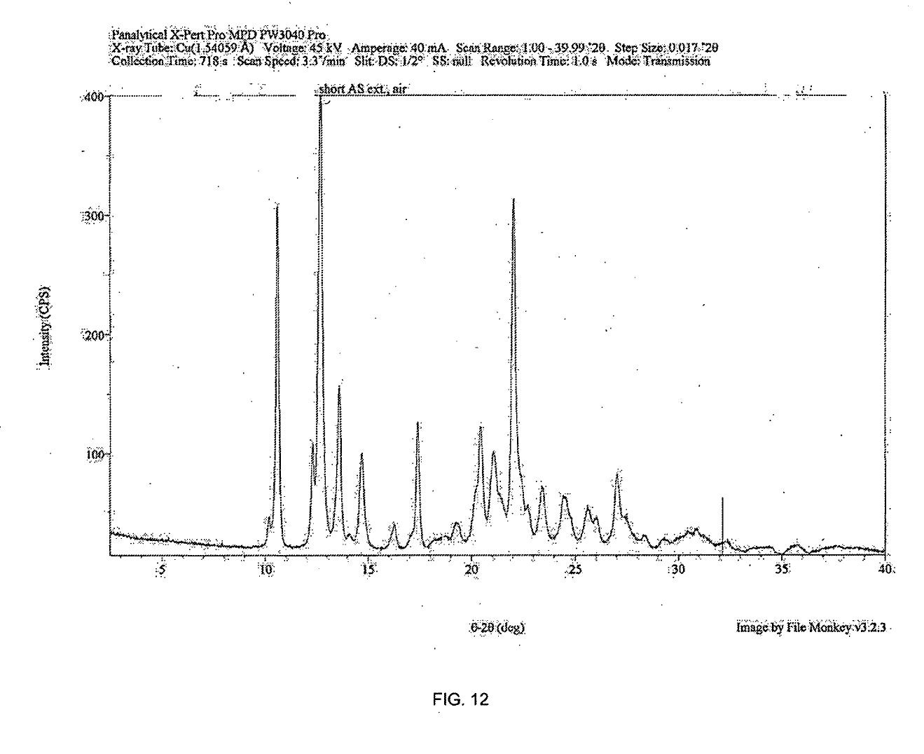

Form A was also obtained by the following procedure. A 2.0 g sample of the amorphous diHCl salt was dissolved in 6.0 mL of IPA with heating. The mixture was maintained 65° C. for ˜20 hrs with gentle stirring. The solid came out and was filtered while hot and vacuum dried to give Form A in ˜25% recovery yield. XRPD patterns were collected with a PANalytical X'Pert PRO MPD diffractometer (see procedure above). The data acquisition parameters are displayed in the resulting spectrum at FIG. 12 including the divergence slit (DS) before the mirror and the incident-beam anti scatter slit (SS).

The sample was also analyzed by proton NMR which identified the API, IPA (0.2 moles, 1.3% by weight) and water per the NMR procedure given above. The results and sample acquisition parameters are shown at FIG. 13.

The sample was also analyzed by modulated differential scanning calorimetry and thermogravimetrically by the procedures described above.

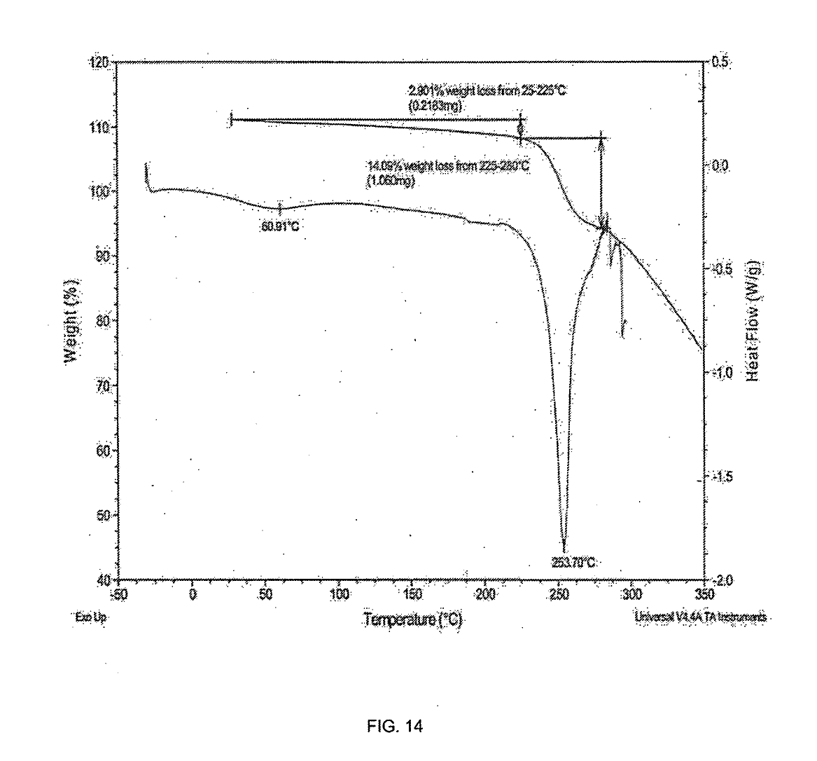

The resulting DSC curve and thermogram are shown in FIG. 14.

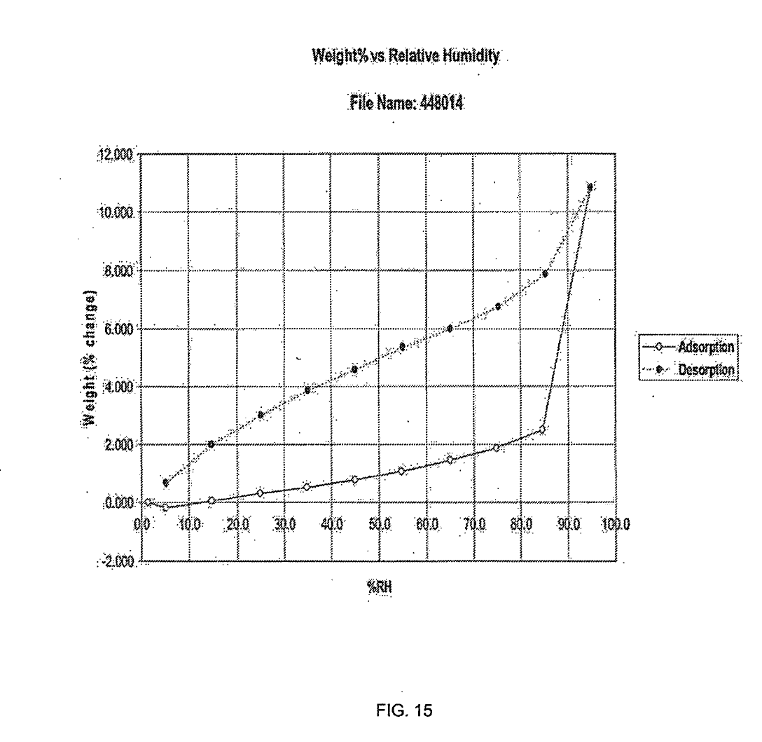

Moisture sorption/desorption data were collected for the sample on a VTI SGA-TOO Vapor Sorption Analyzer. NaCl and PVP were used as calibration standards. Samples were vacuum dried prior to analysis. Sorption and desorption data were collected over a range from 5 to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples. FIG. 15 illustrates the graphed Weight % vs. Relative Humidity. Table 3 in Appendix A shows collected data.

### Compound I Crystallization

### Example 6

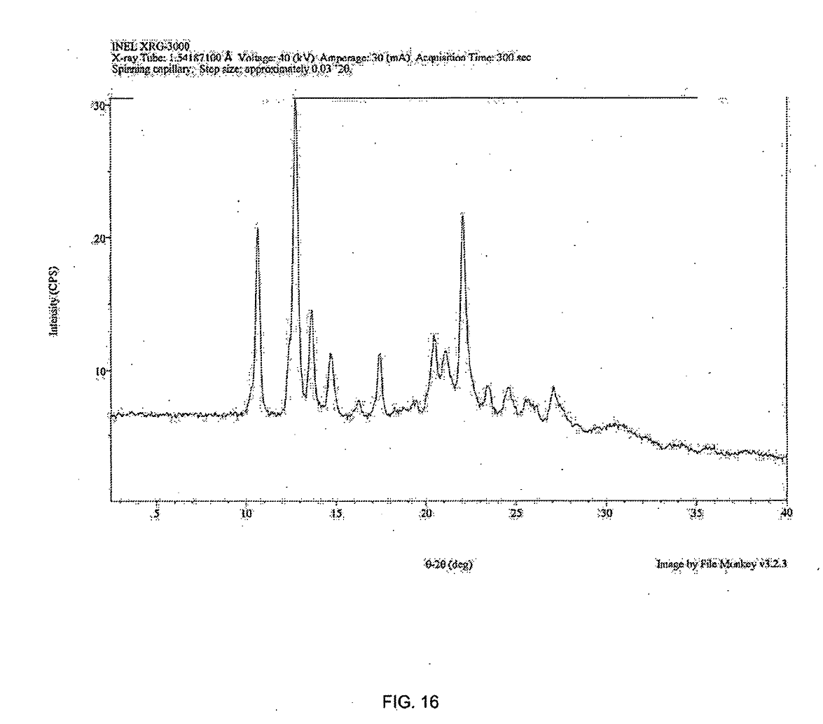

Form A was also crystallized from IPA/MTBE (1/1 (v/v)) and air dried. XRPD patterns were collected with an Inel XRG-3000 diffractometer using the procedure described above. The data-acquisition parameters are displayed above the spectrum in FIG. 16.

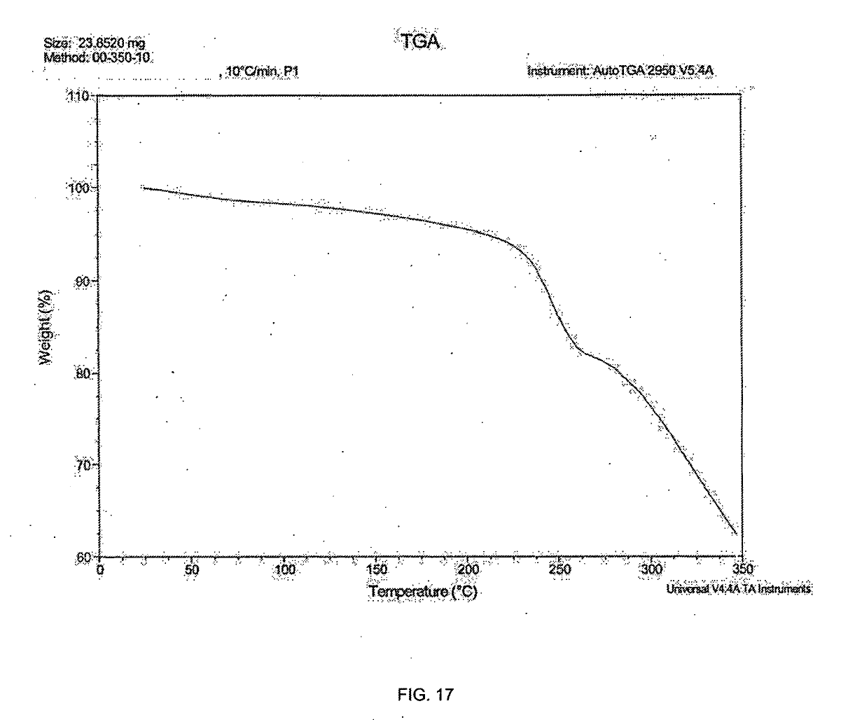

The sample was also analyzed thermogravimetrically. The resulting thermogram is FIG. 17.

The sample was also subjected to Karl Fischer analysis. Coulometric Karl Fischer (KF) analysis for water determination was performed using a Mettler Toledo DL39 KF titrator. A blank titration was carried out prior to analysis. The sample was prepared under a dry nitrogen atmosphere, where 90-100 mg of the sample were dissolved in approximately 1 mL dry Hydranal-Coulomat AD in a pre-dried vial. The entire solution was added to the KF coulometer through a septum and mixed for 10 seconds. The sample was then titrated by means of a generator electrode, which produces iodine by electrochemical oxidation: 2I⁻ →I₂+2e⁻. Two replicates were obtained. The obtained data is shown below in Tables 4 and 5 attached in Appendix A.

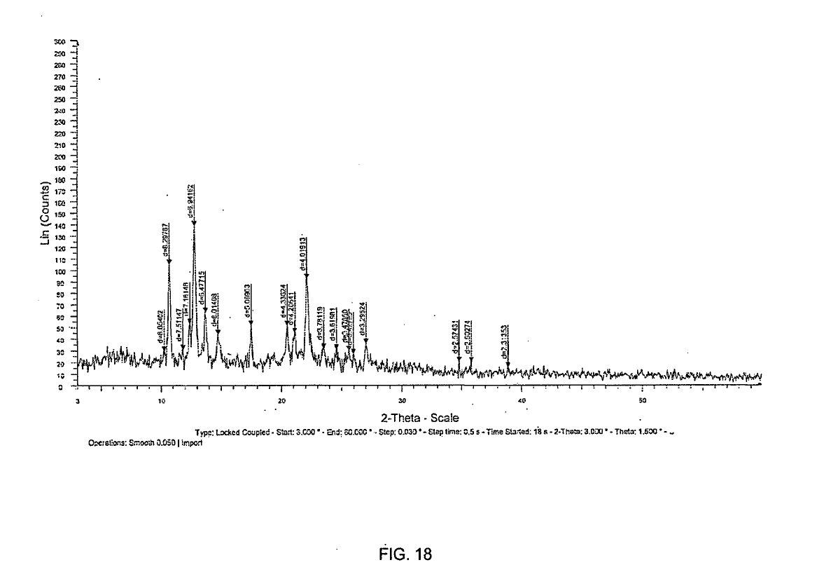

Another sample crystallized from IPA/MTBE provided XRPD pattern shown in FIG. 18. The XRPD procedure is the same as for Compound I, Example 2. The list of peaks is provided in FIG. 19.

### Compound I Crystallization

### Example 7

Compound 3-3 (free base, 1.71 kg) and ethanol (8.90 kg) were charged to a reactor vassel equipped with a condenser and distillation set-up. To it was added with agitation a sufficient volume of an HCl solution in ethanol (1.25 M, ˜3.5 kg) and until the measured pH<3, agitation continued for an additional 30 min. The solvent was distilled off in vacuo at <40±5° C. Methanol (20 kg) was charged to the reactor, after mixing, the solvent was again distilled off (˜18 kg) in vacuo at <40° C. The solvent chasing process was repeated once more with methanol, and once with IPA (15 kg). Fresh IPA (14 kg) was charged to the reactor again, and partially distilled off (˜7 kg) in vacuo at <40±5° C. The content of the reactor was heated to 65±5° C. and maintained at this temperature for 47 hrs for crystallization to take place. The mass was gradually cooled down to 25±5° C. over a 6 hrs period, agitation continued at this temperature for another 20 hrs. The solid product was isolated by filtration to give the first crop.

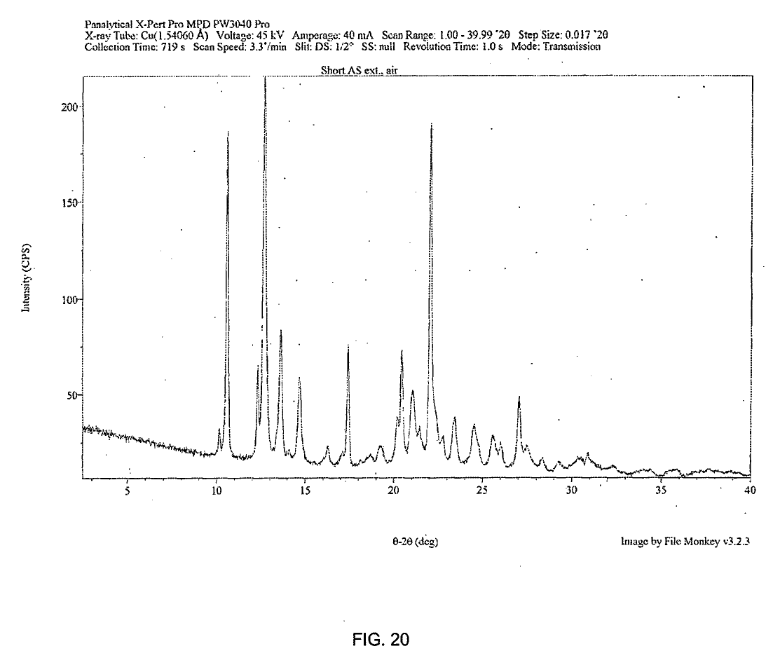

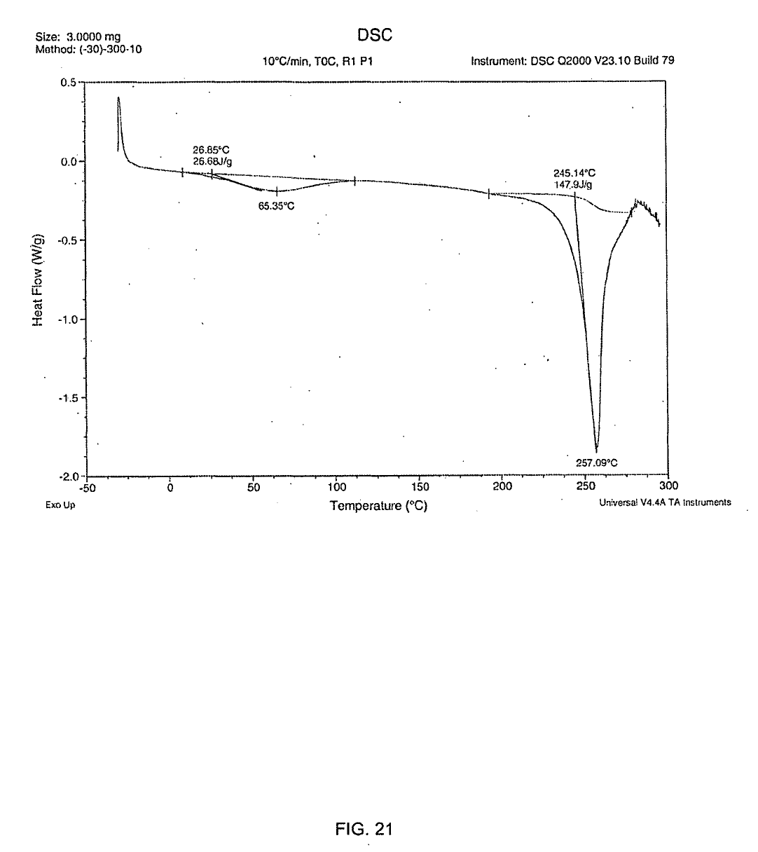

The filtrate was transferred back to the reactor aided with IPA (2.5 kg×2). IPA was partially (˜6 kg) distilled off in vacuo at <40±5° C. The mixture was heated to 65±5° C. for 60 hrs while with gentle agitation (90 RPM), cooled down to 25±5° C. over 6 hrs and for another 20 hrs. Additional solid product was collected by filtration and rinsed with cold IPA to get the second crop. The two crops were combined and dried under vacuum and at 40±5° C. to remove IPA, A total of 1.294 kg product was obtained, and the crystalline Form A was confirmed by XRPD (FIG. 20). Thermogravimetric analysis is provided in FIG. 21.

To upgrade the HPLC purity, this material was recrystallized using similar procedures.

The salt product from above (559 g) and methanol (3.0 kg) were charged to a reactor equipped with a distillation set-up. Methanol was distilled off (˜2.8 kg) in vacuo at <40° C. IPA (2.86 kg) was added and distilled off (˜2.46 kg) in vacuo at <40±5° C. Fresh IPA (3.58 kg) was added, and was partially distilled off (2.43 kg) in vacuo at 40±5° C. The content was heated at 65±5° C. for 45 hrs while with gentle agitation (90 RPM), cooled down to 25±5° C. over 9 hrs and for another 32 hrs. The solid was collected filtration and dried in a vacuum oven with temperature at 40±5° C. over 2 days to a constant weight. 493 g of Compound I was obtained and was further characterized.

### Stressing of Form A

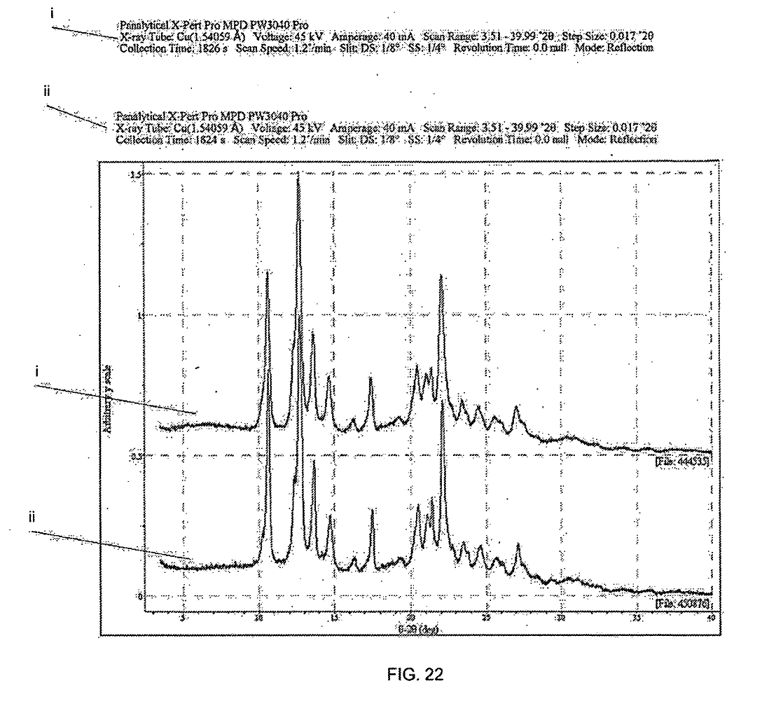

Form A samples were stressed at ˜40° C./˜75% relative humidity (RH) for 25-27 days. The samples were added to glass vials and then placed uncapped in jars containing saturated salt solutions. The jars were sealed and placed in an oven. After 25 days, XRPD analysis (shown in FIG. 22) indicated that the material remained Form A. FIG. 22 displays a spectrum of Form A prior to stressing on top (i) and after stressing below (ii). XRPD patterns for this sample were collected with a PANalytical X'Pert PRO MPD diffractometer using an incident beam of Cu Kα radiation produced using a long, fine-focus source and a nickel filter. The diffractometer was configured using the symmetric Bragg-Brentano. Prior to the analysis, a silicon specimen (NIST SRM 640d) was analyzed to verify the Si 111 peak position. A specimen of the sample was packing into a nickel-coated copper well. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for the two spectra are displayed at the top of FIG. 22

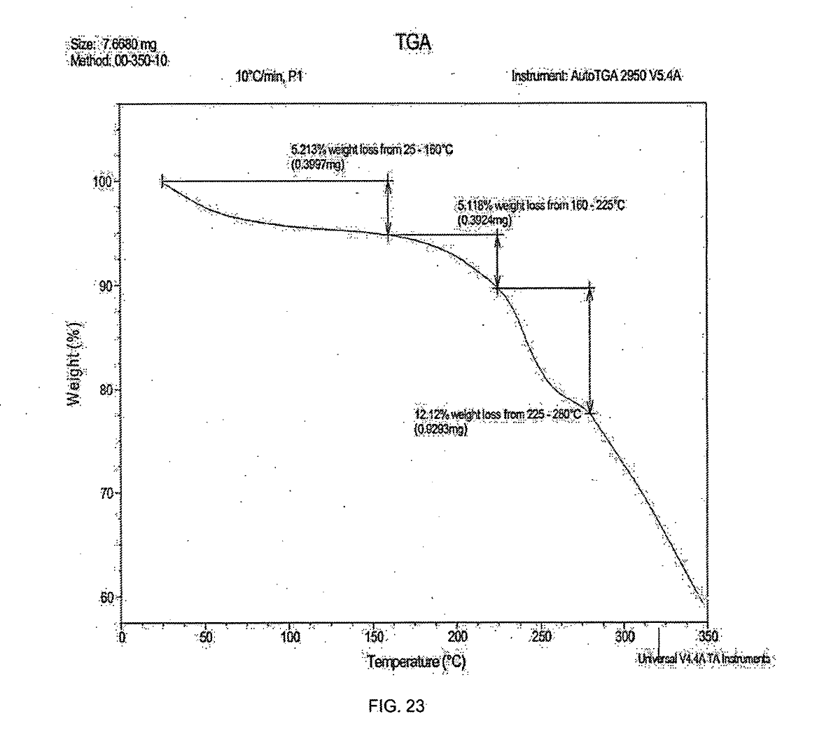

After 27 days, thermogravimetric analysis (shown in FIG. 23) displayed ˜10% weight loss (equivalent to 5 moles of water) from 25-225° C. This increase compared with the unstressed material indicated that Form A is hygroscopic at high RH. TG′analysis was performed using a TA Instruments Q5000 IR and 2950 thermogravimetric analyzers. Temperature calibration was performed using nickel and Alumel™. Each sample was placed in an aluminum pan. Samples ran on TA Instruments 2950 were left uncapped and samples ran on Q5000 was hermetically sealed, the lid pierced, then inserted into the TG furnace. The furnace was heated under nitrogen. The sample was heated from 0° C. to 350° C., at 10° C./min.

### Solubility of Form A

Aliquots of various solvents were added to measured amounts of Form A with agitation (typically sonication) at ambient or elevated temperatures until completedissolution was achieved, as judged by visual observation. Solubility estimates performed by aliquot addition, indicated that Form A is poorly soluble in IPA and IPA/MTBE (2/1 (v/v)) mixtures at ambient and elevated temperatures. Samples were left to slurry at ambient and elevated temperatures for several days; however, no further dissolution was observed. Furthermore, Form A is significantly more soluble in IPA/water (95/5 (v/v)) at ambient temperature compared to pure IPA (33 mg/mL compared to less than 3 mg/mL). Results are shown in Table 7 in Appendix A.

### Example

### Synthesis of Compound II (Aka Di-HCl Salt of Compound 4-3)

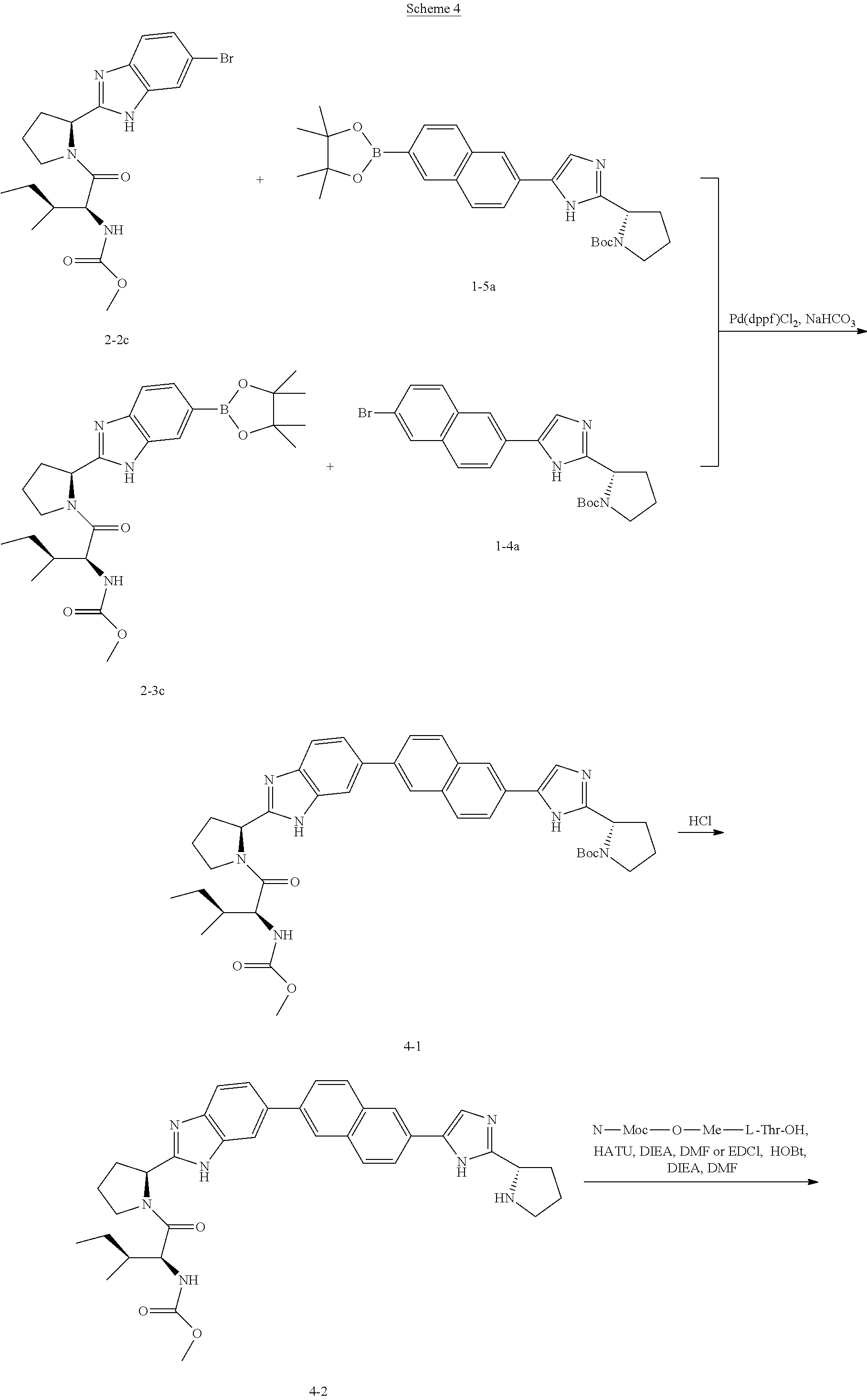

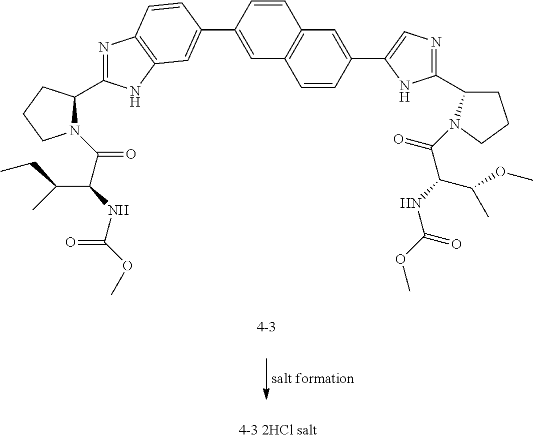 [SMILES: CC[C@H](C)[C@H](NC(=O)OC)C(=O)N1CCC[C@H]1c1nc2ccc(-c3ccc4cc(-c5cnc([C@@H]6CCCN6C(=O)[C@@H](NC(=O)OC)[C@@H](C)OC)[nH]5)ccc4c3)cc2[nH]1]

Step 1. Referring to Scheme 4, following the procedure described previously for the synthesis of compound 3-1 in Scheme 3 (in Synthesis of Compound I) and replacing 2-2a with 2-2c, compound 4-1 was obtained (3.4 kg, 54% yield) as off-white solid with a purity of >94.0% determined by HPLC analysis. LC-MS (ESI) m/z 720.4 [M+H]⁺. Alternatively, compound 4-1 can be obtained by following the same Suzuki coupling condition and replacing compound 1-5a and 2-2c with compound 1-4a and 2-3c.

Step 2. Following the procedure described previously for the synthesis of compound 3-2 in Scheme 3 and replacing compound 3-1 with 4-1, compound 4-2 was obtained (2.2 kg, 85% yield) as yellow solid with a purity of >95.0% determined by HPLC analysis. LC-MS (ESI) m/z 620.3 [M+H]⁺.

Step 3. Following the procedure described previously for the synthesis of compound 3-3 in Scheme 3 and replacing compound 3-2 with 4-2, compound 4-3 was obtained (65 g, 57% yield) as pale yellow solid with a purity of >92% determined by HPLC analysis. LC-MS (ESI) m/z 793.4 [M+H]⁺.

Step 4. HCl salt formation and crystallization. Compound 4-3 (free-base, 5.0 g) was dissolved in 15.0 mL of MeOH at 65° C. with stirring. After adding 2.5 N HCl in EtOH (6.3 mL), the resulting clear solution was stirred at 65° C. for 15 min. Next, acetone (150 mL) was added dropwise over a period of 1.5 hrs until the cloudy point was reached. The suspension was kept stirring at 65° C. for 1 hr and then slowly cooled down (˜5° C./30 min) to rt (˜30° C.). After stirring at rt overnight, the solid was collected by filtration, washed with acetone (3×5 mL) and dried in vacuo to give the di-HCl salt of compound 4-3 (Compound II) (4.4 g, 80% yield) as pale yellow solid. The solid was further characterized and was shown to be crystalline. ¹H NMR (500 MHz, d⁶-DMSO): δ 15.5 (bs, 2H), 15.0 (bs, 2H), 8.63 (s, 1H), 8.35 (s, 1H), 8.25 (s, 1H), 8.17 (d, J=7.8 Hz, 1H), 8.12 (s, 1H), 8.08 (d, J=1.5 Hz, 1H), 8.04 (s, 1H), 7.99 (s, 1H), 7.98 (d, J=8.5 Hz, 1H), 7.92 (d, J=7.2 Hz, 1H), 7.39 (d, J=8.6 Hz, 1H), 7.11 (d, J=8.6 Hz, 2H), 5.31 (m, 1H), 5.25 (m, 1H), 4.31 (m, 1H), 4.19 (m, 1H), 4.07 (m, 2H), 3.93 (m, 2H), 3.87 (m, 2H), 3.55 (s, 6H), 3.20 9s, 3H), 2.42 (m, 2H), 2.22-2.26 (m, 4H), 2.07-2.14 (m, 4), 1.81 (m, 1H0, 1.33 (m, 1H), 1.05 (d, J=2.6 Hz, 3H), 0.80 (m, 6H) ppm.

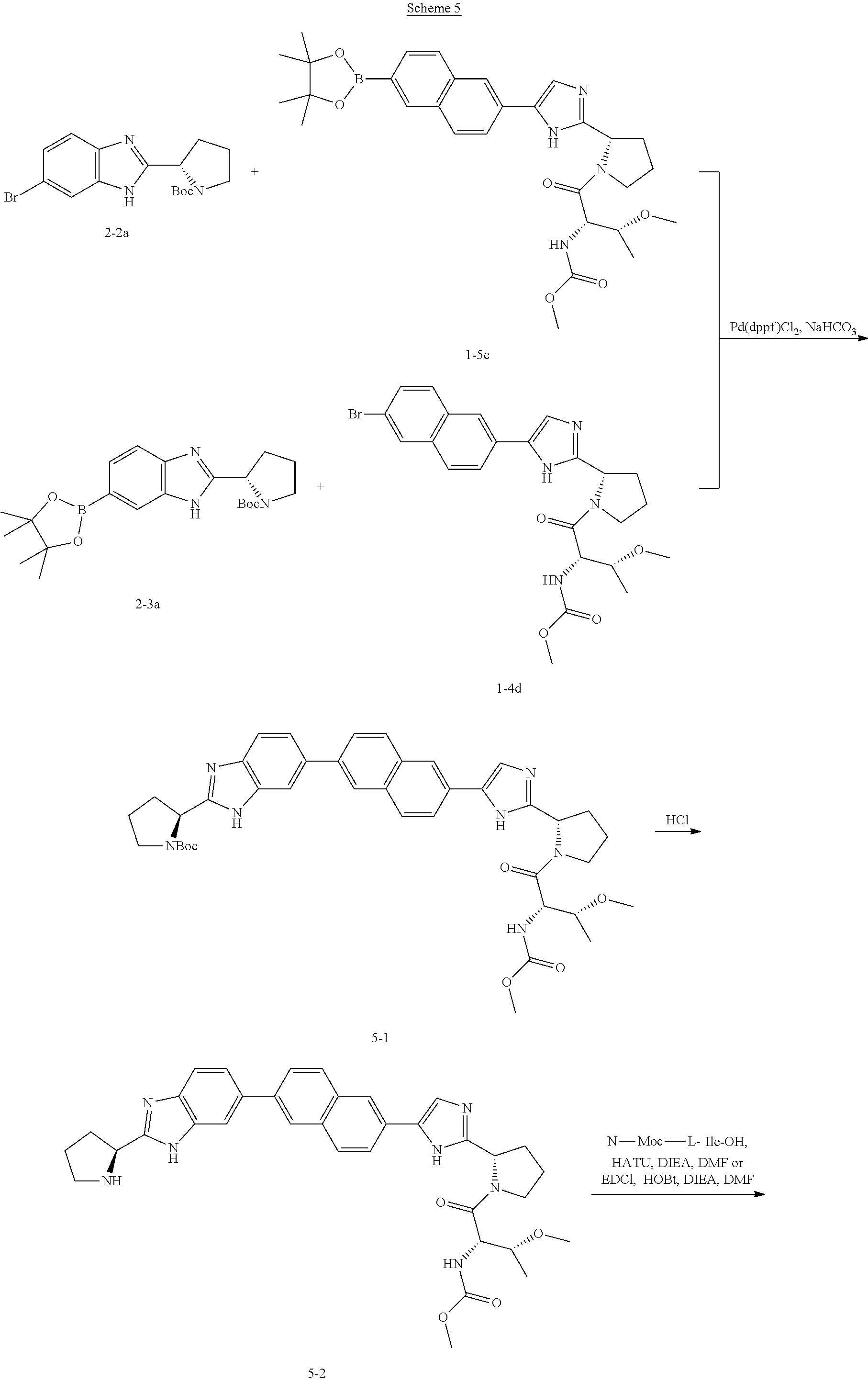

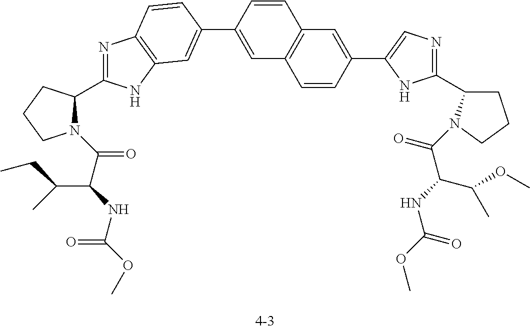 [SMILES: CC[C@H](C)[C@H](NC(=O)OC)C(=O)N1CCC[C@H]1c1nc2ccc(-c3ccc4cc(-c5cnc([C@@H]6CCCN6C(=O)[C@@H](NC(=O)OC)[C@@H](C)OC)[nH]5)ccc4c3)cc2[nH]1]

Step 1. Referring to Scheme 5, following the procedure as described for the synthesis of compound 3-1 in Scheme 3 and replacing compound 1-5a with 1-5c, compound 5-1 was obtained. LC-MS (ESI): m/z 722.4 [M+H]⁺. Alternatively, compound 5-1 can be obtained by using the same Suzuki coupling condition and replacing compounds 1-5c and 2-2a with compounds 1-4d and 2-3a.

Step 2. Following the same procedure as described for the synthesis of compound 3-2 in Scheme 3 and replacing compound 3-1 with 5-1, compound 5-2 was obtained. LC-MS (ESI): m/z 622.3 [M+H]⁺.

Step 3. Following the same procedure as described for the synthesis of compound 3-3 in Scheme 3 and replacing compound 3-2 with 5-2, compound 4-3 was obtained. LC-MS (ESI): m/z 793.4 [M+]⁺.

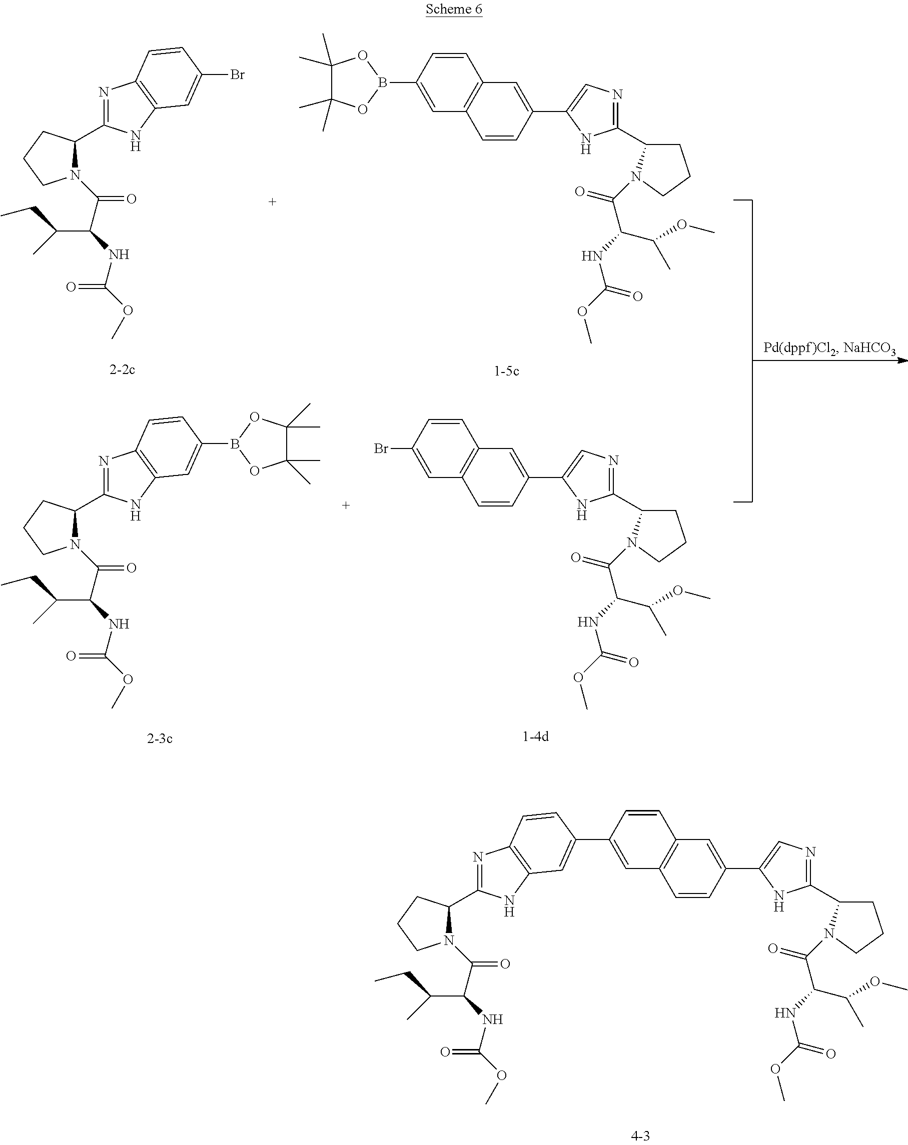

Compound 4-3 may be prepared by alternative routes, as those described in Schemes 6, 7 and 8.

Referring to Scheme 6, following the Suzuki coupling conditions for compounds 1-5a and 2-2a as described in Scheme 3, compound 4-3 was obtained by coupling of either compounds 1-5c and 2-2c or compounds 1-4d and 2-3c.

### Additional Syntheses of Compound 3-3

Following the approach to compound 4-3 as described in Scheme 4, compound 3-3 can be obtained by replacing either compound 2-2c with 2-2b or compound 2-3c with 2-3b and N-Moc-O-Me-L-Thr-OH with N-Moc-L-Val-OH.

Following the approach to compound 4-3 as described in Scheme 5, compound 3-3 can be obtained by replacing either compound 1-5c with 1-5b or compound 1-4d with 1-4c and N-Moc-L-Ile-OH with N-Moc-L-Val-OH.

Following the approach to compound 4-3 as described in Scheme 6, compound 3-3 is obtained by replacing either compound 2-2c with 2-2b and compound 1-5c with 1-5b or compound 2-3c with 2-3b and compound 1-4d with 1-4c.

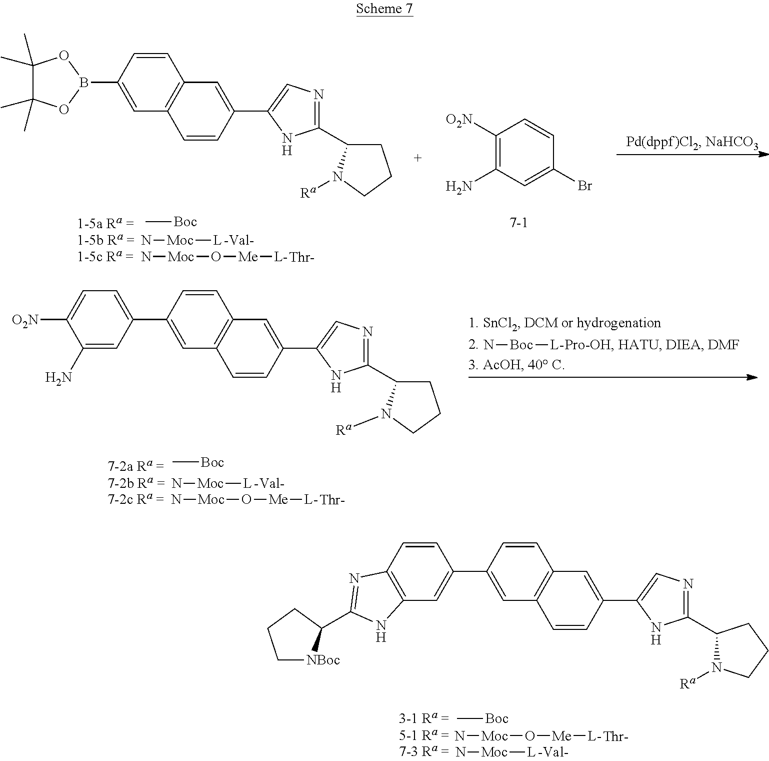

Step 1. Referring to Scheme 7, following the Suzuki coupling condition used for coupling compounds 1-5a and 2-2a as described in Scheme 3, compounds 7-2a, 7-2b and 7-2c are obtained, respectively, by coupling compound 7-1 with compounds 1-5a, 1-5b and 1-5c, respectively.

Step 2. Reduction of the —NO₂ group in compounds 7-2a, 7-2b and 7-2c, respectively, by typical hydrogenation (mediated by Pd/C, Pd(OH)₂, PtO₂ or Raney Ni, etc.) or other —NO₂ reduction conditions (such as SnCl₂/DCM or Zn/AcOH, etc.), followed by a two-step transformation as described for the synthesis of compound 2-2a from 2-1 in Scheme 2 give compounds 3-1, 5-1 and 7-1, respectively.

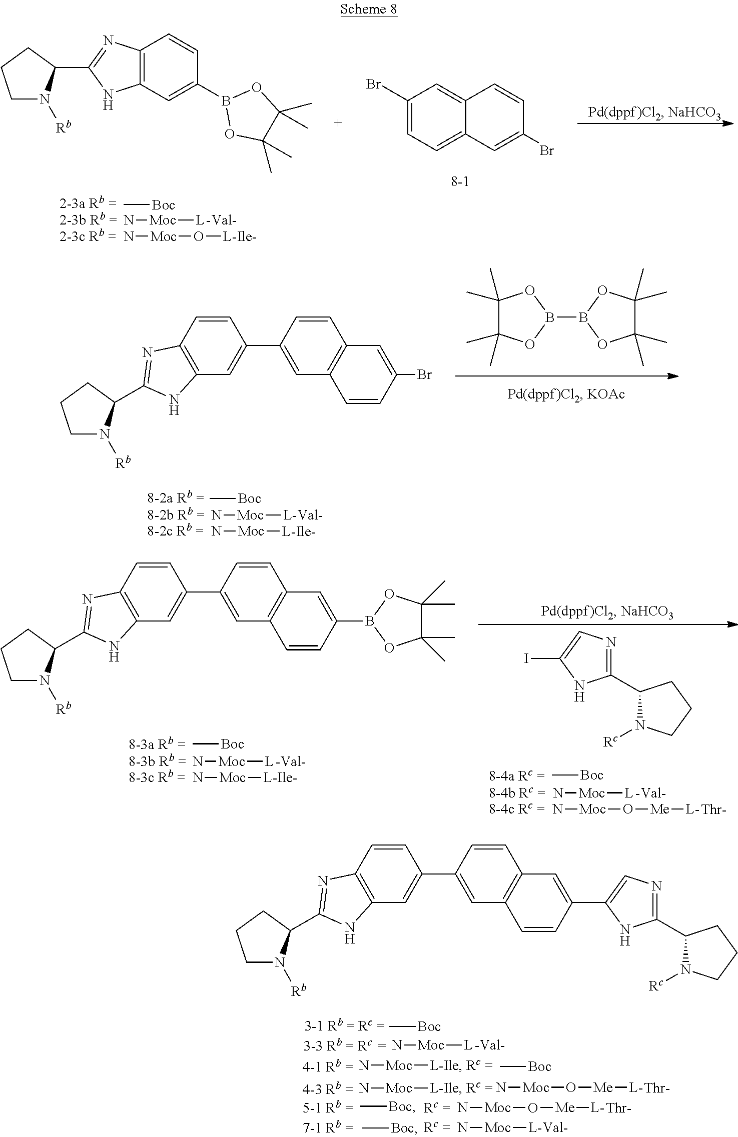

Step 1. Refer to Scheme 8. Following the Suzuki coupling condition used for coupling compounds 1-5a and 2-2a as described in Scheme 3, compounds 8-2a, 8-2b and 8-2c are obtained, respectively, by coupling compound 8-1 with compounds 2-3a, 2-3b and 2-3c, respectively.

Step 2. Following the condition used for converting compound 1-4a to 1-5a as described in Scheme 1, compounds 8-3a, 8-3b and 8-3c are obtained, respectively, by replacing compound 1-4a with compounds 8-2a, 8-2b and 8-2c, respectively.

Step 3. Following the Suzuki coupling condition used for coupling compounds 1-5a and 2-2a as described in Scheme 3, compounds 3-1, 3-3, 4-1, 4-3, 5-1 and 7-3 are obtained, respectively, by replacing compounds 1-5a and 2-2a with compounds 8-3a and 8-4a (WO2010065668), compounds 8-3b and 8-4a, compounds 8-3c and 8-4a, compounds 8-3c and 8-4b, compounds 8-3a and 8-4c, and compounds 8-3a and 8-4b, respectively.

### (f). Crystallization of Compound II to Yield Form I

### Compound II Crystallization

### Example 1

113.1 mg of Compound 4-3 (free base form of Compound II) was weighed into a vial and dissolved by 1 mL of methanol. 47.6 μL of 6 M H Cl was added with stirring at 60° C. Then the solution was evaporated under a stream of nitrogen.

To the vial, 1 mL of methanol was added at 60° C. with stirring. 8 mL Acetone was added. A clear solution formed. 1.9 mL of MTBE was added to cloud point. The sample was slowly cooled down to rt. Many particles precipitated out. The solid was collected by vacuum filtration, dried under reduced pressure. The yield was 88.2%. The resulting solid was analyzed by XRPD. XRPD patterns were obtained on a Bruker D8 Advance. A CuKa source (=1.54056 angstrom) operating minimally at 40 kV and 40 mA scans each sample between 4 and 40 degrees 2-theta. The spectrum is shown as line A in FIG. 24.

### Compound II Crystallization

### Example 2

106.0 mg of Compound 4-3 (free base form of Compound II) was weighed into a vial and dissolved by 1 mL of methanol. 44.6 μL of 6 M HCl was added with stirring at 60° C. Then the solution was evaporated under a stream of nitrogen.

To the vial, 1 mL of methanol was added at 60° C. with stirring. 8 mL Acetone was added. A clear solution formed. 2.2 mL of MTBE was added to cloud point. The sample was slowly cooled down to rt. Many particles precipitated out. The solid was collected by vacuum filtration, dried under reduced pressure. The yield was 80.3%. The resulting solid was analyzed by XRPD according to the procedure in Compound II Crystallization Example 1 and the spectrum is shown as line B in FIG. 24.

### Compound II Crystallization

### Example 3

303.5 mg of Compound 4-3 (free base form of Compound II) was weighed into a vial and dissolved by 1 mL of MeOH at 60° C. with stirring. 153 μL of 5 M HCl (in EtOH) was added. Into the vial, 10 mL of acetone was slowly added. The sample was slowly cooled down to rt at a rate of 3° C./h. The solid was collected by vacuum filtration, dried under reduced pressure overnight. The yield was 69.5%. The resulting solid was analyzed by XRPD according to the procedure in Compound II Crystallization Example 1 and the spectrum is shown as line C in FIG. 24.

### Compound II Crystallization

### Example 4

311.2 mg of Compound 4-3 (free base form of Compound II) was weighed into a vial and dissolved by addition of 1 mL of MeOH at 60° C. with stirring. 157 μL of 5 M HCl (in EtOH) was added. Into the vial, 10 mL of acetone was slowly added. The sample was slowly cooled down to rt at a rate of 3° C./h. The solid was collected by vacuum filtration, dried under reduced pressure overnight. The yield was 59.4%. The resulting solid was analyzed by XRPD according to the procedure in Compound II Crystallization Example 1 and the spectrum is shown as line D in FIG. 24.

### Compound II Crystallization

### Example 5

333.5 mg of Compound II was weighed into a vial and dissolved by addition of 1 mL of MeOH at 55° C. with stirring. 168 μL of 5 M HCl (in EtOH) was added. Into the vial, 8 mL of acetone and 0.5 mL of MTBE were slowly added. The sample was slowly cooled down to rt at a rate of 3° C./h. A gel formed. The sample was dried under a stream of nitrogen.

To the vial, 1 mL of MeOH was added at 50° C. with stirring. A clear solution was formed. 10 mL of acetone was added with stirring to cloud point. The sample was slowly cooled down to rt. Many particles precipitated out. The solid was collected by vacuum filtration, dried under reduced pressure overnight. The yield was 73.9%. The resulting solid was analyzed by XRPD according to the procedure in Compound II Crystallization Example 1 and the spectrum is shown as line E in FIG. 24.

### Compound II Crystallization

### Example 6

121.2 mg of Compound 4-3 (free base form of Compound II) was weighed into a vial and dissolved by 1 mL of IPA. 51 μL of 6 M HCl was added with stirring at 65° C. A clear solution formed. 3.6 mL of acetone was added to cloud point with stirring. The sample was slowly cooled down to rt at a 3° C./h. No significant change was observed. The sample was dried under a stream of nitrogen.

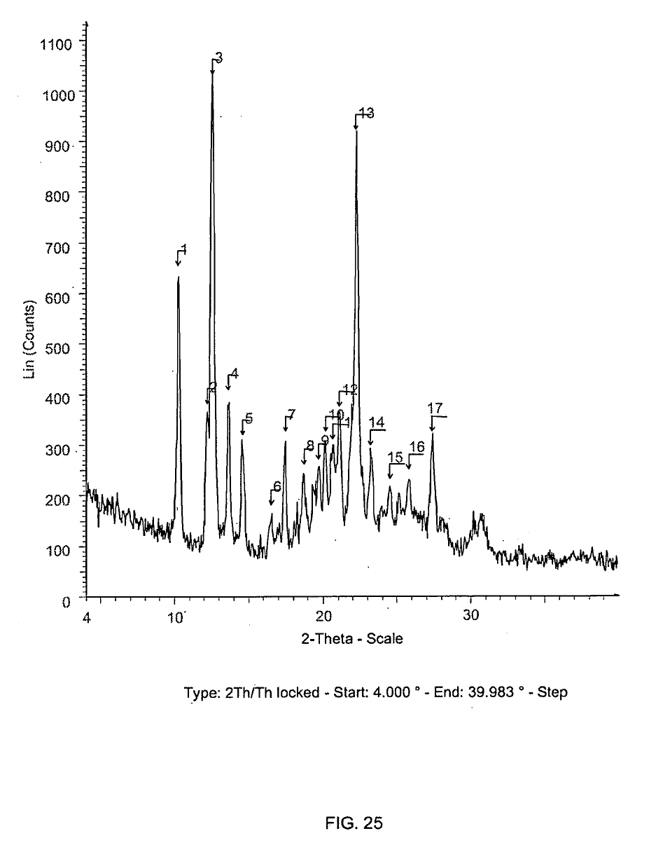

Into the vial, 0.5 mL EtOH was added at 60° C. with stirring. A clear solution formed. 4 mL of acetone was added to cloud point. The sample was slowly cooled down to rt at a rate of 3° C./h. No significant change was observed. The sample was dried under a stream of Nitrogen. Into the vial, 1 mL MeOH was added at 65° C. A clear solution formed, 8 mL of acetone, 1.0 mL MTBE were added to cloud point with stirring. The sample was slowly cooled down to rt. No significant change was observed. Into the vial, 1 mL MeOH was added at 60° C. A clear solution formed. 8 mL of acetone was added as anti-solvent. The sample was slowly cooled down to rt at a rate of 3° C./h. No significant change was observed. 1.2 mL MTBE was added to cloud point while the system was warmed up back to 60° C. with stirring. The sample was slowly cooled down to rt at a rate of 3° C./h. Many particles precipitated out. The solid was collected by vacuum filtration, dried under reduced pressure for 3 days. The yield was 78.7%. The resulting solid was analyzed by XRPD according to the procedure in Compound II Crystallization Example 1 and the spectrum is shown as line F in FIG. 24. Additionally, the spectrum for this sample is shown in greater detail at FIG. 25. The data for the numbered peaks in FIG. 25 is shown in Table 8.

### Compound II Crystallization

### Example 7

101.0 mg of Compound 4-3 (free base form of Compound II) was weighed into a vial and dissolved by 1 mL of ethanoUIPA (11/4 (v/v)). 42.5 μL of 6 M HCl was added with stirring at 50° C. Then the solution was evaporated under a stream of nitrogen. Gel like solid formed.

To the vial, 2 mL of EtOH/IPA (11/4 (v/v)) was added at 50° C. with stirring. A clear solution formed. 5 mL of MTBE was added with stirring, resulting in a little precipitates on contact. The sample was slowly cooled down to rt. Many particles precipitated out. The solid was collected by vacuum filtration, dried under reduced pressure for 2 days. The yield was 46.2%. The resulting solid was analyzed by XRPD according to the procedure in Compound II Crystallization Example 1 and the spectrum is shown as line G in FIG. 24.

### Compound II Crystallization

### Example 8

100.9 mg of Compound 4-3 was weighed into a vial and dissolved by 1.0 mL of EtOH at 65° C. with stirring. 43 μL of 6 M HCl was added with stirring at 60° C. 2 mL of MTBE was added to cloud point. The sample was slowly cooled down to room temperature. A gel formed. The sample was dried under a stream of nitrogen.

Into the vial, 2.0 mL of EtOH was added at 65° C. with stirring. A clear solution formed. 2.5 mL of MTBE was added to cloud point. The sample was slowly cooled down to room temperature. A gel formed. The sample was dried under a stream of nitrogen.

Into the vial, 2.0 mL of MeOH was added at 65° C. with stirring. A clear solution formed. 3.0 mL of di-isopropyl ether was added to cloud point. The sample was slowly cooled down to room temperature. A gel formed.

Into the vial, 1.0 mL of 88% acetone was added at 60° C. with stirring. A clear solution formed. 2.5 mL of ACN was added to cloud point. The sample was slowly cooled down to room temperature. A gel formed.

Into the vial, 1.0 mL of MeOH was added at 60° C. with stirring. A clear solution formed. 8.0 mL of acetone was added. The sample was slowly cooled down (3° C./h) to room temperature. A lot of fine crystalline formed which turned out to be very hygroscopic under the polarized microscope. The solid was collected by vacuum filtration and dried in a vacuum oven over the weekend at 45° C., resulting in 57.6% recovery.

The solid was analyzed by XRPD according to the procedure in Compound II Crystallization Example 1 and the spectrum is shown as line H in FIG. 24.

This sample was analyzed microscopically. Microscopy was performed using a Leica DMLP polarized light microscope equipped with 2.5×, 10× and 20× objectives and a digital camera to capture images showing particle shape, size, and crystallinity. Crossed polarizers were used to show birefringence and crystal habit for the samples dispersed in immersion oil. The sample had an irregular crystal habit as shown in FIG. 26.

This sample was analyzed thermogravimetrically. Thermogravimetric analyses were carried out on a TA Instrument TGA unit (Model TGA 500). Samples were heated in platinum pans from 25 to 300° C. at 10° C./min with a nitrogen purge of 50 mL/min. The TGA temperature was calibrated with nickel standard, MP=354.4° C. The weight calibration was performed with manufacturer-supplied standards and verified against sodium citrate dihydrate desolvation. The resulting thermogram is shown in FIG. 27. The sample shows a weight percentage loss of 1.751% from 25.0-120° C. and 3.485% from 25.0-210° C.

The sample was analyzed calorimetrically. Differential scanning calorimetry analyses were carried out on a TA Instrument DSC unit (Model DSC 1000). Samples were heated in non-hermetic aluminum pans from 25 to 300° C. at 10° C./min with a nitrogen purge of 50 mL/min. The DSC temperature was calibrated with indium standard, onset of 156-158° C., enthalpy of 25-29 J/g. As shown in FIG. 28, the sample had an endothermic onset at 37.63° C. due to loss of volatiles, followed by a melting decomposition at 246.54° C.

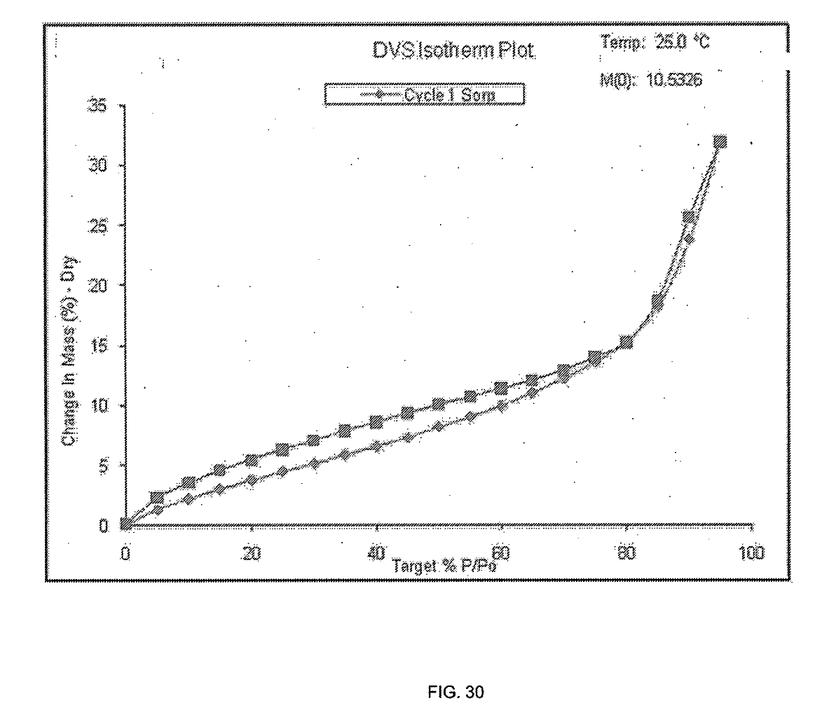

The moisture sorption profile was generated of the sample as well as of a sample of amorphous Compound II at 25° C. using a DVS Moisture Balance Flow System (Model Advantage) with the following conditions: sample size approximately 10 mg, drying 25° C. for 60 minutes, adsorption range 0% to 95% RH, desorption range 95% to 0% RH, and step interval 5%. The equilibrium criterion was <0.01% weight change in 5 minutes for a maximum of 120 minutes. As shown in FIG. 29, the sample was medium hygroscopic with 4.34% weight percentage change from 0-75% RH. It absorbed water very quickly at ˜85% RH and above. The amorphous Compound II, by contrast, would take up 13.57% of water from 0-75% RH as shown in FIG. 30.

### Compound II Crystallization

### Example 9

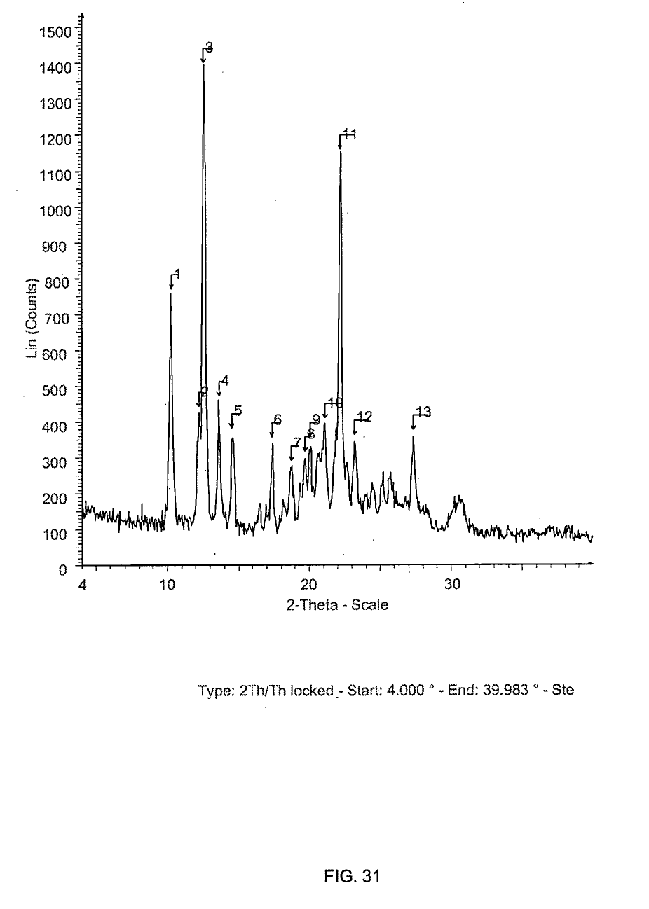

Compound 4-3 (free-base, 5.0 g) was dissolved in 15.0 mL of MeOH at 65° C. with stirring. HCl in EtOH (5 M, 3.75 mL) was added, and the resulting solution was stirred at 65° C. for 15 min, still a clear solution. Acetone (150 mL) was added dropwise over a period of 1.5 h until the cloud point was reached. The sample was kept stirring at 65° C. for 1 hr, and then gradually cooled down (˜10° C./h) to rt (30° C.). The mixture was stirred at this temperature overnight. The solid was collected by filtration, washed with acetone (5 mL×3) and dried in vacuum to give 4.4 g of product as a pale yellow solid, the yield was 80.4%. The resulting solid was analyzed by XRPD as in Compound II Crystallization Example 1. The spectrum is shown at FIG. 31 and numbered peaks identified in Table 9 in Appendix A.

### Solubility of Form I

Solubility of Form I as well as the free base compound 4-3 was tested. Solubility was measured by placing a small quantity of the compound to be analyzed in a glass vial, capping and rotating the vial overnight at ambient conditions (24 hours). Target concentration was 2.0 mg/mL. The samples were filtrated with 0.45-μm filters. The subsequent filtrate was collected for HPLC assay. HPLC conditions are shown in Table 10 in Appendix A. Solubility for Form I is shown in Table 11 and for the free base compound 4-3 is shown in Table 12 in Appendix A.

### Biological Activity Example

The ability of the disclosed compounds to inhibit HCV replication can be demonstrated in in vitro assays. Biological activity of the compounds of the invention was determined using an HCV replicon assay. The 1b_Huh-Luc/Neo-ET cell line persistently expressing a bi-cistronic genotype 1b replicon in Huh 7 cells was obtained from ReBLikon GMBH. This cell line was used to test compound inhibition using luciferase enzyme activity readout as a measurement of compound inhibition of replicon levels.

On Day 1 (the day after plating), each compound was added in triplicate to the cells. Plates incubated for 72 hrs prior to running the luciferase assay. Enzyme activity was measured using a Bright-Glo Kit (cat. number E2620) manufactured by Promega Corporation. The following equation was used to generate a percent control value for each compound.

% Control=(Average Compound Value/Average Control)*100

The EC₅₀ value was determined using GraphPad Prism and the following equation:

Y=Bottom+(Top−Bottom)/(1+10̂((LogIC50−X)*HillSlope))

EC₅₀ values of compounds are repeated several times in the replicon assay.

The disclosed compounds can inhibit multiple genotypes of HCV including, but not limited to 1a, 1b, 2a, 3a, 4a and 5a. The EC₅₀s are measured in the corresponding replicon assays that are similar to HCV 1b replicon assay as described above.

| | Average EC₅₀ (nM, n > 3) | | | | | |
|---|---|---|---|---|---|---|
| Genotypes | GT 1a | GT 1b | GT 2a | GT 3a | GT 4a | GT 5a |
| Compound I | 0.135 | 0.016 | 0.139 | 1.256 | 0.052 | 0.039 |
| Compound II | 0.07 | 0.01 | 0.11 | 0.40 | 0.04 | 0.04 |

### Pharmacokinetic Studies and Data of Compound I and Compound II in Preclinical Species.

The pharmacokinetics (PK) properties of Form A of Compound I and Form I of Compound II were determined in a series of comprehensive experiments in preclinical species including Sprague-Dawley rats, beagle dogs, cynomolgus monkeys.

In those studies, the Form A crystalline salt of Compound I (and Form I crystalline salt of Compound II) was formulated in saline, 0.5% MC in saline or other commonly used suitable formulation vehicles to give a clear solution or as a suspension or a paste depending on the concentration intended to reach and the choice of vehicles. Dosing was by oral gavage. Blood samples were drawn and placed into individual tube containing K₂EDTA. Blood samples were put on ice and centrifuged (2000 g for 5 minutes at 4° C.) to obtain plasma within 15 minutes after collection. Plasma samples were stored at approximately −80° C. freezer until analysis.

For most of these analyses, Compound I (and Compound II) and the internal standard (IS) were extracted from rat, monkey or dog plasma by protein precipitation, and the extract was evaporated, reconstituted and analyzed using HPLC with tandem mass spectrometric detection (HPLC-MS/MS), see examples for further details. Calibration was accomplished by weighted linear regression of the ratio of the peak area of analyte to that of the added internal standard (IS). For the validated assay in rat and monkey EDTA plasma, the Lower Limit of Quantitation (LLOQ) for both compound 1 and 2 was 5.00 ng/mL, and the assay was linear from 5.00-1,000 ng/mL. PK parameters were calculated by non-compartmental analysis using WinNonlin (versions 4.1 through 6.1).

### Example 1

### A PK Study of Compound I in Rats

Dosing Formulation Preparation:

1) Weighed 922.80 mg of Form A of Compound I (equivalent to 824.603 mg of free base) into a clean tube. 2) Added 54.974 mL of 0.5% methylcellulose in saline into the tube containing the Form A of Compound I, vortexed for 3-5 min and sonicated for 10-15 min. The dosing solution was a light yellow and clear solution.

Sprague Dawley rats, ˜7-9 weeks old and weighing ˜210-270 g, were given the above dosing solution at 5 mL/kg. Blood samples were collected into individual tubes containing K₂EDTA at time points of pre-dose, 0.083, 0.25, 0.5, 1, 2, 4, 6, 8, 24 hr post dose.

Sample Preparation for Analysis: An aliquot of 30 μL of plasma sample was mixed with 30 μL of the IS (200 ng/mL), then mixed with 150 μL ACN for protein precipitation. The mixture was vortexed for 2 min and centrifuged at 12000 rpm for 5 min. An aliquot of 1 μL of supernatant was injected onto HPLC-MS/MS, if no further dilution was needed. To prepare a 10-fold diluted plasma samples, an aliquot of 10 μL plasma sample was mixed with 90 μL blank plasma to obtain the diluted plasma samples. The extraction procedure for diluted samples was as the same as that used for the non-diluted samples.
Compound Concentration Quantitation:
Instrument HPLC-MS/MS-12 (API4000), on positive ionization mode, ESI+
HPLC Conditions Mobile Phase A: H₂O—0.025% formic acid (FA)—1 mM NH₄OAc
   Mobile Phase B: MeOH—0.025% FA—1 mM NH₄OAc

| Time (min) | Pump B (%) |
|---|---|
| 0.20 | 10 |
| 0.60 | 95 |
| 1.30 | 95 |
| 1.35 | 10 |
| 1.50 | Stop |

Column: ACQUITY UPLC BEH C18 (2.1×50 mm, 1.7 μm)
Oven temperature: 60° C.
Flow rate: 0.80 mL/min

Pharmacokinetic analysis was done using the WinNonlin software (Version 5.3, Pharsight Corporation, California, USA). Non-compartmental model pharmacokinetic parameters were estimated and presented in the tables. Any concentration data under LLOQ (LLOQ=1.00 ng/mL in rat plasma and 3.00 ng/mL in rat liver homogenate) were replaced with “BQL”.

TABLE 13
Individual and mean plasma concentration (ng/mL)-time
data of Form A of Compound I after a single PO
dose of 75 mg/kg in male SD rats (N = 3)

| Time (hr) | Rat #1 | Rat #2 | Rat #3 | Mean | SD | CV (%) |
|---|---|---|---|---|---|---|
| 1 | 6210 | 5480 | 6230 | 5973 | 427 | 7.15 |
| 2 | 8030 | 7890 | 6380 | 7433 | 915 | 12.3 |
| 3 | 5670 | 8060 | 11300 | 8343 | 2826 | 33.9 |
| 4 | 3680 | 5170 | 2930 | 3927 | 1140 | 29.0 |
| 6 | 2780 | 3370 | 2400 | 2850 | 489 | 17.1 |
| 8 | 498 | 704 | 390 | 531 | 160 | 30.1 |
| 12 | 188 | 277 | 63.6 | 176 | 107 | 60.8 |
| 24 | 8.12 | 7.95 | 8.15 | 8.07 | 0.108 | 1.34 |

### Example 2

### A PK Study of Form A of Compound I in Dogs

Non-naïve Beagle Dog, 8.0-9.5 kg were used in the study. The dosing solution was prepared by dissolving 1.90 g of Form A of Compound I (1.67 g free base equivalent) in 222.237 mL of 0.5% MC and vortexed for 20 min, sonicated for 2 min to obtain a colorless clear solution. The animals were restrained manually, and approx. 0.6-1 mL blood/time point was collected from cephalic or saphenous veins into pre-cooled EDTA tubes. Blood samples were put on ice and centrifuged at 4° C. to obtain plasma within 30 minutes of sample collection. Plasma samples were stored at approximately −70° C. until analysis.

### Quantitation by LC-MS/MS

Instrument LC-MS/MS-010 (API4000)
Internal standard(s) Testosterone
HPLC conditions Mobile Phase A: H₂O—5 mM NH₄OAc
   Mobile Phase B: MeOH—5 mM NH₄OAc

| Time (min) | Pump B (%) |
|---|---|
| 0.30 | 10 |
| 0.90 | 95 |
| 2.0 | 95 |
| 2.1 | 10 |
| 3.50 | stop |

Column: Boston ODS (2.1×50 mm, 5 μm)
Guard column: Security Guard C18 (4.0×2.0 mm, 5 μm)
Flow rate: 0.40 mL/min
Retention time
Compound I retention time: 2.44 min
IS retention time: 2.46 min
Sample preparation For plasma samples:
   An aliquot of 30 μL plasma was added with 200 μL IS in ACN (testosterone as IS, 100.0 ng/mL), the mixture was vortexed for 2 min and centrifuged at 12000 rpm for 5 min. 5 μL of the supernatant was injected for LC-MS/MS analysis.
   For dilution samples:
   An aliquot of 10 μL plasma sample was added with 90 μL blank Beagle dog plasma. The dilution factor was 10. An aliquot of 30 μL dilution plasma was added with 200 μL IS in ACN (testosterone as IS, 100.0 ng/mL), the mixture was vortexed for 2 min and centrifuged at 12000 rpm for 5 min. 5 μL of the supernatant was injected for LC-MS/MS analysis.

TABLE 14
Individual and mean plasma concentration (ng/mL)-time data
of Form A of Compound I after a PO dose of 75 mg/kg in
Beagle Dog

| | | | Mean |
|---|---|---|---|
| Time (hr) | Dog #1 | Dog #2 | (ng/mL) |
| Predose | BQL | BQL | BQL |
| 0.083 | BQL | 5.92 | 5.92 |
| 0.25 | 267 | 374 | 320 |
| 0.5 | 1091 | 952 | 1021 |
| 1 | 1589 | 2051 | 1820 |
| 2 | 2979 | 2404 | 2692 |
| 4 | 3079 | 1536 | 2307 |
| 6 | 3587 | 1320 | 2454 |
| 8 | 3503 | 1043 | 2273 |
| 24 | 244 | 2161 | 1203 |

### Example 3

### PK Study of Form I of Compound II in Monkeys

Non-naïve Cynomolgus monkeys, 3.2-3.5 kg, male

Dosing solution was prepared by dissolving 682.96 mg of Form I of Compound II in 82.558 mL of 0.5% MC in saline, vortexing for 5 min and sonicating for 18 min to obtain a homogenous solution. The above solution was given to the animals at 10 mL/kg via intragastric administration

To collect blood samples, the animals were restrained manually and approx. 0.6-1 mL blood/time point was collected from cephalic or sephanous veins into pre-cooled EDTA tubes. Blood samples were put on ice and centrifuged at 4° C. to obtain plasma within 30 minutes of sample collection. Plasma samples were stored at −70° C. until analysis.
Instrument LC-MS/MS (API4000)
MS conditions Positive ion, EST
HPLC conditions Mobile Phase A: H₂O—0.025% formic acid—1 mM NH₄OAc
   Mobile Phase B: MeOH—0.025% formic acid—1 mM NH₄OAc

| Time (min) | Pump B (%) |
|---|---|
| 0.30 | 10 |
| 0.90 | 95 |
| 2.00 | 95 |
| 2.10 | 10 |
| 3.50 | Stop |

Column: Boston Crest ODS-C18 (2.1×50 mm, 5 μm)
Flow rate: 0.40 mL/min
Compound II retention time: 2.30 min;
IS retention time: 2.29 min
Sample preparation An aliquot of 20 μL plasma sample was protein precipitated with 3004 ACN which contains 5 ng/mL IS (P1100970-1). The mixture was vortexed for 2 min, and then centrifuged at 12000 rpm for 5 min. an aliquot of 5 μL supernatant was injected onto the LC-MS/MS system.

TABLE 15
Individual and mean plasma concentration (ng/mL)-time data
of Form I of Compound II after a PO dose of 75 mg/kg in
Cynomolgus monkeys

| Time (hr) | #0612169 | #0610703 | Mean |
|---|---|---|---|
| Predose | BQL | BQL | BQL |
| 0.083 | 8.33 | BQL | 8.33 |
| 0.25 | 86.8 | 33.3 | 60.1 |
| 0.5 | 273 | 207 | 240 |
| 1 | 1140 | 497 | 819 |
| 2 | 954 | 336 | 645 |
| 4 | 435 | 213 | 324 |
| 6 | 194 | 110 | 152 |
| 8 | 93.1 | 55.9 | 74.5 |
| 24 | 7.37 | 8.79 | 8.08 |

### (c) Pharmaceutical Compositions

Certain embodiments provided herein are pharmaceutical compositions comprising the solid forms described herein. In a first embodiment, the pharmaceutical composition further comprises one or more pharmaceutically acceptable excipients or vehicles, and optionally other therapeutic and/or prophylactic ingredients. Such excipients are known to those skilled in the art.

Depending on the intended mode of administration, the pharmaceutical compositions may be in the form of solid or semi-solid dosage forms, such as, for example, tablets, suppositories, pills, capsules, powders, suspensions, creams, ointments, lotions or the like, and in some embodiments, in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a solid form described herein together with one or more pharmaceutically acceptable carriers and optionally other therapeutic and/or prophylactic ingredients.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate and the like.

For oral administration, the composition will generally take the form of a tablet, capsule, or suspension. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents and the like.

In some embodiments, provided herein are dosage forms consisting of the solid form alone, i.e., a solid form without any excipients. In some embodiments, provided herein are sterile dosage forms comprising the solid forms described herein.

In one embodiment Compound I is administered without any excipients in size zero Swedish Orange opaque hydroxypropylmethylcellulose (HPMC) capsules. Approximately 44 mg of Compound I powder is filled into each HPMC capsule.

Certain embodiments herein provide the use of the solid forms described herein in the manufacture of a medicament. In further embodiments, the medicament is for the treatment of hepatitis C.

### (d) Methods of Use

Certain embodiments herein provide a method of treating hepatitis C comprising administering to a subject in need thereof, a therapeutically effective amount of a solid form described herein, optionally in a pharmaceutical composition. A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. The subject may be administered as many doses as is required to reduce and/or alleviate the signs, symptoms or causes of the disorder in question, or bring about any other desired alteration of a biological system. One of ordinary skill in the art of treating such diseases will be able, without undue experimentation and in reliance upon personal knowledge and the disclosure of this application, to ascertain a therapeutically effective amount of the compounds of this invention for a given disease.

### (e) Combination Therapy

The solid forms and pharmaceutical compositions described herein are useful in treating and preventing HCV infection alone or when used in combination with other compounds targeting viral or cellular elements or functions involved in the HCV lifecycle. Classes of compounds useful in the invention may include, without limitation, all classes of HCV antivirals. For combination therapies, mechanistic classes of agents that may be useful when combined, including for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes include, but are not limited to, macrocyclic, heterocyclic and linear HCV protease inhibitors such as Telaprevir (VX-950), Boceprevir (SCH-503034), Narlaprevir (SCH-900518), ITMN-191 (R-7227), TMC-435350 (a.k.a. TMC-435), MK-7009, BI-201335, BI-2061 (Ciluprevir), BMS-650032 (Asunaprevir), ACH-1625, ACH-1095 (HCV NS4A protease co-factor inhibitor), VX-500, VX-813, PHX-1766, PHX2054, IDX-136, IDX-316, ABT-450, EP-013420 (and congeners) and VBY-376; the Nucleosidic HCV polymerase (replicase) inhibitors useful in the invention include, but are not limited to, R7128, PSI-7851, IDX-184, IDX-102, R1479, UNX-08189, PSI-6130, PSI-938, PSI-879 and PSI-7977 (GS-7977, Sofosbuvir) and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nuclosidic HCV polymerase (replicase) inhibitors useful in the invention, include, but are not limited to, PPI-383, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, solid forms and compositions described herein may be used in combination with cyclophyllin and immunophyllin antagonists (e.g., without limitation, DEBIO compounds, NM-811 as well as cyclosporine and its derivatives), kinase inhibitors, inhibitors of heat shock proteins (e.g., HSP90 and HSP70), other immunomodulatory agents that may include, without limitation, interferons (-alpha, -beta, -omega, -gamma, -lambda or synthetic) such as intron A™, Roferon-A™, Canferon-A300™, Advaferon™, Infergen™, Humoferon™, Sumiferon MP™, Alfaferone™, IFN-β™, Feron™ and the like; polyethylene glycol derivatized (pegylated) interferon compounds, such as PEG interferon-α-2a (Pegasys™), PEG interferon-α-2b (PEGIntron™), pegylated IFN-α-con1 and the like; long acting formulations and derivatizations of interferon compounds such as the albumin-fused interferon, Albuferon™, Locteron™ and the like; interferons with various types of controlled delivery systems (e.g. ITCA-638, omega-interferon delivered by the DUROS™ subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effectiveamount of a TNF-α antagonist. Exemplary, non-limiting TNF-α antagonists that are suitable for use in such combination therapies include ENBREL™, REMICADE™ and HUMIRA™.

In addition, solid forms and compositions described herein may be used in combination with antiprotozoans and other antivirals thought to be effective in the treatment of HCV infection, such as, without limitation, the prodrug nitazoxanide. Nitazoxanide can be used as an agent in combination the compounds disclosed in this invention as well as in combination with other agents useful in treating HCV infection such as peginterferon alfa-2a and ribavarin

(see, for example, Rossignol, J F and Keeffe, E B, Future Microbiol. 3:539-545, 2008).

The solid forms and compositions described herein may also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. No. 5,807,876; U.S. Pat. No. 6,498,178; U.S. Pat. No. 6,344,465; U.S. Pat. No. 6,054,472; WO97/40028; WO98/40381; WO00/56331, WO 02/04425; WO 03/007945; WO 03/010141; WO 03/000254; WO 01/32153; WO 00/06529; WO 00/18231; WO 00/10573; WO 00/13708; WO 01/85172; WO 03/037893; WO 03/037894; WO 03/037895; WO 02/100851; WO 02/100846; EP 1256628; WO 99/01582; WO 00/09543; WO02/18369; WO98/17679, WO00/056331; WO 98/22496; WO 99/07734; WO 05/073216, WO 05/073195 and WO 08/021927, the entireties of which are incorporated herein by reference.

Additionally, combinations of, for example, ribavirin and interferon, may be administered as multiple combination therapy with at least one of solid forms or compositions described herein. Combinable agents are not limited to the aforementioned classes or compounds and contemplates known and new compounds and combinations of biologically active agents (see, Strader, D. B., Wright, T., Thomas, D. L. and Seeff, L. B., AASLD Practice Guidelines. 1-22, 2009 and Maims, M. P., Foster, G. R., Rockstroh, J. K., Zeuzem, S., Zoulim, F. and Houghton, M., Nature Reviews Drug Discovery. 6:991-1000, 2007, Pawlotsky, J-M., Chevaliez, S. and McHutchinson, J. G., Gastroenterology. 132:179-1998, 2007, Lindenbach, B. D. and Rice, C. M., Nature 436:933-938, 2005, Klebl, B. M., Kurtenbach, A., Salassidis, K., Daub, H. and Herget, T., Antiviral Chemistry & Chemotherapy. 16:69-90, 2005, Beaulieu, P. L., Current Opinion in Investigational Drugs. 8:614-634, 2007, Kim, S-J., Kim, J-H., Kim, Y-G., Lim, H-S. and Oh, W-J., The Journal of Biological Chemistry. 48:50031-50041, 2004, Okamoto, T., Nishimura, Y., Ichimura, T., Suzuki, K., Miyamura, T., Suzuki, T., Moriishi, K. and Matsuura, Y., The EMBO Journal. 1-11, 2006, Soriano, V., Peters, M. G. and Zeuzem, S. Clinical Infectious Diseases. 48:313-320, 2009, Huang, Z., Murray, M. G. and Secrist, J. A., Antiviral Research. 71:351-362, 2006 and Neyts, J., Antiviral Research. 71:363-371, 2006, each of which is incorporated by reference in their entirety herein). It is intended that combination therapies described herein include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy.

### APPENDIX A

TABLE 1
Observed peaks for Compound (I) diHCl.

| | | Intensity |
|---|---|---|
| °2θ | d space (Å) | (%) |
| 10.18 ± 0.20 | 8.692 ± 0.174 | 19 |
| 10.59 ± 0.20 | 8.350 ± 0.160 | 100 |
| 12.32 ± 0.20 | 7.187 ± 0.118 | 33 |
| 12.67 ± 0.20 | 6.988 ± 0.112 | 93 |
| 13.59 ± 0.20 | 6.518 ± 0.097 | 30 |
| 14.09 ± 0.20 | 6.287 ± 0.090 | 12 |
| 14.69 ± 0.20 | 6.031 ± 0.083 | 31 |
| 16.26 ± 0.20 | 5.451 ± 0.067 | 13 |
| 17.08 ± 0.20 | 5.192 ± 0.061 | 12 |
| 17.41 ± 0.20 | 5.093 ± 0.059 | 41 |
| 18.15 ± 0.20 | 4.888 ± 0.054 | 10 |
| 18.47 ± 0.20 | 4.805 ± 0.052 | 11 |
| 18.72 ± 0.20 | 4.741 ± 0.051 | 12 |
| 19.18 ± 0.20 | 4.626 ± 0.048 | 14 |
| 20.15 ± 0.20 | 4.406 ± 0.044 | 20 |
| 20.44 ± 0.20 | 4.346 ± 0.042 | 34 |
| 21.06 ± 0.20 | 4.219 ± 0.040 | 33 |
| 21.49 ± 0.20 | 4.135 ± 0.038 | 18 |
| 22.06 ± 0.20 | 4.030 ± 0.036 | 100 |
| 22.41 ± 0.20 | 3.967 ± 0.035 | 23 |
| 22.76 ± 0.20 | 3.907 ± 0.034 | 15 |
| 23.41 ± 0.20 | 3.800 ± 0.032 | 15 |
| 24.48 ± 0.20 | 3.636 ± 0.029 | 18 |
| 25.58 ± 0.20 | 3.482 ± 0.027 | 15 |
| 26.02 ± 0.20 | 3.425 ± 0.026 | 11 |
| 27.04 ± 0.20 | 3.298 ± 0.024 | 25 |
| 27.47 ± 0.20 | 3.247 ± 0.023 | 11 |
| 28.34 ± 0.20 | 3.149 ± 0.022 | 9 |
| 29.28 ± 0.20 | 3.050 ± 0.021 | 8 |

TABLE 2
Prominent peaks for Compound (I) diHCl.

| °2θ | d space (Å) | Intensity (%) |
|---|---|---|
| 10.59 ± 0.20 | 8.350 ± 0.160 | 100 |
| 12.67 ± 0.20 | 6.988 ± 0.112 | 93 |
| 13.59 ± 0.20 | 6.518 ± 0.097 | 30 |
| 14.69 ± 0.20 | 6.031 ± 0.083 | 31 |
| 17.41 ± 0.20 | 5.093 ± 0.059 | 41 |

TABLE 3

| | 448014.Fsh |
|---|---|
| Experiment | Temp-RH |
| Operator | DSO |
| Experiment ID | 448014 |
| Sample Name | |
| Sample Lot # | 4252-52-01, LIMS #258446 |
| Notes | Range 5% to 95% |
| | 25° C. at 10% increments |
| Drying | OFF |
| Max Equil Time | 180 min |
| Equil Crit | 0.0100 wt % in 5.00 min |
| T-RH Steps | 25, 5; 25, 15; 25, 25; 25, 35; 25, 45; 25, 55; 25, 65; |
| | 25, 75; 25, 85; 25, 95; 25, 85; 23, 75; 25, 65; 25, 55; |
| | 25, 45; 25, 35; 25, 25; 25, 15; 25. 5 |
| Data Logging Interval | 2.00 min or 0.0100 wt % |
| Expt Started | Mar. 6, 2011 |
| Run Started | 11:20:36 |

| Step Time | Elap Time | Weight | Weight | Samp Temp | Samp RH |
|---|---|---|---|---|---|
| min | min | mg | % chg | deg C. | % |
| n/a | 0.1 | 12.351 | 0.000 | 25.15 | 1.33 |
| 52.8 | 52.9 | 12.328 | −0.187 | 25.17 | 5.09 |
| 30.7 | 83.6 | 12.359 | 0.065 | 25.17 | 14.73 |
| 28.1 | 111.6 | 12.388 | 0.303 | 25.16 | 24.93 |
| 24.8 | 136.5 | 12.415 | 0.521 | 25,16 | 34.87 |
| 40.6 | 177.1 | 12.447 | 0.780 | 25.16 | 44.91 |
| 37.4 | 214.5 | 12.484 | 1.082 | 25.16 | 54.80 |
| 55.4 | 269.9 | 12.531 | 1.458 | 25.16 | 64.95 |
| 50.2 | 320.1 | 12.581 | 1.868 | 25.15 | 74.71 |
| 73.9 | 393.9 | 12.660 | 2.502 | 25.16 | 84.57 |
| 187.8 | 581.7 | 13.687 | 10.824 | 25.16 | 94.86 |
| 117.5 | 699.2 | 13.321 | 7.855 | 25.16 | 85.24 |
| 79.8 | 779.0 | 13.183 | 6.741 | 25.16 | 75.27 |
| 97.8 | 876.8 | 13.091 | 5.993 | 25.16 | 65.08 |
| 76.6 | 953.4 | 13.012 | 5.352 | 25.16 | 55.17 |
| 124.1 | 1077.5 | 12.918 | 4.590 | 25.16 | 44.99 |
| 92.3 | 1169.8 | 12.829 | 3.870 | 25.17 | 35.13 |
| 105.0 | 1274.8 | 12.724 | 3.025 | 25.17 | 25.12 |
| 91.7 | 1366.5 | 12.599 | 2.010 | 25.17 | 14.73 |
| 70.7 | 1437.1 | 12.434 | 0.673 | 25.17 | 5.17 |

weight changes (the percentages are with respect to the initial sample
weight)

| | |
|---|---|
| −0.187 | % wt change upon equilibration at 5% RH |
| 2.689 | % wt gain from 5%-85% RH |
| 8.322 | % wt gain from 85%-95% RH |
| 10.151 | % wt lost from 95%-5% RH |

TABLE 4

| METILER TOLEDO DL39 V2.20 | | | Serial No. 5128049522 |
|---|---|---|---|
| KFC3/Dow | | | |
| Method: | 102 | Ext. Solv. | 3/21/2011 11:14 AM |
| Start time: | 3/21/2011 11:15 AM | | |

Sample data

| No. | Note/ID | Start time | Sample Size |
|---|---|---|---|
| 1 | 259816, 4419-13-01 | 3/21/2011 11:15 AM | 0.9818 g |

Result

| | | | |
|---|---|---|---|
| No. | Note/ID | Start time | Sample Size and results |

| | | | | | |
|---|---|---|---|---|---|
| 1 | 259616, 4419-13-01 | 3/21/2011 11:15 AM | | 0.9818 | g |
| | | | R1 = | 2.06 | % |
| | | | R2 = | 0.00 | g |
| | | | R3 = | 0.00 | g |

Series note
Statistics

| Rx | Name | n | Mean value | Unit | e | srel [%] |
|---|---|---|---|---|---|---|
| R1 | | 1 | 2.06 | % | | |
| R2 | | 1 | 0.00 | g | | |
| R3 | | 1 | 0.00 | g | | |

Raw data

| | | | |
|---|---|---|---|
| Sample | | | |
| No. | 1 | | |
| Identification | 259618, 4419-13-01 | | |
| Note | | | |
| Titration stand | Internal stand | | |
| Mass | m = 0.9818 g | | |
| Stirrer speed | 35% | | |
| Mix time | 10 s | | |
| Blank | BLANK = 0 μg | | |
| Drift | DRIFT = 0 μg/min | | |
| KF determination | | | |
| Consumption | EP | CEQ1 = 7244.362 mC | |
| | | Q1 = 676.78 μg water | |
| Duration | | TIME = 138 s | (1356[0.1 s]) |
| Termination condition | Rel. drill | | |
| Calculation | | | |
| Result | R1 = 2.09 % | | |
| Formula | R1 = R1(%) * (f2 + f3)/f3 − f1 * f2/f3 | | |
| Factor | f1 = 0.0003 | | |
| Calculation | | | |
| Result | R2 = 0.000 g | | |
| Factor | f2 = 1.0022 | | |
| Calculation | | | |
| Result | R3 = 0.00 g | | |
| Factor | f3 = 0.0343 | | |

TABLE 5

| METTLER TOLEDO DL39 V2.20 | | | Serial No 5128049522 |
|---|---|---|---|
| KFC3/Dow | | | |
| Method: | 102 | Ext. Solv. | 3/21/2011 11:18 AM |
| Start time: | 3/21/2011 11:18 AM | | |

Sample data

| No. | Note/ID | Start time | Sample Size |
|---|---|---|---|
| 1 | 259618, 4419-13-01 | 3/21/2011 11:18 AM | 0.9388 g |

Results

| | | | |
|---|---|---|---|
| No. | Note/ID | Start time | Sample Size and results |

| | | | | | |
|---|---|---|---|---|---|
| 1 | 259618, 4419-13-01 | 3/21/2011 11:18 AM | | 0.9388 | g |
| | | | R1 = | 1.71 | % |
| | | | R2 = | 0.00 | g |
| | | | R3 = | 0.00 | g |

Series note
Statistics

| Rx | Name | n | Mean value | Unit | e | srel [%] |
|---|---|---|---|---|---|---|
| R1 | | 1 | 1.71 | % | | |
| R2 | | 1 | 0.00 | g | | |
| R3 | | 1 | 0.00 | g | | |

Raw data

| | | | |
|---|---|---|---|
| Sample | | | |
| No. | 1 | | |
| Identification | 259618, 4419-13-01 | | |
| Note | | | |
| Titration stand | Internal stand | | |
| Mass | m = 0.9388 g | | |
| Stirrer speed | 35% | | |
| Mix time | 10 s | | |
| Blank | BLANK = 0 μg | | |
| Drift | DRIFT = 0 μg/min | | |
| KF determination | | | |
| Consumption | EP | CEQ1 = 0493.739 mC | |
| | | Q1 = 686.61 μg water | |
| Duration | | TIME = 123 s | (1234[0.1 s]) |
| Termination condition | Rel drift | | |
| Calculation | | | |
| Result | R1 = 1.71 % | | |
| Formula | R1 = R1[%] * (f2 + f3)/f3 − f1 * f2/f3 | | |
| Factor | f1 = 0.0006 | | |
| Calculation | | | |
| Result | R2 = 0.00 g | | |
| Factor | f2 = 1.0403 | | |
| Calculation | | | |
| Result | R3 = 0.00 g | | |
| Factor | f3 = 0.0602 | | |

TABLE 6

| Angle | d value | Intensity | Intensity % |
|---|---|---|---|
| 2-Theta ° | Angstrom | Count | % |
| 10.251 | 8.62207 | 5679 | 17.5 |
| 10.677 | 8.27946 | 22190 | 68.5 |
| 12.389 | 7.13865 | 10907 | 33.6 |
| 12.778 | 6.92249 | 32413 | 100 |
| 13.679 | 6.46809 | 14147 | 43.6 |
| 14.763 | 5.99568 | 9432 | 29.1 |
| 16.308 | 5.43098 | 4690 | 14.5 |
| 17.49 | 5.06653 | 9393 | 29 |
| 19.341 | 4.58559 | 4811 | 14.8 |
| 20.516 | 4.32559 | 10629 | 32.8 |
| 21.13 | 4.20127 | 10305 | 31.8 |
| 22.143 | 4.01121 | 22923 | 70.7 |
| 22.79 | 3.89892 | 6354 | 19.6 |
| 23.491 | 3.78399 | 7151 | 22.1 |
| 24.562 | 3.62142 | 6842 | 21.1 |
| 25.662 | 3.46859 | 5930 | 18.3 |
| 26.03 | 3.42041 | 5139 | 15.9 |
| 27.113 | 3.28622 | 7384 | 22.8 |
| 27.666 | 3.23433 | 5077 | 15.7 |
| 28.345 | 3.14616 | 3847 | 11.9 |

TABLE 7

| Solvent | Sample No/ | | | Solubility |
|---|---|---|---|---|
| System^{a} | LIMS No | Tempertature^{b} | Conditions | (mg/mL)^{c} |
| IPA | 4362-98-03 | RT | aliquot addition | <3 |
| | | | slurry, 4 days | <3 |
| | 4362-98-05 | ~60° C. | aliquot addition | 2 |
| | 4362-88-01 | | slurry, 6 days | <6 |
| IPA:tBME | 4362-98-02 | RT | aliquot addition | <2 |
| (2:1) | | | slurry, 3 days | <2 |
| | 4362-98-04 | ~60° C. | aliquot addition | <3 |
| | | | slurry, 3 days | <3 |
| IPA:H₂O | 4362-98-01 | RT | aliquot addition | 33 |
| (95:5) | | | | |

^{a}Volume ratio given in parentheses for solvent mixtures.
^{b}RT = room temperature.
^{c}Solubilities are calculated basal on the total solvent used to give a solution; actual solubilities may be greater because of the volume of the solvent portions utilized or a slow rate of dissolution. Solubilities are reportod to the nearest mg/mL unless otherwise stated.

TABLE 8

| | | | | | Net | | |
|---|---|---|---|---|---|---|---|
| | | | | | Area | | |
| | Angle | d value | Intensity | Intensity | Cps × | | FWHM |
| # | °2θ | Å | Count | % | °2θ | Area % | °2θ |
| 1 | 10.218 | 8.64981 | 633 | 61.3 | 2.49 | 56.5 | 0.213 |
| 2 | 12.169 | 7.26745 | 363 | 35.1 | 0.258 | 5.9 | 0.146 |
| 3 | 12.507 | 7.07139 | 1033 | 100 | 3.571 | 81.0 | 0.24 |
| 4 | 13.588 | 6.51128 | 377 | 36.5 | 1.1 | 24.9 | 0.212 |
| 5 | 14.564 | 6.07733 | 309 | 29.9 | 0.923 | 20.9 | 0.238 |
| 6 | 16.493 | 5.3706 | 162 | 15.7 | 0.311 | 7.1 | 0.278 |
| 7 | 17.404 | 5.0915 | 306 | 29.6 | 0.792 | 18.0 | 0.217 |
| 8 | 18.683 | 4.74556 | 242 | 23.4 | 0.368 | 8.3 | 0.206 |
| 9 | 19.7 | 4.50294 | 255 | 24.7 | 0.171 | 3.9 | 0.17 |
| 10 | 20.115 | 4.41095 | 307 | 29.7 | 0.455 | 10.3 | 0.22 |
| 11 | 20.646 | 4.29863 | 298 | 28.8 | 0.335 | 7.6 | 0.283 |
| 12 | 21.083 | 4.21056 | 365 | 35.3 | 0.748 | 17.0 | 0.243 |
| 13 | 22.277 | 3.98751 | 922 | 89.3 | 4.41 | 100.0 | 0.253 |
| 14 | 23.239 | 3.82449 | 291 | 28.2 | 0.613 | 13.9 | 0.276 |
| 15 | 24.545 | 3.62394 | 216 | 20.9 | 0.212 | 4.8 | 0.221 |
| 16 | 25.831 | 3.44638 | 229 | 22.2 | 0.319 | 7.2 | 0.253 |
| 17 | 27.448 | 3.24691 | 321 | 31.1 | 1.073 | 24.3 | 0.276 |

TABLE 9

| | | | | | Net | | |
|---|---|---|---|---|---|---|---|
| | | | | | Area | | |
| | Angle | d value | Intensity | Intensity | Cps × | | FWHM |
| # | °2θ | Å | Count | % | °20 | Area % | °2θ |
| 1 | 10.195 | 8.66964 | 760 | 54.3 | 3.243 | 66.3 | 0.247 |
| 2 | 12.171 | 7.26601 | 424 | 30.3 | 0.41 | 8.4 | 0.187 |
| 3 | 12.539 | 7.05391 | 1399 | 100 | 4.895 | 100.0 | 0.221 |
| 4 | 13.573 | 6.51872 | 458 | 32.7 | 1.456 | 29.7 | 0.238 |
| 5 | 14.573 | 6.07349 | 353 | 25.2 | 1.211 | 24.7 | 0.246 |
| 6 | 17.331 | 5.11264 | 336 | 24 | 0.684 | 14.0 | 0.157 |
| 7 | 18.709 | 4.73896 | 273 | 19.5 | 0.72 | 14.7 | 0.255 |
| 8 | 19.689 | 4.50534 | 296 | 21.2 | 0.24 | 4.9 | 0.142 |
| 9 | 20.072 | 4.42026 | 327 | 23.4 | 0.447 | 9.1 | 0.21 |
| 10 | 21.035 | 4.22004 | 394 | 28.2 | 0.629 | 12.8 | 0.23 |
| 11 | 22.166 | 4.00714 | 1157 | 82.7 | 4.32 | 88.3 | 0.213 |
| 12 | 23.199 | 3.83094 | 341 | 24.4 | 0.842 | 17.2 | 0.279 |
| 13 | 27.329 | 3.26073 | 356 | 25.4 | 0.718 | 14.7 | 0.24 |

TABLE 10

| Equipment | Agilent HPLC1200 (LC -PDSC-02) |
|---|---|
| Mobile phase | A: Water containing 0.1% TFA; |
| | B: ACN containing 0.1% TFA; |
| | A: B (73:27) |
| Column | Symmetry C18, 75 mm × 4.6 mm, 3.5 um |
| | Lot No.: 0190382952 |
| UV Detector (nm) | 265 |
| Injection volume (μL) | 5 |
| Column temperature (° C.) | 25 |
| Flow rate (mL/min) | 1.0 |
| Run time (min) | 6 |
| t₀ (min) | 0.9 |
| t_{R} (min) | 3.7 |
| K′ | 3.1 |
| Tailing factor | 1.1 |

TABLE 11

| | | | Target | | | |
|---|---|---|---|---|---|---|
| | | | Conc. | | pH | HPLC |
| | Weight | Volume | (mg/ | Visual | (Fil- | Solubility |
| Media | (mg) | (mL) | mL) | Solubility | trated) | (mg/mL) |
| 0.1N HCl | 2.480 | 1.240 | 2.000 | >2 mg/mL | 1.00 | 2.073 |
| pH 2 | 2.834 | 1.416 | 2.000 | >2 mg/mL | 2.00 | 2.054 |
| pH 3 | 2.935 | 1.468 | 2.000 | >2 mg/mL | 2.96 | 2.028 |
| pH 4 | 3.033 | 1.516 | 2.000 | A few | 3.55 | 1.857 |
| | | | | particles | | |
| pH 5 | 2.631 | 1.316 | 2.000 | Turbid + | 3.63 | 1.106 |
| | | | | Many | | |
| | | | | particles | | |
| pH 6 | 2.464 | 1.232 | 2.000 | Many | 5.44 | 0.000 |
| | | | | particles | | |
| pH 7 | 2.867 | 1.434 | 2.000 | Many | 6.86 | 0.000 |
| | | | | particles | | |
| pH 8 | 2.929 | 1.464 | 2.000 | Many | 7.58 | 0.000 |
| | | | | particles | | |
| Water | 2.934 | 1.468 | 2.000 | >2 mg/mL | 3.45 | 2.036 |

TABLE 12

| | | | Target | | | |
|---|---|---|---|---|---|---|
| | | | Conc. | | pH | HPLC |
| | Weight | Volume | (mg/ | Visual | (Fil- | Solubility |
| Media | (mg) | (mL) | mL) | Solubility | trated) | (mg/mL) |
| 0.1N HCl | 2.547 | 1.274 | 2.000 | >2 mg/mL | 1.09 | 2.066 |
| pH 2 | 2.775 | 1.388 | 2.000 | >2 mg/mL | 2.17 | 2.056 |
| pH 3 | 2.141 | 1.070 | 2.000 | A few | 3.59 | 1.763 |
| | | | | particles | | |
| pH 4 | 2.866 | 1.432 | 2.000 | Turbid + | 4.72 | 0.011 |
| | | | | Many | | |
| | | | | particles | | |
| pH 5 | 3.035 | 1.518 | 2.000 | Turbid + | 5.27 | 0.001 |
| | | | | Many | | |
| | | | | particles | | |
| pH 6 | 2.642 | 1.320 | 2.000 | Turbid + | 6.04 | <0.001 |
| | | | | Many | | |
| | | | | particles | | |
| pH 7 | 2.621 | 1.310 | 2.000 | Turbid + | 7.00 | <0.001 |
| | | | | Many | | |
| | | | | particles | | |
| pH 8 | 2.875 | 1.438 | 2.000 | Turbid + | 7.91 | <0.001 |
| | | | | Many | | |
| | | | | particles | | |
| Water | 2.579 | 1.290 | 2.000 | Turbid + | 8.83 | <0.001 |
| | | | | Many | | |
| | | | | particles | | |

1. A solid form of a compound having Formula (I): 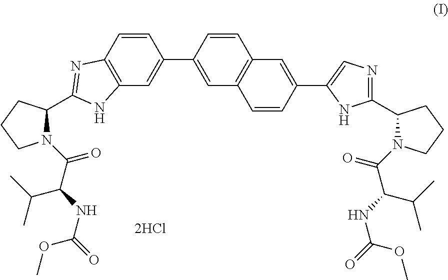 [SMILES: COC(=O)N[C@H](C(=O)N1CCC[C@H]1c1ncc(-c2ccc3cc(-c4ccc5nc([C@@H]6CCCN6C(=O)[C@@H](NC(=O)OC)C(C)C)[nH]c5c4)ccc3c2)[nH]1)C(C)C]
and in a crystalline form.

2. The solid form of claim 1 wherein the crystalline form is the Form A crystal form of the compound of Formula I.

3. The solid form of claim 1 wherein the solid form has an XRPD pattern comprising:
a) peaks located at 1, 2, 3, 4, 5, 6, 7, 8, 9, 10, 11, 12, 13, 14, 15, 16, 17, 18, 19, 20, 21, 22, 23, 24, 25, 26, 27, 28 or all of the approximate positions identified in Table 1;
b) peaks located at 1, 2, 3, 4, 5, 6, 7, 8, 9, 10, 11, 12, 13, 14, 15, 16, 17, 18, 19, 20, 21, 22, 23, 24, 25, 26 or all of the approximate positions identified in FIG. 6;
c) peaks located 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or all of the approximate positions identified in FIG. 8; or
d) peaks located at 1, 2, 3, 4, 5, 6, 7, 8, 9, 10, 11, 12, 13, 14, 15, 16, 17, 18 or all of the approximate positions identified in FIG. 19.

4. The solid form of claim 1 wherein the solid form has an XRPD pattern comprising peaks located at 1, 2, 3, 4 or all of the approximate positions identified in Table 2.

5. The solid form of claim 1 wherein the solid form has an XRPD pattern comprising peaks located at values of two theta of 14.7±0.2, 17.4±0.2, and one or more of 10.6±0.2, 12.7±0.2 and 13.6±0.1 at ambient temperature, based on a high quality pattern collected with a diffractometer (CuKα) with 2θ calibrated with an NIST or other suitable standard.

6. The solid form of claim 1 having a differential scanning calorimetry thermogram substantially like one of FIG. 4, 14, 21 or 23.

7. A pharmaceutical composition comprising the solid form of claim 1.

8. A gel capsule comprising the solid form of claim 1.

9. A solid form of a compound having Formula (II): 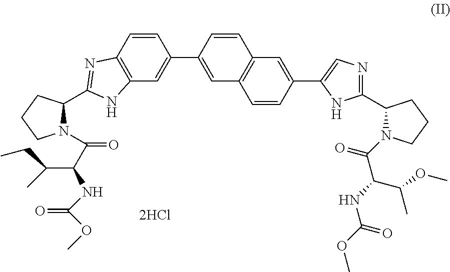 [SMILES: CC[C@H](C)[C@H](NC(=O)OC)C(=O)N1CCC[C@H]1c1nc2ccc(-c3ccc4cc(-c5cnc([C@@H]6CCCN6C(=O)[C@@H](NC(=O)OC)[C@@H](C)OC)[nH]5)ccc4c3)cc2[nH]1]
and having a crystalline form.

10. The solid form of claim 9 wherein the crystalline form is a Form I crystal form of the compound of Formula II.

11. The solid form of claim 9 wherein the solid form has an XRPD pattern comprising:
peaks located at 1, 2, 3, 4, 5, 6, 7, 8, 9, 10, 11, 12, 13, 14, 15, 16 or all of the approximate positions identified in Table 8 or
peaks located at 1, 2, 3, 4, 5, 6, 7, 8, 9, 10, 11, 12 or all of the approximate positions identified in Table 9.

12. The solid form of claim 9 wherein the solid form has an XRPD pattern comprising peak numbers 1, 3, 13 and 17 in Table 8 and 1, 2, 3, 4, 5, 6, 7, 8, 9, 10, 11, 12 or 13 of the remaining peaks identified in Table 8.

13. A pharmaceutical composition comprising the solid form of claim 9.
